(19) United States
(12) Patent Application Publication
(10) Pub. No.: US 2021/0196751 A1
(43) Pub. Date: Jul. 1, 2021

(54) METHODS OF CANCER TREATMENT USING AN ATR INHIBITOR

(71) Applicant: Vertex Pharmaceuticals Incorporated, Boston, MA (US)

(72) Inventors: Marina S. PENNEY, Acton, MA (US); John Robert POLLARD, Abingdon (GB); Darin TAKEMOTO, Belmont, MA (US); David GEHO, Sratham, NH (US); James SULLIVAN, Sudbury, MA (US); Philip Michael REAPER, Richmond, Surrey (GB)

(21) Appl. No.: 16/958,375

(22) PCT Filed: Dec. 27, 2018
(86) PCT No.: PCT/US2018/067673

§ 371 Date: Jun. 26, 2020

Related U.S. Application Data
(60) Provisional application No. 62/611,955, filed on Dec. 29, 2017.

(51) Int. Cl. A61K 33/243; A61K 31/282; A61K 31/513; A61K 31/7068; A61K 45/06; A61P 35/00

(52) U.S. Cl. CPC A61K 33/243; A61K 31/282; A61P 35/00; A61K 31/7068; A61K 45/06; A61K 31/513

(57) ABSTRACT

The present disclosure relates to methods of identifying a cancer having sensitivity to an ATR inhibitor compound, and treating subjects with such identified cancers with the ATR inhibitor, particularly in combination with a DNA damaging agent.

### CROSS-REFERENCE TO RELATED APPLICATIONS

This application claims the benefit of U.S. Provisional application No. 62/611,955, filed Dec. 29, 2017, the entire contents of which is incorporated herein by reference in its entirety.

### BACKGROUND

Cancer is considered a heterogeneous disease, where each cancer type is characterized by distinct macroscopic and molecular phenotype. This heterogeneity occurs between different cancer types and within a cancer and include differences in, among others, cellular morphology, microenvironment, gene expression, proliferation capacity, and metastatic potential. Genetic heterogeneity is a common characteristic, which can arise from the origin of the cancer itself, but also due to genetic instability from impaired DNA repair and cell replication machinery. In some instances, heterogeneity or selection of cancers with distinct features also arises from the selection pressure created by the cancer therapy itself. As a reflection of this heterogeneity, different cancers can exhibit different sensitivities to cancer treatments, and thus not all cancer patients respond equally to a prescribed cancer therapy and in fact, effectiveness of the cancer therapy shows high variability across different cancers. In addition, the sensitivity of a cancer to a particular therapy can vary with the stage of the cancer. Thus, it is desirable to have a basis for determining responsiveness of a cancer for selecting a particular cancer therapy, determining a dosing regimen, and assessing changes in responsiveness as the treatment and disease progresses.

### SUMMARY

It has been previously reported that certain ataxia telangiectasia mutated and Rad3 related (ATR) kinase inhibitors, referred to herein as ATR inhibitors or ATRi, synergize with certain chemotherapeutic agents. However, the ATR inhibitor combination therapy shows varying levels of synergy for different cancer types, and may have low synergy against some cancers. In the present disclosure, an analysis was conducted to identify biological markers having baseline expression levels that correlate with synergistic response to ATR inhibitors, particularly in combination with DNA damaging agents. This analysis examined about 18,000 markers and surprisingly identified cyclin-dependent kinase inhibitor-1 (CDKN1A) expression as a robust and statistically significant association for synergistic response. The analysis also identified an association of tumor protein 53 (TP53 or p53) mutational status with synergistic response to the ATR inhibitor combination therapy. However, the association of CDKN1A level was independent of TP53 status, indicating that CDKN1A possessed discriminatory power regardless of TP53 mutational status and/or function.

Moreover, the present disclosure describes the additional finding that CDKN1A levels below a particular threshold show a higher association to synergistic response. To this end, it is described herein that cancers having a CDKN1A level in the lower three quartiles (i.e., 1st to 3rd quartiles) as compared to the population tested displayed a higher association with a synergistic response to the ATR inhibitor combination therapy. In other words, cancers having the highest baseline CDKN1A levels were less likely to have a statistically significant association with a synergistic response to the ATR inhibitor combination therapy.

Accordingly, in one aspect, the present disclosure provides an ATR inhibitor for use in a method of treatment of cancer, characterized in the treatment being indicated in a cancer identified as having a reduced cyclin dependent kinase inhibitor 1A (CDKN1A) activity as compared to CDKN1A activity in a control tissue or cell. In some embodiments, the use is in a method of treating a cancer, comprising administering to a patient with a cancer identified as having a reduced CDKN1A activity as compared to CDKN1A activity in control tissue or cell a therapeutically effective amount of an ATR inhibitor to sensitize the cancer to a DNA damaging agent.

In some embodiments, identifying a cancer as having a reduced CDKN1A activity is by: measuring the level of CDKN1A activity in the cancer; and comparing the measured CDKN1A activity to CDKN1A activity in an appropriate control tissue or cell. As further discussed herein, in some embodiments, measuring of the CDKN1A activity is done in vitro, for example on a biological sample.

In some embodiments, the method of treatment further comprises detecting the presence or absence of an activity-attenuating or inactivating mutation in TP53 protein or a gene encoding the TP53 protein, wherein the cancer identified as having a reduced CDKN1A activity level compared to the CDKN1A activity in the control tissue or cell, and the presence of an activity-attenuating or inactivating mutation in the TP53 protein or the gene encoding the TP53 protein is administered a therapeutically effective amount of the ATR inhibitor.

In some embodiments, the method of treatment further comprises administering to the patient a therapeutically effective amount of one or more DNA damaging agents. In some embodiments, the therapeutically effective amount of a DNA damaging is that amount which is therapeutically effective in combination with the ATR inhibitor. In some embodiments, the therapeutically effective amount of the DNA damaging agent is less than the therapeutically effective amount of the DNA damaging agent when used in the absence of an ATR inhibitor.

In another aspect, the levels of CDKN1A activity is used to identify cancers having enhanced sensitivity to an ATR inhibitor. In some embodiments, a method of identifying a cancer having enhanced sensitivity to an ATR inhibitor comprises: measuring the level of CDKN1A activity in a cancer; comparing the measured CDKN1A activity to CDKN1A activity in an appropriate control tissue or cell; and identifying the cancer having a reduced CDKN1A activity compared to the CDKN1A activity in the control tissue or cell as having enhanced sensitivity to the ATR inhibitor.

In some embodiments, the enhanced sensitivity is to the ATR inhibitor in combination with a DNA damaging agent. In some embodiments, the enhanced sensitivity is characterized as a synergistic growth inhibition response of the cancer to the ATR inhibitor, particularly in combination with a DNA damaging agent.

In some embodiments, the method of identifying a cancer having enhanced sensitivity to an ATR inhibitor further comprises detecting the presence or absence of an activity-attenuating or inactivating mutation in TP53 protein or a gene encoding the TP53 protein, wherein the cancer having a reduced CDKN1A activity compared to the CDKN1A activity in the control tissue or cell and the presence of an activity-attenuating or inactivating mutation in the TP53 protein or the gene encoding the TP53 protein is identified as having an enhanced sensitivity to the ATR inhibitor.

In another aspect, the level of CDKN1A activity is used to select a cancer for treatment with the ATR inhibitor. In some embodiments, a method of selecting a cancer for treatment with an ATR inhibitor comprises: measuring the level of CDKN1A activity in a cancer; comparing the measured CDKN1A activity to CDKN1A activity in an appropriate control tissue or cell; and selecting the cancer having a reduced CDKN1A activity as compared to CDKN1A activity in the control tissue or cell for treatment with an ATR inhibitor.

In some embodiments, the method of selecting a cancer for treatment with the ATR inhibitor, further comprises detecting the presence or absence of an activity-attenuating or inactivating mutation in TP53 protein or a gene encoding the TP53 protein, wherein the cancer having a reduced CDKN1A activity compared to the CDKN1A activity in an appropriate control tissue or cell, and the presence of an activity-attenuating or inactivating mutation in the TP53 protein or the gene encoding the TP53 protein is selected for treatment with the ATR inhibitor.

In some embodiments, the selecting of the cancer is for treatment with the ATR inhibitor in combination with a DNA damaging agent.

In the embodiments of the present disclosure, a low or reduced CDKN1A activity is a CDKN1A activity level which is in the lower three quartiles of the CDKN1A activity in the control tissue or cell. In some embodiments, the reduced CDKN1A activity is a CDKN1A activity level which is in the third or lower quartile of the CDKN1A activity in the control tissue or cell. In some embodiments, the reduced CDKN1A activity is a CDKN1A activity level which is in the first quartile of the CDKN1A activity in the control tissue or cell. In some embodiments, a cut-off (or threshold) that demarcates those cancers less likely to respond synergistically than those cancers more likely to respond synergistically is between the bottom (lowest) three quartiles and the top (highest) single quartile of CDKN1A expression.

In some embodiments, a low or reduced CDKN1A activity is a CDKN1A activity level which is about 75% or less, about 50% or less, or about 25% or less of the CDKN1A activity of an appropriate control tissue or cell. In some embodiments, the reduced CDKN1A activity is a CDKN1A activity level which is about 50% or less of the CDKN1A activity of the control tissue or cell. In some embodiments, the reduced CDKN1A activity is a CDKN1A activity level which is about 25% or less of the CDKN1A activity of the control tissue or cell.

In a further aspect, the level of CDKN1A is used to identify a cancer or a patient with cancer contraindicated for treatment with the ATR inhibitor. In some embodiments, a method of identifying a patient having a cancer contraindicated or not indicated for treatment with an ATR inhibitor comprises: measuring the level of cyclin dependent kinase inhibitor 1A (CDKN1A) activity in a cancer of a patient; comparing the measured CDKN1A activity to CDKN1A activity in a control tissue or cell; and identifying the patient having a cancer with a CDKN1A activity which is substantially similar to CDKN1A activity in control tissue or cell as being contraindicated for treatment with the ATR inhibitor.

In some embodiments, the contraindication is for treatment of the cancer with an ATR inhibitor in combination with a DNA damaging agent.

In some embodiments, the cancer identified as being contraindicated for treatment with the ATR inhibitor has a measured CDKN1A activity in the fourth quartile of the CDKN1A activity in the control tissue or cell. In some embodiments, the cancer identified as being contraindicated for treatment with the ATR inhibitor has a measured CDKN1A activity which is greater than 75% of the CDKN1A activity of the control tissue or cell.

In some embodiments, the method of identifying a cancer as being contraindicated for treatment with the ATR inhibitor further comprises detecting the presence or absence of an activity-attenuating or inactivating mutation in TP53 protein or a gene encoding the TP53 protein, wherein the a cancer having a substantially similar CDKN1A activity compared to the CDKN1A activity in the control tissue or cell, and the absence of an activity-attenuating or inactivating mutation in the TP53 protein or the gene encoding the TP53 protein identifies the cancer as being contraindicated for treatment with the ATR inhibitor.

In some embodiments, the cancer for analysis, selection, and/or treatment according to the methods described herein include, but are not limited to, lung cancer (e.g., non-small cell lung cancer and small cell lung cancer), ovarian cancer, pancreatic cancer, head and neck cancer, glioma/glioblastoma, esophageal cancer, endometrial cancer, breast cancer, colorectal cancer, testicular cancer, liver cancer, prostate cancer and cervical cancer. Other cancers suitable for the methods herein are described in the detailed description.

In some embodiments, the ATR inhibitor for the methods and uses herein is a selective ATR inhibitor. In some embodiments, the ATR inhibitors include the compounds disclosed in published patent applications WO 2010/071837 and WO2014/089379. In some embodiments, the ATR inhibitor is a pyrazine compound encompassed by Formula IA, IIA, or IA-iii described herein, such as compounds disclosed in Table 1. In some embodiments, the ATR inhibitor is a pyrazolopyrimidine compound encompassed by formula I or IA, such as the compounds disclosed in Table 2 and Table 3. In some embodiments, the ATR inhibitor is a compound of formula:

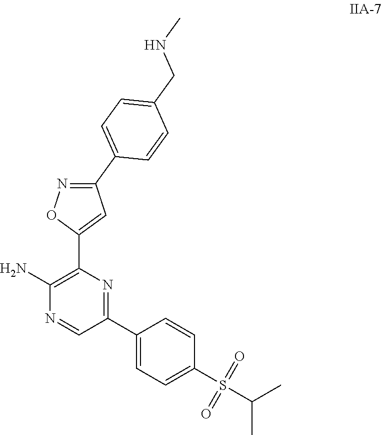 [SMILES: CNCc1ccc(-c2cc(-c3nc(-c4ccc(S(=O)(=O)C(C)C)cc4)cnc3N)on2)cc1]

or a pharmaceutically acceptable salt thereof.

In some embodiments, the methods described herein are for an ATR inhibitor, such as compound IIA-7, in combination with a DNA damaging agent. In some embodiments, the DNA-damaging agent is, by way of example and not limitation, ionizing radiation, platinating agent, topoisomerase I (Topo I) inhibitor, topoisomerase II (Topo II) inhibitor, anti-metabolite (e.g., purine antagonists and pyrimidine antagonists), alkylating agent, and anti-cancer antibiotic. In some embodiments, the DNA damaging agent is cisplatin or gemcitabine. In some embodiments, the combination therapy for the methods herein is ATR inhibitor compound IIA-7, or a pharmaceutically acceptable salt thereof, in combination with cisplatin. In some embodiments, the combination therapy for the methods herein is ATR inhibitor compound IIA-7, or a pharmaceutically acceptable salt thereof, in combination with gemcitabine.

In some embodiments, the methods described herein are for an ATR inhibitor, such as compound I-G-32, in combination with a DNA damaging agent. In some embodiments, the DNA-damaging agent is, by way of example and not limitation, ionizing radiation, platinating agent, topoisomerase I (Topo I) inhibitor, topoisomerase II (Topo II) inhibitor, anti-metabolite (e.g., purine antagonists and pyrimidine antagonists), alkylating agent, and anti-cancer antibiotic. In some embodiments, the DNA damaging agent is cisplatin or gemcitabine. In some embodiments, the combination therapy for the methods herein is ATR inhibitor compound I-G-32, or a pharmaceutically acceptable salt thereof, in combination with cisplatin. In some embodiments, the combination therapy for the methods herein is ATR inhibitor compound I-G-32, or a pharmaceutically acceptable salt thereof, in combination with gemcitabine.

In some embodiments, the ATR inhibitor is used in combination with an inhibitor of polyADP-ribose polymerase (PARP) or an inhibitor of Checkpoint-1 kinase (Chk1). Other second therapeutic agents for use with an ATR inhibitor in the methods of the present disclosure are provided herein. In some embodiments, the ATR inhibitor is used in combination with a DNA damaging agent, and a PARP inhibitor. In some embodiments, the ATR inhibitor is used in combination with a DNA damaging agent, and a Chk1 inhibitor. In some embodiments, the ATR inhibitor is used in combination with a DNA damaging agent, a PARP inhibitor, and a Chk1 inhibitor.

Various methods of measuring CDKN1A activity, assessing mutation status of CDKN1A protein and the gene encoding CDKN1A protein, and assessing mutation status of TP53 protein and the gene encoding TP53 protein are provided in the detailed description below. In some embodiments, the CDKN1A activity or level is measured by detecting CDKN1A protein, for example using an antibody against CDKN1A protein. In some embodiments, the CDKN1A activity or level is measured by detecting CDKN1A mRNA expression, for example by polymerase chain reaction or use of nucleic acid hybridization probes, such as on microarrays. In some embodiments, panels of probes or a probe set, such as a panel of nucleic acid probes, are used to measure expression of CDKN1A, and one or more of mutation status of CDKN1A and/or mutation status of TP53 in the cancer.

In another aspect, the present disclosure provides an article of manufacture comprising:

(a) a packaging material;

(b) an ATR inhibitor, or a pharmaceutically acceptable salt thereof; and

(c) a label, a package insert, or directions for obtaining the label or the package insert, contained within the packaging material, wherein the label or package insert provides prescribing information based on level of CDKN1A activity, or based on the level of CDKN1A activity and TP53 mutation status, determined for the cancer in the patient.

It should be appreciated that all combinations of the foregoing concepts and additional concepts discussed in greater detail below (provided such concepts are not mutually inconsistent) are contemplated as being part of the inventive subject matter disclosed herein.

### BRIEF DESCRIPTION OF THE FIGURES

It is to be understood that the Figures are not necessarily to scale, emphasis instead being placed upon generally illustrating the various concepts and embodiments discussed herein.

FIG. 1 shows plots of the synergy of ATR inhibitor compound I-G-32 or compound IIA-7 in combination with cisplatin or gemcitabine in a panel of 552 cancer cell lines. The bottom horizontal line (at a value of zero on the y-axis) represents no synergy (additive effect when agents are used in combination), the middle horizontal line represents synergy (equivalent to 3-fold IC50 shift (data not shown)), and the upper horizontal line represents strong synergy (equivalent to 10-fold IC50 shift (data not shown)). Cisplatin is denoted “cis,” and gemcitabine is denoted “gem.” The combination therapies are indicated in the x-axis.

FIG. 2 shows a boxplot of compound IIA-7 in combination with cisplatin by TP53 mutational status. The bottom, middle and upper horizontal lines are as described for FIG. 1. Wild-type denotes samples having no discernable TP53 mutation. Mutant denotes samples carrying a detected TP53 mutation.

FIG. 3 shows a boxplot of compound I-G-32 in combination with cisplatin by TP53 mutational status. The bottom, middle and upper horizontal lines are as described for FIG. 1. Wild-type denotes samples having no discernable TP53 mutation. Mutant denotes samples carrying a detected TP53 mutation.

FIG. 4 shows a boxplot of compound IIA-7 in combination with gemcitabine by TP53 mutational status. The bottom, middle and upper horizontal lines are as described for FIG. 1. Wild-type denotes samples having no discernable TP53 mutation. Mutant denotes samples carrying a detected TP53 mutation.

FIG. 5 shows a boxplot of compound I-G-32 in combination with gemcitabine by TP53 mutational status. The bottom, middle and upper horizontal lines are as described for FIG. 1. Wild-type denotes samples having no discernable TP53 mutation. Mutant denotes samples carrying a detected TP53 mutation.

FIG. 6 shows a scatterplot of baseline CDKN1A gene expression versus compound IIA-7/cisplatin synergy colored by TP53 mutational status. Each dot represents a different cancer cell line.

FIG. 7 shows a scatterplot of baseline CDKN1A gene expression versus compound I-G-32/cisplatin synergy colored by TP53 mutational status. Each dot represents a different cancer cell line.

FIG. 8 shows a scatterplot of baseline CDKN1A gene expression versus compound IIA-7/gemcitabine synergy colored by TP53 mutational status. Each dot represents a different cancer cell line.

FIG. 9 shows a scatterplot of baseline CDKN1A gene expression versus compound I-G-32/gemcitabine synergy colored by TP53 mutational status. Each dot represents a different cancer cell line.

FIG. 10 shows a boxplot of compound IIA-7 in combination with cisplatin by CDKN1A gene expression quartiles. The bottom, middle and upper horizontal lines are as described for FIG. 1. 4Q denotes the samples having the highest (top 25%) CDKN1A expression, when the CDKN1A expression is divided into 4 equal parts in a log scale. 1-3Q denotes the samples having the lowest (bottom 75%) CDKN1A expression. The number of samples in each group (either Q4 or 1-3Q) are shown in parentheses.

FIG. 11 shows a boxplot of compound I-G-32 in combination with cisplatin by CDKN1A gene expression quartiles. The bottom, middle and upper horizontal lines are as described for FIG. 1. The 4Q and 1-3Q groups are as defined for FIG. 10.

FIG. 12 shows a boxplot of compound IIA-7 in combination with gemcitabine by CDKN1A gene expression quartiles. The bottom, middle and upper horizontal lines are as described for FIG. 1. The 4Q and 1-3Q groups are as defined for FIG. 10.

FIG. 13 shows a boxplot of compound I-G-32 in combination with gemcitabine by CDKN1A gene expression quartiles. The bottom, middle and upper horizontal lines are as described for FIG. 1. The 4Q and 1-3Q groups are as defined for FIG. 10.

FIG. 14 shows analysis of variance (ANOVA) results for the association between TP53 mutation status and baseline CDKN1A gene expression and activity of compound IIA-7 or compound I-G-32 in combination with cisplatin or gemcitabine.

### DETAILED DESCRIPTION

The present disclosure provides a method of identifying a cancer sensitive to cancer therapy with an ATR inhibitor, and its use as a basis for selecting a cancer for treatment with the cancer therapy. Biomarkers for identifying a cancer sensitive to the cancer therapy were identified by screening over 500 cancer cell lines, encompassing a range of cancer types, for baseline expression of about 18,000 expressed genes. The cell lines were further assessed for their response to a combination therapy comprising an ATR inhibitor and a DNA damaging agent, particularly the combination of the ATR inhibitor with a platinating agent, such as cisplatin, or an anti-metabolite, such as gemcitabine. The cytotoxic effects of these combinations on these cell lines ranged from less than additive, to additive to synergistic.

Of about 18,000 expressed genes tested, the expression of cyclin dependent kinase inhibitor 1A (CDKN1A) appeared to robustly track with and thus associate with degree of sensitivity of the cancer to the combination of the ATR inhibitor and the DNA damaging agent. The screening also identified TP53 protein to also associate with sensitivity of the cancer to the combination treatment. While TP53 has some association to overall response to ATR inhibitor combination therapies, the experimental data provided herein indicate that CDKN1A has stronger association with sensitivity to the cancer therapy than TP53. That this single gene product out of 18,000 had a strong association with the degree of sensitivity to the combination therapeutic is surprising. Even more unexpected is that the association of sensitivity with CDKN1A expression was independent of TP53 level or mutational status. The identification of CDKN1A expression for discriminating between cancers showing synergistic response and cancers with low or non-synergistic response is valuable as it allows the treatments with the ATR inhibitor to be used in those patients most likely to benefit and spare those who are not likely to benefit. Accordingly, the present disclosure provides methods for identifying, selection and treatment of cancers with an ATR inhibitor based on CDKN1A activity, and in some embodiments, based on CDKN1A activity and TP53 mutational status.

As used in this specification and the appended claims, the singular forms “a”, “an” and “the” include plural referents unless the context clearly indicates otherwise. Thus, for example, reference to “a protein” includes more than one protein, and reference to “a compound” refers to more than one compound.

Also, the use of “or” means “and/or” unless stated otherwise. Similarly, “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

It is to be understood that both the foregoing general description, including the drawings, and the following detailed description are exemplary and explanatory only and are not restrictive of this disclosure. The section headings used herein are for organizational purposes only and not to be construed as limiting the subject matter described.

Selection and Treatment of Cancers

In the present disclosure and as understood in the art, cyclin dependent kinase inhibitor 1A, (CDKN1A) is characterized as a protein that binds to and inhibits the activity of cyclin-dependent kinases, such as cyclin-CDK2, -CDK1, and -CDK4/6 complexes. It acts as a regulator of cell cycle progression at G1, and its expression is believed to be tightly controlled by TP53 (see, e.g., Ulu et al., 2003, J Biol Chem 278:32507-32516). It has been suggested that TP53-dependent cell cycle arrest at G1 in response to various stress stimuli is mediated through CDKN1A (see, e.g., Abbas et al., 2009, Nat Rev Cancer. 9(6):400-414). In addition to its designation as cyclin dependent kinase inhibitor 1A, CDKN1A is also known in the art by a number of other names including cyclin-dependent kinase inhibitor-1, CDK-interacting (or interaction) protein 1, CIP1, p21, p21CIP, WAF1, wildtype p53-activated fragment 1, p21Waf1, CAP20, MDA-6, melanoma differentiation associated protein 6, SDI1, and PIC1. These terms may be used interchangeably herein.

An exemplary human CDKN1A protein is 164 amino acids in length, and an exemplary amino acid sequence is available at the NCBI database as accession number CAG38770. The amino acid sequence is as follows:

(SEQ ID NO: 1)
MSEPAGDVRQ NPCGSKACRR LFGPVDSEQL SRDCDALMAG
CIQEARERWN FDFVTETPLE GDFAWERVRG LGLPKLYLPT
GPRRGRDELG GGRRPGTSPA LLQGTAEEDH VDLSLSCTLV
PRSGEQAEGS PGGPGDSQGR KRRQTSMTDF YHSKRRLIFS
KRKP.

The CDKN1A mRNA (cDNA clone RZPDo834A0522D) encoding the foregoing protein is about 495 base pairs, and its sequence is available in the NCBI database as accession number CR536533. The nucleotide sequence of the cDNA is as follows:

(SEQ ID NO: 2)
ATGTCAGAAC CGGCTGGGGA TGTCCGTCAG AACCCATGCG
GCAGCAAGGC CTGCCGCCGC CTCTTCGGCC CAGTGGACAG
CGAGCAGCTG AGCCGCGACT GTGATGCGCT AATGGCGGGC
TGCATCCAGG AGGCCCGTGA GCGATGGAAC TTCGACTTTG
TCACCGAGAC ACCACTGGAG GGTGACTTCG CCTGGGAGCG
TGTGCGGGGC CTTGGCCTGC CCAAGCTCTA CCTTCCCACG
GGGCCCCGGC GAGGCCGGGA TGAGTTGGGA GGAGGCAGGC
GGCCTGGCAC CTCACCTGCT CTGCTGCAGG GGACAGCAGA
GGAAGACCAT GTGGACCTGT CACTGTCTTG TACCCTTGTG
CCTCGCTCAG GGGAGCAGGC TGAAGGGTCC CCAGGTGGAC
CTGGAGACTC TCAGGGTCGA AAACGGCGGC AGACCAGCAT
GACAGATTTC TACCACTCCA AACGCCGGCT GATCTTCTCC
AAGAGGAAGC CCTAA.

As used herein, CDKN1A encompasses variants, including orthologs and interspecies mammalian homologs, of the human CDKN1A. In some embodiments, while the exemplary description herein on use of CDKN1A expression for identifying a cancer sensitive to an ATR inhibitor are described with respect to human patients, it is to be understood that it can also be applied to appropriate mammalian species. As used herein, “identified” or “identifying” refers to analyzing for, detection of, or carrying out a process for the presence or absence of one or more specified characteristics.

Thus, in one aspect, the present disclosure provides an ATR inhibitor for use in a method of treatment of cancer, characterized in the treatment being indicated in a cancer identified as having a reduced cyclin dependent kinase inhibitor 1A (CDKN1A) activity as compared to CDKN1A activity in a control tissue or cell.

In some embodiments, this disclosure provides the above ATR inhibitor further characterized in the treatment being in combination with a DNA damaging agent.

In some embodiments, this disclosure provides a method of treating a patient having cancer, comprising administering to a patient with a cancer identified as having a reduced cyclin dependent kinase inhibitor 1A (CDKN1A) activity as compared to CDKN1A activity in control tissue or cell a therapeutically effective amount of an ATR inhibitor to sensitize the cancer to a DNA damaging agent.

In some embodiments, identifying a cancer as having a reduced CDKN1A activity is by: (a) measuring the level of CDKN1A activity in the cancer; and (b) comparing the measured CDKN1A activity to CDKN1A activity in a control tissue or cell. As further discussed herein, in some embodiments, measuring of the CDKN1A activity is done in vitro, for example on a biological sample.

In some embodiments, the method of treatment further comprises administering to the patient a therapeutically effective amount of a DNA damaging agent.

In some embodiments, a method of treating a patient having cancer comprises administering to a patient having a cancer identified as having a reduced CDKN1A activity as compared to CDKN1A activity in control tissue or cell a therapeutically effective amount of an ATR inhibitor in combination with a DNA damaging agent. As noted above, in some embodiments, the therapeutically effective amount of a DNA damaging is that amount which is therapeutically effective in combination with the ATR inhibitor. In some embodiments, the therapeutically effective amount of the DNA damaging agent is less than the therapeutically effective amount of the DNA damaging agent when used in the absence of an ATR inhibitor.

In some embodiments, a method of treating a patient having cancer comprises: measuring the level of CDKN1A activity in a cancer of a patient afflicted with the cancer; comparing the measured CDKN1A activity to CDKN1A activity in a control tissue or cell; and administering to the patient with the cancer identified as having a reduced CDKN1A activity as compared to CDKN1A activity in control tissue or cell a therapeutically effective amount of an ATR inhibitor to sensitize the cancer to a DNA damaging agent.

In some embodiments, the method of treating a subject with cancer based on measuring the level of CDKN1A activity in the cancer further comprises administering to the patient a therapeutically effective amount of a DNA damaging agent.

Thus, in some embodiments, a method of treating a patient having cancer comprises: measuring the level of cyclin dependent kinase inhibitor 1A (CDKN1A) activity in a cancer of a patient afflicted with the cancer; comparing the measured CDKN1A activity to CDKN1A activity in a control tissue or cell; and administering to the patient with the cancer identified as having a reduced CDKN1A activity as compared to CDKN1A activity in control tissue or cell a therapeutically effective amount of an ATR inhibitor in combination with a DNA damaging agent.

As further discussed herein, the ATR inhibitor and the DNA damaging agent can be administered sequentially or concurrently, together or separately, by the same route or by different route, as appropriate for the combination treatment. In some embodiments, the ATR inhibitor is administered followed by administration of the DNA damaging agent. In some embodiments, the DNA damaging agent is administered followed by administration of the ATR inhibitor. In some embodiments, wherein the ATR inhibitor and the DNA damaging agent are administered sequentially, sufficient time is provided between their administration to enhanced the effectiveness of the combination therapy, as further described herein.

In some embodiments of the treatment, the cancer having a reduced CDKN1A activity is characterized by a synergistic growth inhibition response to the ATR inhibitor and the DNA damaging agent. In some embodiments, the treatment regimen with the ATR inhibitor and the DNA damaging agent are made to provide high synergistic anti-cancer activity, e.g., high synergistic inhibition of cancer cell growth.

In some embodiments, the method of treatment further comprises detecting the presence or absence of an activity-attenuating or inactivating mutation in TP53 protein or a gene encoding the TP53 protein, wherein the cancer identified as having a reduced CDKN1A activity level compared to the CDKN1A activity in the control tissue or cell, and the presence of an activity-attenuating or inactivating mutation in the TP53 protein or the gene encoding the TP53 protein is administered a therapeutically effective amount of the ATR inhibitor.

In some embodiments, the activity attenuating or inactivating mutation of TP53 in the cancer for treatment with the ATR inhibitor is a loss of function mutation in the DNA binding domain, homo-oligomerization domain, or transactivation domain of TP53.

In another aspect, the level of CDKN1A activity is used to identify a cancer having enhanced sensitivity to an ATR inhibitor. In some embodiments, a method of identifying a cancer having enhanced sensitivity to an ATR inhibitor comprises: measuring the level of CDKN1A activity in a cancer; comparing the measured CDKN1A activity to CDKN1A activity in a control tissue or cell; and identifying the cancer having a reduced CDKN1A activity compared to the CDKN1A activity in the control tissue or cell as having enhanced sensitivity to the ATR inhibitor.

In some embodiments of identifying a cancer having enhanced sensitivity to an ATR inhibitor, the enhanced sensitivity is to the ATR inhibitor in combination with a DNA damaging agent. In some embodiments, the enhanced sensitivity is a synergistic growth inhibition response to the ATR inhibitor in combination with the DNA damaging agent.

In some embodiments, the method of identifying a cancer having enhanced sensitivity to an ATR inhibitor further comprises detecting the presence or absence of an activity-attenuating or inactivating mutation in TP53 protein or a gene encoding the TP53 protein, wherein the cancer having a reduced CDKN1A activity compared to the CDKN1A activity in the control tissue or cell, and the presence of an activity-attenuating or inactivating mutation in the TP53 protein or the gene encoding the TP53 protein is identified as having an enhanced sensitivity to the ATR inhibitor.

In some embodiments, the activity attenuating or inactivating mutation of TP53 for identifying a cancer having an enhanced sensitivity to the ATR inhibitor is a loss of function mutation in the DNA binding domain, homo-oligomerization domain, or transactivation domain of TP53.

In another aspect, the level of CDKN1A activity is used to select a cancer for treatment with the ATR inhibitor. In some embodiments, a method of selecting a cancer for treatment with an ATR inhibitor comprises: measuring the level of cyclin dependent kinase inhibitor 1A (CDKN1A) activity in a cancer; comparing the measured CDKN1A activity to CDKN1A activity in a control tissue or cell; and selecting the cancer having a reduced CDKN1A activity as compared to CDKN1A activity in a control tissue or cell for treatment with an ATR inhibitor.

In some embodiments, the selecting of the cancer is for treatment with the ATR inhibitor in combination with a DNA damaging agent.

In some embodiments, the cancer having a reduced CDKN1A activity and selected for treatment is characterized by a synergistic growth inhibition response to the ATR inhibitor and a DNA damaging agent.

In some embodiments, the method of selecting a cancer for treatment with the ATR inhibitor, further comprises detecting the presence or absence of an activity-attenuating or inactivating mutation in TP53 protein or a gene encoding the TP53 protein, wherein the cancer having a reduced CDKN1A activity compared to the CDKN1A activity in the control tissue or cell, and the presence of an activity-attenuating or inactivating mutation in the TP53 protein or the gene encoding the TP53 protein is selected for treatment with the ATR inhibitor.

In some embodiments, the activity attenuating or inactivating mutation of TP53 for selecting the cancer for treatment with the ATR inhibitor is a loss of function mutation in the DNA binding domain, homo-oligomerization domain, or transactivation domain of TP53.

In another aspect, the level of CDKN1A activity is used to identify a patient with a cancer having an enhanced sensitivity to an ATR inhibitor. In some embodiments, a method of identifying a patient with a cancer having enhanced sensitivity to treatment with an ATR inhibitor comprises: measuring the level of cyclin dependent kinase inhibitor 1A (CDKN1A) activity in a cancer of a patient; comparing the measured CDKN1A activity to CDKN1A activity in a control tissue or cell; and identifying the patient with a cancer having a reduced CDKN1A activity as compared to CDKN1A activity in control tissue or cell as having an enhanced sensitivity to treatment with the ATR inhibitor.

In some embodiments of identifying a patient with a cancer having enhanced sensitivity to an ATR inhibitor, the enhanced sensitivity is to the ATR inhibitor in combination with a DNA damaging agent.

In some embodiments, the enhanced sensitivity is a synergistic growth inhibition response to the ATR inhibitor in combination with a DNA damaging agent.

In another aspect, the level of CDKN1A is used to select a patient with cancer for treatment with the ATR inhibitor. In some embodiments, a method of selecting a patient with cancer for treatment with an ATR inhibitor comprises: measuring the level of CDKN1A activity in a cancer of a patient; comparing the measured CDKN1A activity to CDKN1A activity in a control tissue or cell; and selecting the patient with a cancer identified as having a reduced CDKN1A activity as compared to CDKN1A activity of a control tissue or cell for treatment with the ATR inhibitor.

In some embodiments, the selection of a patient with cancer is for treatment with the ATR inhibitor in combination with a DNA damaging agent.

In some embodiments in the selection of the patient, the cancer identified as having a reduced CDKN1A activity is characterized by a synergistic growth inhibition response to the ATR inhibitor and a DNA damaging agent.

In some embodiments, the method of selecting a patient for treatment with the ATR inhibitor further comprises detecting the presence or absence of an activity-attenuating or inactivating mutation in TP53 protein or a gene encoding the TP53 protein, wherein the patient with the cancer having a reduced CDKN1A activity compared to the CDKN1A activity in the control tissue or cell, and the presence of an activity-attenuating or inactivating mutation in the TP53 protein or the gene encoding the TP53 protein is selected for treatment with the ATR inhibitor.

In some embodiments of the method of selecting a patient for treatment with the ATR inhibitor, the activity attenuating or inactivating mutation of TP53 is a loss of function mutation in the DNA binding domain, homo-oligomerization domain, or transactivation domain of TP53.

As provided herein, a low or reduced level of CDKN1A activity, e.g., mRNA expression, is associated with responsiveness of the cancer to the ATR inhibitor, particularly in combination with a DNA damaging agent. In some embodiments, for any of the methods and uses described herein, for example without limitation, treatment of a cancer, identifying a cancer, or selecting a cancer for treatment with the ATR inhibitor, the reduced CDKN1A activity is a CDKN1A activity level which is in the lower three quartiles of the CDKN1A activity in the control tissue or cell. In some embodiments, the reduced CDKN1A activity is a CDKN1A activity level which is in the third or lower quartile of the CDKN1A activity in the control tissue or cell. In some embodiments, the reduced CDKN1A activity is a CDKN1A activity level which is in the first quartile of the CDKN1A activity in the control tissue or cell. In some embodiments, a cut-off (or threshold) that demarcates those patients less likely to respond synergistically than those more likely to respond synergistically is between the bottom (lowest) three quartiles and the top (highest) single quartile of CDKN1A expression.

In some embodiments, for any of the methods and uses described herein, for example without limitation, treatment of a cancer, identifying a cancer, or selecting a cancer for treatment with the ATR inhibitor, the reduced CDKN1A activity is a CDKN1A activity level which is about 75% or less, about 50% or less, or about 25% or less of the CDKN1A activity of the control tissue or cell. In some embodiments, the reduced CDKN1A activity is a CDKN1A activity level which is about 50% or less of the CDKN1A activity of the control tissue or cell. In some embodiments, the reduced CDKN1A activity is a CDKN1A activity level which is about 25% or less of the CDKN1A activity of the control tissue or cell.

In a further aspect, the level of CDKN1A is used to identify a cancer or a patient with cancer contraindicated for treatment with the ATR inhibitor. In some embodiments, a method of identifying a patient having a cancer contraindicated or not indicated for treatment with an ATR inhibitor, comprises: measuring the level of cyclin dependent kinase inhibitor 1A (CDKN1A) activity in a cancer of a patient; comparing the measured CDKN1A activity to CDKN1A activity in a control tissue or cell; and identifying the patient having a cancer with a CDKN1A activity which is substantially similar to CDKN1A activity in control tissue or cell as being contraindicated for treatment with the ATR inhibitor.

In some embodiments of identifying a cancer or a patient with cancer contraindicated for treatment with the ATR inhibitor, the contraindication is for treatment with the ATR inhibitor in combination with a DNA damaging agent.

In some embodiments, the patient having a cancer identified as being contraindicated for treatment with the ATR inhibitor is not selected for treatment with the ATR inhibitor, or not selected for treatment with the ATR inhibitor in combination with a DNA damaging agent. In some embodiments, the patient identified as having a cancer as being contraindicated for treatment with the ATR inhibitor is treated with cancer therapy other than treatment with the ATR inhibitor or other than treatment with the ATR inhibitor in combination with a DNA damaging agent.

In some embodiments, the cancer contraindicated for treatment with the ATR inhibitor is characterized by a by a non-synergistic growth inhibition response to the ATR inhibitor in combination with a DNA damaging agent. In some embodiments, the cancer having substantially similar CDKN1A activity as compared to control tissue or cell is characterized by a non-synergistic growth inhibition response to the ATR inhibitor and a DNA damaging agent.

In some embodiments, the cancer identified as being contraindicated for treatment with the ATR inhibitor has a measured CDKN1A activity in the fourth quartile of the CDKN1A activity in the control tissue or cell. In some embodiments, the cancer identified as being contraindicated for treatment with the ATR inhibitor has a measured CDKN1A activity which is greater than 75% of the CDKN1A activity of the control tissue or cell. In some embodiments, a substantially similar level of CDKN1A activity to level of CDKN1A activity in control tissue or cell is CDKN1A activity in the fourth quartile of the CDKN1A activity in the control tissue or cell, or in some embodiments, greater than 75% of the CDKN1A activity of the control tissue or cell. In some embodiments, a substantially similar activity to level of CDKN1A activity in control tissue or cell is CDKN1A activity which is greater than 80%, greater than 85%, greater than 90%, or greater than 95% or more of the CDKN1A activity in control tissue or cell.

In some embodiments, the method of identifying a cancer or a patient with cancer as being contraindicated for treatment with the ATR inhibitor further comprises detecting the presence or absence of an activity-attenuating or inactivating mutation in TP53 protein or a gene encoding the TP53 protein, wherein the patient with a cancer having a substantially similar CDKN1A activity compared to the CDKN1A activity in the control tissue or cell, and the absence of an activity-attenuating or inactivating mutation in the TP53 protein or the gene encoding the TP53 protein identifies the patient as being contraindicated for treatment with the ATR inhibitor. In some embodiments, the activity attenuating or inactivating mutation of TP53 is a loss of function mutation in the DNA binding domain, homo-oligomerization domain, or transactivation domain of TP53. In some embodiments, the cancer contraindicated for treatment with the ATR inhibitor expresses a wild-type TP53 protein.

In another aspect, the level of CDKN1A activity is used to select a cancer treatment regimen for a patient diagnosed with cancer. In some embodiments, a method of selecting a cancer treatment regimen for a patient having a cancer comprises: measuring the level of cyclin dependent kinase inhibitor 1A (CDKN1A) activity in a cancer of a patient; comparing the measured CDKN1A activity to CDKN1A activity in a control tissue or cell; and (a) selecting a cancer treatment regimen that does not include treatment with an ATR inhibitor in combination with a DNA damaging agent if the cancer is identified as having a CDKN1A activity which is substantially similar to CDKN1A activity in control tissue or cell; and (b) selecting a cancer treatment regimen that includes treatment with an ATR inhibitor in combination with a DNA damaging agent if the cancer is identified as having a CDKN1A activity which is reduced as compared to CDKN1A activity of control tissue or cell.

In a further aspect, a method of treating a patient having a cancer comprises: measuring the level of cyclin dependent kinase inhibitor 1A (CDKN1A) activity in a cancer of a patient; comparing the measured CDKN1A activity to CDKN1A activity in a control tissue or cell; and (a) treating the patient with a cancer treatment regimen which does not include treatment with an ATR inhibitor in combination with a DNA damaging agent if the cancer is identified as having a CDKN1A activity which is substantially similar to CDKN1A activity in control tissue or cell; and (b) treating the patient with a cancer treatment regimen which includes treatment with an ATR inhibitor in combination with a DNA damaging agent if the cancer is identified as having a CDKN1A activity which is reduced as compared to CDKN1A activity in control tissue or cell.

In another aspect, the present disclosure provides an article of manufacture comprising:

(a) a packaging material;

(b) an ATR inhibitor, or a pharmaceutically acceptable salt thereof; and

(c) a label, a package insert, or directions for obtaining the label or the package insert, contained within the packaging material, wherein the label or package insert provides prescribing information based on level of CDKN1A activity, or based on the level of CDKN1A activity and TP53 mutations status, determined for the cancer in the patient.

In some embodiments, the label or the package insert provides one or more of the following prescribing information: (i) treatment with the ATR inhibitor in combination with a DNA damaging agent is recommended for patients with a cancer having a reduced CDKN1A expression compared to appropriate controls; (ii) treatment with the ATR inhibitor in combination with a DNA damaging agent is recommended for patients with a cancer having CDKN1A expression which is about 75% or less, or about 50% or less, or about 25% or less of appropriate controls; (iii) treatment with the ATR inhibitor in combination with a DNA damaging agent is recommended for patients with a cancer having CDKN1A expression which is in the third or lower quartile of appropriate controls; (iv) select patients with cancer having a reduced CDKN1A expression compared to appropriate controls for therapy with the ATR inhibitor in combination with a DNA damaging agent; (v) select patients with cancer having CDKN1A expression which is about 75% or less, or about 50% or less, or about 25% or less of appropriate controls for therapy with the ATR inhibitor in combination with a DNA damaging agent; (vi) select patients with cancer having CDKN1A expression which is in the third or lower quartile of appropriate controls for therapy with the ATR inhibitor in combination with a DNA damaging agent; (vii) treatment with the ATR inhibitor in combination with a DNA damaging agent is not indicated or is contraindicated for patients with a cancer having CDKN1A expression which is not reduced or is substantially similar compared to .CDKN1A expression in appropriate controls; (viii) treatment with the ATR inhibitor in combination with a DNA damaging agent is not indicated or is contraindicated for patients with a cancer having CDKN1A expression which is in the fourth quartile of appropriate controls; and (ix) treatment with the ATR inhibitor in combination with a DNA damaging agent is not indicated or is contraindicated for patients with a cancer having CDKN1A expression which is greater than 75% of appropriate controls.

In the various embodiments herein, “control tissue,” “control cell,” “control sample,” “reference tissue,” “reference cell,” or “reference sample,” as used herein refers to a sample, cell, tissue, standard, or level that is used for comparison purposes. For example, the level of CDKN1A activity of a cancer is compared to the level of CDKN1A activity in a control tissue, control cell, control sample, reference tissue, reference cell, or reference sample for treatment of a cancer, identifying a cancer, or selecting a cancer for treatment with the ATR inhibitor. In some embodiments, the control tissue, control cell, control sample, reference tissue, reference cell, or reference sample is obtained from a healthy and/or non-diseased part of the body (e.g., tissue or cells) of the same subject or individual or a group of such individuals. In some embodiments, the control tissue, control cell, control sample, reference tissue, reference cell, or reference sample is obtained from a healthy and/or non-diseased part of the body (e.g., tissues or cells) of an individual who is not the subject or patient or a group of such individuals. In some embodiments, the control tissue, control cell, control sample, reference tissue, reference cell, or reference sample is a non-cancerous tissue or non-cancerous cell. In some embodiments, the control tissue, control cell, control sample, reference tissue, reference cell, or reference sample is normal tissue or normal cell. In some embodiments, the normal tissue or normal cell is a tissue type or cell type determined for the cancer.

In some embodiments, the control tissue, control cell, control sample, reference tissue, reference cell, or reference sample is control tissue or cell which is characterized by a non-synergistic growth inhibition response to the ATR inhibitor, particularly in the response to the ATR inhibitor in combination with a DNA damaging agent. In some embodiments, the control tissue, control cell, control sample, reference tissue, reference cell, or reference sample is control cancer tissue or cancer cell characterized by a non-synergistic growth inhibition response to the ATR inhibitor in combination, particularly in the response to the ATR inhibitor in combination with a DNA damaging agent. In some embodiments, the control cancer tissue or cancer cell are of the tissue type or cell type determined for the cancer being evaluated for levels of CDKN1A activity.

In some embodiments, the CDKN1A activity is determined by: (a) measuring CDKN1A protein expression, (b) measuring CDKN1A mRNA expression, (c) detecting the presence or absence of activity-attenuating or inactivating mutations in CDKN1A protein or a gene encoding the CDKN1A protein, or (d) combinations of the foregoing. In some embodiments, the measuring of CDKN1A activity is done in vitro, for example on biological samples obtained from the subject, including among others, cells or tissues.

In some embodiments, the CDKN1A activity is determined by measuring CDKN1A protein expression. In some embodiments, measuring the CDKN1A protein expression is with a binding agent which specifically binds to CDKN1A protein. In some embodiments, measuring the CDKN1A protein expression is with an antibody which specifically binds to CDKN1A protein. In some embodiments, the antibody used binds to one or more variants of CDKN1A protein, such as splicing variants or polymorphic variants. Various antibody-based protein detection techniques for the uses herein include, by way of example and not limitation, enzyme linked immunosorbent assay (ELISA), immunohistochemistry, immunocytochemistry, fluorescence polarization immunoassay, and Western blotting. In some embodiments, measuring the CDKN1A protein expression is by fluorescence activated cell sorting (FACS) of cells, for example, using permeabilized cancer cells or control cells (see, e.g., Watanabe et al., 2010, J Virol. 84(14):6966-6977). In some embodiments, the binding agent can be an aptamer (e.g., peptide or nucleic acid) which specifically binds to the CDKN1A or TP53 protein (see, e.g., US20130059292; Chen et al., 2015, Proc Natl Acad Sci USA. 112(32):10002-10007, incorporated herein by reference). In some embodiments, the CDKN1A protein can be detected using a microarray of binding agents. In some embodiments, measuring the CDKN1A protein levels uses a panel of antibodies directed against human CDKN1A. In some embodiments, at least one of the antibodies is capable of binding to all variants of human CDKN1A protein, including human CDKN1A protein, isoform 1 and isoform 2. In some embodiments, the panel of antibodies includes one or more antibodies capable of binding to a control expression product, for example, actin, glyceraldehyde 3-phosphate dehydrogenase, α-tubulin, Mapk1, and/or β2-microglobulin, preferably its human forms.

In some embodiments, panel of probes, e.g., antibodies, directed against human CDKN1A also includes probes capable of detecting expression level or mutational status of TP53. In some embodiments, the panel of probes for detecting CDKN1A activity includes one or more antibodies which specifically bind to CDKN1A protein, and one or more antibodies capable of detecting TP53 protein levels. In some embodiments, the panel of probes further includes one or more antibodies capable of detecting activity attenuating or inactivating mutations in TP53 protein.

In some embodiments, the CDKN1A activity is determined by measuring CDKN1A mRNA expression. In some embodiments, the level of CDKN1A mRNA expression is determined by hybridization to a nucleic acid probe, such as hybridization to a nucleic acid with a sequence complementary to the CDKN1A mRNA sequence or other CDKN1A expressed sequences. In some embodiments, the CDKN1A mRNA expression can be determined by Northern hybridization. In some embodiments, the CDKN1A mRNA expression is determined by hybridization to a nucleic acid microarray. In some embodiments, the CDKN1A mRNA expression is measured by polymerase chain reaction (PCR), including RT-PCR (i.e., reverse transcription polymerase chain reaction). In some embodiments, the PCR is quantitative PCR, for example Real Time qRT-PCR (i.e., Real Time Quantitative Reverse Transcription PCR). In some embodiments, for PCR analysis, for example Real Time PCR, such as TaqMan®, the primer probes are directed to the exons of the human CDKN1A gene sequence, for example, the boundary of exons 1-2, 2-3, 3-4, 4-5, and/or 5-6. In some embodiments where the CDKN1A mRNA expression is measured by hybridization to nucleic acid probes, for example in a microarray, the biological sample obtained from the subject is contacted with a panel of nucleic acid probes, where at least one probe hybridizes to a exon common to all splice variants of the expressed CDKN1A mRNA, particularly its human form. In some embodiments, the panel of nucleic acid probes includes one or more nucleic acids which hybridize to unique sequences of splice variants, particularly splice variants of human CDKN1A mRNA, for example, CDKN1A splice variant 1, CDKN1A variant 2, CDKN1A variant 3, CDKN1A variant 4, and/or CDKN1A variant 5 (see, e.g., Nozell et al., 2002, Oncogene 21, 1285-1294; Kreis et al., 2008, J Neurochem. 106(3):1184-9).

In some embodiments, the level of CDKN1A activity can be assessed by detecting the presence or absence of activity attenuating or inactivating mutations in CDKN1A protein or a gene encoding the CDKN1A protein. In some embodiments, the activity attenuating or inactivating mutation in the CDKNIA gene is a frameshift, nonsense, or missense mutation, particularly frameshift or nonsense mutation, or an activity attenuating or inactivating deletion of the gene encoding CDKN1A. In some embodiments, such mutations in CDKNIA can be assessed from information in The Cancer Genome Atlas (TCGA) dataset (see, e.g., Cazier et al., 2014, Nat Commun. 5:3756).

In some embodiments, as described herein, the cancer is assessed for presence of absence of activity-attenuating or inactivating mutation in the TP53 protein or the gene encoding the TP53 protein. In some embodiments, the activity attenuating or inactivating mutation in the TP53 gene is a frameshift, nonsense, or missense mutation, particularly frameshift or nonsense mutation, or an activity attenuating or inactivating deletion of the gene encoding TP53. Various mutations and deletions identified for TP53 are described in, among others, Hollstein et al., 1991, Science. 253(5015):49-53, and Schmitt et al., 2002, Cancer Cell. 1(3):289-98, all publications incorporated herein by reference. A database of TP53 mutations is available, for example, at the International Agency for Cancer Research (IARC) TP53 Database at world wide web at site p53.iarc.fr of the World Health Organization, version R18, April 2016 (see also Bouaoun et al., 2016, “TP53 Variations in Human Cancers: New Lessons from the IARC TP53 Database and Genomics Data,” Hum Mutat. 37(9):865-76, incorporated herein by reference).

In some embodiments, the panel of nucleic acid probes includes one or more nucleic acid probes for measuring expression levels of CDKN1A mRNA, and one or more probes for measuring expression levels of TP53 mRNA. In some embodiments, the panel of nucleic acid probes includes one or more nucleic acid probes for measuring expression levels of CDKN1A mRNA, and one or more nucleic acid probes for detecting activity-attenuating or inactivating mutations in TP53 gene or mRNA encoding the TP53 protein. In some embodiments, the panel of nucleic acid probes includes one or more nucleic acid probes for measuring expression levels of CDKN1A mRNA, one or more probes for measuring expression levels of TP53 mRNA, and one or more nucleic acid probes for detecting activity-attenuating or inactivating mutations in TP53 gene or mRNA encoding the TP53 protein. In some embodiments, any of the forgoing panel of nucleic acid probes can include nucleic acid probes for detecting activity attenuating or inactivating mutations in CDKN1A gene or the mRNA encoding the CDKN1A protein.

In some embodiments, the CDKN1A activity and/or TP53 expression/mutation status is determined for a biological sample of the cancer, particularly a biological sample of the cancer obtained from the patient being assessed or treated, as described herein. In some embodiments, the sample will typically be a sample of the cancer (or tumor) mass in the patient. The sample may be obtained from the primary tumor mass (if known and accessible) and/or from a metastatic tumor mass (if known and accessible). In some embodiments, such samples can be, but are not limited to, body fluid (e.g., blood, blood plasma, serum, peritoneal, lymph, interstitial, or urine), organs, tissues, fractions, and cells isolated from the subject or the patient being assessed. In some embodiments, the biological sample comprises, among others, a biopsy sample, an aspirate sample, lymphatic sample, or a blood sample containing the cancer. In some embodiments, the biological sample is a primary or cultured cells of the subject or patient. In some embodiments, the biological samples are frozen or fixed samples, such as tissue sections. In some embodiments, the biological sample can be analyzed as is, that is, without harvest and/or isolation of the target of interest. In some embodiments, the biological sample can be prepared by physical disruption, such as by sonication, homogenization, high speed blender, or by treatment with enzymes, fixatives, detergents, acids, denaturants, chaotropic agents, or other chemicals for preparing the sample for analysis.

In some embodiments, the sample is processed for detecting the protein of interest. In some embodiments, the biological sample can be processed to harvest and potentially isolate CDKN1A protein. In some embodiments, the protein samples can be bound to a support, such as membranes, beads, plastic surfaces, glass or derivatized glass, or fiber supports for detection using a binding agent. Preparation of samples and detection using binding agents, such as antibodies, are described in general references such as Current Protocols in Immunology, Coligan et al., eds., John Wiley & Sons (updates to 2015); Immunoassays: A Practical Approach, Gosling, ed., Oxford University Press (2000), incorporated herein by reference.

As discussed above, in some embodiments, CDKN1A mRNA levels is measured. In some embodiments, the sample containing the RNA can be used directly without much processing, or the sample can be processed to isolate and/or enrich for mRNA transcripts. The preparation of mRNA and isolation methods can be performed using techniques known in the art including but not limited to column and/or bead extraction methods. Kits for harvest and isolation of mRNA transcripts are also commercially available. As discussed above, in some embodiments, the mRNA can be amplified and its amplified product detected. Techniques for reverse transcription of mRNA transcripts into cDNA, amplification of mRNA and cDNA transcripts, and detection of such transcripts or their amplified products are also known in the art. CDKN1A mRNA transcripts, cDNA or amplified products of either can be detected by binding to a complementary nucleic acid probe that is specific for CDKN1A. The probe may be bound to a solid support such as an array, or a column, or a bead. Alternatively, the method may involve immobilizing the mRNA, cDNA or amplified product of either to a solid support and then interrogating the support with a nucleic acid probe that is specific for CDKN1A. The probe or the CDKN1A product can be labeled in a manner that allows its presence and location to be detected. For example, it may be labeled with a directly detectable label such as a fluorophore, a chemiluminescent label, a chromophore, a radiolabel, and the like.

In some embodiments, while the exemplary CDKN1A mRNA sequence described herein can be used to measure CDKN1A mRNA expression, the detection methods may be designed also to detect variants therefore which encode CDKN1A protein. Such variants may include degenerate nucleic acids which include alternative codons to those present in the wildtype allele, as discussed herein. In general, homologs and alleles typically will share at least 75% nucleotide identity and/or at least 90% amino acid identity to the aforementioned CDKN1A mRNA/cDNA and protein sequences, respectively. Thus, in addition to detecting the aforementioned mRNA/cDNA sequence, the methods provided herein may also detect nucleotide sequences having at least 65%, 70%, 75%, 80%, 85%, 90%, 91%, 92%, 93%, 94%, 95%, 96%, 97%, 98%, or at least 99% identity. In addition to detecting proteins having the aforementioned amino acid sequence, the methods provided herein may also detect proteins having amino acid sequences that share at least 91%, 92%, 93%, 94%, 95%, 96%, 97%, 98% or 99% amino acid identity.

The homology can be calculated using various publicly available software tools, for example software developed by NCBI (Bethesda, Md.) that can be obtained through the NCBI internet site. Exemplary tools include the BLAST software, also available at the NCBI internet site. Pairwise and ClustalW alignments (BLOSUM30 matrix setting) as well as Kyte-Doolittle hydropathic analysis can be obtained using the MacVector sequence analysis software (Oxford Molecular Group). It is to be understood that detection probes that are the Watson-Crick complements of the aforementioned nucleic acids may also be used in the detection methods provided herein.

In some embodiments, probes used to detect CDKN1A mRNA can be designed taking these parameters into consideration. Some embodiments involve detection of mRNA that encode functional CDKN1A proteins and/or detect functional CDKN1A protein. In these embodiments, while the detection targets may embrace wild-type as well as variants thereof, all such targets are or encode functional CDKN1A protein.

In some embodiments, hybridization between probes and targets are used under stringent conditions as is known and practiced in the art. Nucleic acid hybridization parameters are described in references, for examples, Molecular Cloning: A Laboratory Manual, J. Sambrook, et al., eds., 2nd Ed., Cold Spring Harbor Laboratory Press, Cold Spring Harbor, N.Y., 1989, or Current Protocols in Molecular Biology, F. M. Ausubel, et al., eds., John Wiley & Sons, Inc., New York. In some embodiments, stringent conditions refer, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA, where: SSC is 0.15M sodium chloride/0.015M sodium citrate, pH 7; SDS is sodium dodecyl sulphate; and EDTA is ethylenediaminetetracetic acid). After hybridization, the membrane upon which the DNA is transferred is washed, for example, in 2×SSC at room temperature and then at 0.1-0.5×SSC/0.1×SDS at temperatures up to 68° C. Other conditions and reagents sufficient to provide similar degree of stringency can also be used.

For detection of mutations in the targets of interest, such as CDKN1A or TP53, various techniques available to the skilled artisan can be used. In various embodiments, the presence or absence of a mutation can be determined by known DNA or RNA detection methods, for example, DNA sequencing, oligonucleotide hybridization, polymerase chain reaction (PCR) amplification with primers specific to the mutation, or protein detection methods, for example, immunoassays or biochemical assays to identify a mutated protein, such as mutated CDKN1A or TP53 protein. In some embodiments, the nucleic acid or RNA in a sample can be detected by any suitable methods or techniques of detecting gene sequences. Such methods include, but are not limited to, PCR, reverse transcriptase-PCR (RT-PCR), in situ PCR, in situ hybridization, Southern blot, Northern blot, sequence analysis, microarray analysis, or other DNA/RNA hybridization platforms (see, e.g., Taso et al., 2010, Lung Cancer 68(1):51-7). In particular, detection of mutations can use samples obtained non-invasively, such as cell free nucleic acid (e.g., cfDNA) from blood.

In some embodiments, mutations can be detected using various Next-Gen sequencing (NGS) techniques, particularly high-throughput NGS techniques. Exemplary NGS techniques include, among others, Polony sequencing (see, e.g., Shendure et al., 2005, Science 309(5741):1728-32), IonTorrent sequencing (see, e.g., Rusk, N., 2011, Nat Meth 8(1):44-44), pyrosequencing (see, e.g., Marguiles et al., 2005, Nature 437(7057):376-380), reversible dye sequencing with colony sequencing (Bentley et al., 2008, Nature 456(7218):53-59; Illumina, CA, USA), sequencing by ligation (e.g., SOLid systems of Applied Biosystems; Valouev et al., 2008, Genome Res. 18(7):1051-1063), high throughput rolling circle “nanoball” sequencing (see, e.g., Drmanac et al., 2010, Science 327 (5961):78-81; Porreca, G. J., 2010, Nature Biotech. 28 (1):43-44), and zero-mode wave guide based sequencing (see, e.g., Chin et al., 2013, Nat Methods 10(6):563-569); all publications incorporated herein by reference. In some embodiments, massively parallel sequencing of target genes, such as genes encoding CDKN1A or TP53 can be carried out to detect or identify presence or absence of mutations in the cancer being assessed for treatment with the ATR inhibitor.

In some embodiments, detection of point mutations in target nucleic acids can be accomplished by molecular cloning of the target nucleic acid molecules and sequencing the nucleic acid molecules using available techniques. Alternatively, amplification techniques such as PCR can be used to amplify target nucleic acid sequences directly from a genomic DNA preparation from a tumor tissue, cell sample, or cell free sample (e.g., cell free plasma from blood). The nucleic acid sequence of the amplified molecules can then be determined to identify mutations. Other methods of detecting mutations that can be used include, among others, ligase chain reaction, allele-specific PCR restriction fragment length polymorphism, single stranded conformation polymorphism analysis, mismatch detection proteins (e.g., GRIN2A or TRRAP), RNase protection (e.g., Winter et al., 1985, Proc. Natl. Acad. Sci. USA 82:7575-7579), enzymatic or chemical cleavage (Cotton et al., 1988, Proc. Natl. Acad. Sci. USA 85: 4397; Shenk et al., 1975, Proc. Natl. Acad. Sci. USA 72:989).

In some embodiments, mutations in nucleic acid molecules can also be detected by screening for alterations of the corresponding protein. For example, monoclonal antibodies immunoreactive with a target gene product can be used to screen a tissue, for example an antibody that is known to bind to a particular mutated position of the gene product (protein). For example, a suitable antibody may be one that binds to a deleted exon or that binds to a conformational epitope comprising a deleted portion of the target protein. Lack of cognate antigen would indicate a mutation. Such immunological assays can be accomplished using any convenient format known in the art, such as Western blot, immunohistochemical assay and ELISA.

General biological, biochemical, immunological and molecular biological methods applicable to the present disclosure, e.g., for detecting nucleic acids and proteins, are described in Sambrook et al., Molecular Cloning: A Laboratory Manual 2^{nd} Ed. (1989) Cold Spring Harbor Laboratory Press, Cold Spring Harbor, N.Y.; Current Protocols in Molecular Biology, Ausubel et al., ed., John Wiley & Sons (2015); Current Protocols in Immunology, Coligan, JE ed., John Wiley & Sons (2015); and Methods in Enzymology, Vol. 200, Abelson et al., ed., Academic Press (1991). All publications are incorporated herein by reference.

In some embodiments, the subjects or patients herein are afflicted with a cancer. In some embodiments, the subjects herein are human, also referred to as a patient. In some embodiments, the subjects are non-humans mammals which are appropriate for treatment with the ATR inhibitor, including for example domesticated mammals, such as dogs, cats, horses, or in some embodiments, other primates, such as chimpanzee and gorilla.

In some embodiments, the subject is diagnosed with a cancer. In some embodiments, the subject is diagnosed with cancer but not yet received any therapeutic treatments. In some embodiments, the subject is diagnosed with cancer, and has received one or more cancer therapies. In some embodiments, the treatment with the ATR inhibitor, in particular as a combination therapy, is a follow-on therapy, for example with disease progression following prior treatment. In some embodiments, the subject is diagnosed with advanced or late stage cancer. In some embodiments, the method of determining sensitivity of the cancer is used to follow progression of ATR inhibitor treatment, particularly the ATR inhibitor in a combination treatment, to assess any changes in sensitivity of the cancer to the treatment with the ATR inhibitor based on measuring CDKN1A activity and/or TP53 expression/mutation status.

In some embodiments, the cancers for screening and/or treatment according to the methods described herein are solid tumors, including primary tumors and metastatic tumors. In some embodiments, the cancer for the methods herein include: oral cancer, including buccal cavity cancer, lip cancer, tongue cancer, mouth cancer, and pharynx cancer; cardiac cancer, including sarcoma (e.g., angiosarcoma, fibrosarcoma, rhabdomyosarcoma, liposarcoma), myxoma, rhabdomyoma, fibroma, lipoma and teratoma; lung cancer, including bronchogenic carcinoma (e.g., squamous cell or epidermoid, undifferentiated small cell lung cancer, undifferentiated large cell lung cancer, adenocarcinoma), alveolar (bronchiolar) carcinoma, bronchial adenoma, sarcoma, lymphoma, chondromatous hamartoma, and mesothelioma; gastrointestinal cancer, including esophageal cancer (squamous cell carcinoma, larynx, adenocarcinoma, leiomyosarcoma, lymphoma), stomach cancer (e.g., carcinoma, lymphoma, leiomyosarcoma), pancreatic cancer (e.g., ductal adenocarcinoma, insulinoma, glucagonoma, gastrinoma, carcinoid tumors, vipoma), small bowel or small intestinal cancer (adenocarcinoma, lymphoma, carcinoid tumors, Karposi's sarcoma, leiomyoma, hemangioma, lipoma, neurofibroma, fibroma), large bowel or large intestinal cancer (e.g., adenocarcinoma, tubular adenoma, villous adenoma, hamartoma, leiomyoma), rectal cancer, colon cancer, and colorectal cancer; genitourinary tract cancer, including kidney cancer (adenocarcinoma, Wilm's tumor [nephroblastoma], lymphoma, leukemia), bladder and urethral cancer (squamous cell carcinoma, transitional cell carcinoma, adenocarcinoma), prostate cancer (adenocarcinoma, sarcoma), and testicular cancer (e.g., seminoma, teratoma, embryonal carcinoma, teratocarcinoma, choriocarcinoma, sarcoma, interstitial cell carcinoma, fibroma, fibroadenoma, adenomatoid tumors, lipoma); liver cancer, including hepatoma (hepatocellular carcinoma), cholangiocarcinoma, hepatoblastoma, angiosarcoma, hepatocellular adenoma, hemangioma, and biliary passages cancer; bone cancer, including osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system cancer, including skull cancer (e.g., osteoma, hemangioma, granuloma, xanthoma, osteitis deformans), meningial cancer (meningioma, meningiosarcoma, gliomatosis), brain cancer (e.g., astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, and sarcoma; gynecological cancer, including uterine cancer (endometrial carcinoma), cervical cancer (e.g., cervical carcinoma, pre-tumor cervical dysplasia), ovarian cancer (e.g., ovarian carcinoma (serous cystadenocarcinoma, mucinous cystadenocarcinoma, unclassified carcinoma), granulosa-thecal cell tumors, Sertoll-Leydig cell tumors, dysgerminoma, malignant teratoma), vulval cancer (squamous cell carcinoma, intraepithelial carcinoma, adenocarcinoma, fibrosarcoma, melanoma), vaginal cancer (clear cell carcinoma, squamous cell carcinoma, botryoid sarcoma (embryonal rhabdomyosarcoma), fallopian tube cancer (carcinoma), and breast cancer; hematologic cancer, including blood cancer (e.g., acute myeloid leukemia, chronic myeloid leukemia, acute lymphoblastic leukemia, chronic lymphocytic leukemia, myeloproliferative diseases, multiple myeloma, myelodysplastic syndrome), Hodgkin's disease, non-Hodgkin's lymphoma, malignant lymphoma, hairy cell lymphoma, and lymphoid disorders; skin cancer, including malignant melanoma, basal cell carcinoma, squamous cell carcinoma, Karposi's sarcoma, keratoacanthoma, moles dysplastic nevi, lipoma, angioma, dermatofibroma, keloids; thyroid gland cancer, including papillary thyroid carcinoma, follicular thyroid carcinoma, medullary thyroid carcinoma, multiple endocrine neoplasia type 2A, multiple endocrine neoplasia type 2B, familial medullary thyroid cancer, pheochromocytoma, and paraganglioma; and adrenal glands cancer, including: neuroblastoma.

In some embodiments, the cancers for screening and/or treatment according to the methods described herein include but are not limited to lung cancer (such as but not limited to non-small cell lung cancer (NSCLC) and small cell lung cancer), ovarian cancer, pancreatic cancer, head and neck cancer, esophageal cancer, endometrial cancer, breast cancer (e.g., ER⁺, HER2⁺ breast cancer, and triple negative breast cancer), colorectal cancer, testicular cancer, and cervical cancer.

In some embodiments, the cancers for screening and/or treatment according to the methods described herein include cancers having generally lower or reduced levels of CDKN1A expression as compared to other cancer types. In some embodiments, such cancers are selected from breast cancer, colorectal cancer, glioma/glioblastoma, liver cancer, lymphoma, ovarian cancer, prostate cancer, pancreatic cancer and testicular cancer.

ATR Inhibitors

In various embodiments herein, the ATR inhibitor as described herein inhibits the activity of ataxia telangiectasia mutated and rad3-related (ATR) kinase. ATR is a serine/threonine-specific protein kinase involved in sensing DNA damage, activating the DNA damage checkpoint, leading to cell cycle arrest, and triggering DNA damage repair. In some embodiments, the ATR inhibitor is a selective ATR inhibitor. In some embodiments, a selective ATR inhibitor refers to an ATR inhibitor which has a Ki/IC50 for ATR kinase but with minimal inhibitory activity against one or more of ATM and DNA-PK. In some embodiments, exemplary ATR inhibitors for the methods and uses of the present disclosure include those described in published patent applications WO2010/071837 and WO2014/089379, all of which are incorporated herein by reference. In some embodiments, the definition of chemical substituents in the following description of ATR inhibitor compounds uses those in WO2010/071837 and WO2014/089379.

In some embodiments, the ATR inhibitor is a compound of Formula IA:

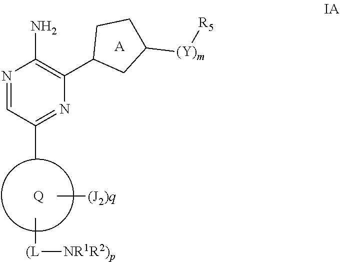

or a pharmaceutically acceptable salt thereof; wherein
Y is a C₁-C₁₀aliphatic chain wherein up to three methylene units of the aliphatic chain are optionally replaced with O, NR⁰, S, C(O) or S(O)₂;
Ring A is a 5 membered heteroaryl ring selected from

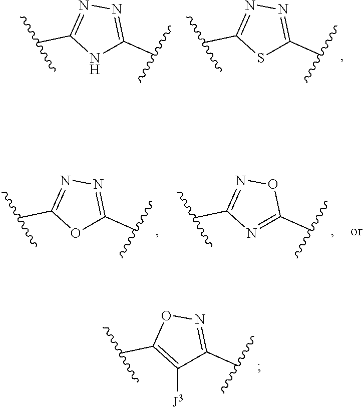

J³ is H or C₁-C₄alkyl, wherein 1 methylene unit of the alkyl group can optionally be replaced with O, NH, N(C₁-C₄alkyl), or S and optionally substituted with 1-3 halo;
Q is a 5-6 membered monocyclic aromatic ring containing 0-4 heteroatoms independently selected from nitrogen, oxygen, and sulfur; or an 8-10 membered bicyclic aromatic ring containing 0-6 heteroatoms independently selected from nitrogen, oxygen, and sulfur;
R⁵ is H; a 3-7 membered monocyclic fully saturated, partially unsaturated, or aromatic ring containing 0-4 heteroatoms independently selected from nitrogen, oxygen, and sulfur; an 8-10 membered bicyclic fully saturated, partially unsaturated, or aromatic ring containing 0-6 heteroatoms independently selected from nitrogen, oxygen, and sulfur; wherein R⁵ is optionally substituted with 1-5 J⁵ groups;
L is a C₁-C₄alkyl chain wherein up to two methylene units of the alkyl chain are optionally replaced with O, NR⁶, S, —C(O)—, —SO—, or —SO₂—;
R⁰ is H or C₁-C₆alkyl wherein one methylene unit of the alkyl chain can be optionally replaced with O, NH, N(C₁-C₄alkyl), or S;
R¹ is H or C₁-C₆alkyl;
R² is H, —(C₂-C₆alkyl)-Z or a 4-8 membered cyclic ring containing 0-2 nitrogen atoms; wherein said ring is bonded via a carbon atom and is optionally substituted with one occurrence of J^{Z};
or R¹ and R², taken together with the atom to which they are bound, form a 4-8 membered heterocyclic ring containing 1-2 heteroatoms selected from oxygen, nitrogen, and sulfur; wherein said heterocyclic ring is optionally substituted with one occurrence of J^{Z1};
J^{Z1} is halo, CN, C₁-C₈aliphatic, —(X)₁—CN, or —(X)ᵣ—Z, wherein said up to two methylene units of said C₁-C₈aliphatic can be optionally replaced with O, NR, S, P(O), C(O), S(O), or S(O)₂, wherein said C₁-C₈aliphatic is optionally substituted with halo, CN, or NO₂;
X is C₁-C₄alkyl;
each t, r and m is independently 0 or 1;
Z is —NR³R⁴;
R³ is H or C₁-C₂alkyl;
R⁴ is H or C₁-C₆alkyl;
or R³ and R⁴, taken together with the atom to which they are bound, form a 4-8 membered heterocyclic ring containing 1-2 heteroatoms selected from oxygen, nitrogen, and sulfur; wherein said ring is optionally substituted with one occurrence of J^{z};
R⁶ is H, or C₁-C₆alkyl;
J^{Z} is independently NH₂, NH(C₁-C₄aliphatic), N(C₁-C₄aliphatic)₂, halogen, C₁-C₄aliphatic, OH, O(C₁-C₄aliphatic), NO₂, CN, CO₂H, CO(C₁-C₄aliphatic), CO₂(C₁-C₄aliphatic), O(haloC₁-C₄aliphatic), or haloC₁-C₄aliphatic;
J⁵ is halo, oxo, CN, NO₂, X¹—R, or —(X¹)ₚ-Q⁴;
X¹ is C₁-C₁₀aliphatic; wherein 1-3 methylene units of said C₁-C₁₀aliphatic are optionally replaced with NR′—, —O—, —S—, C(═NR′), C(O), S(O)₂, or S(O), wherein X¹ is optionally and independently substituted with 1-4 occurrences of NH₂, NH(C₁-C₄aliphatic), N(C₁-C₄aliphatic)₂, halogen, C₁-C₄aliphatic, OH, O(C₁-C₄aliphatic), NO₂, CN, CO₂H, CO₂(C₁-C₄aliphatic), C(O)NH₂, C(O)NH(C₁-C₄aliphatic), C(O)N(C₁-C₄aliphatic)₂, SO(C₁-C₄aliphatic), SO₂(C₁-C₄aliphatic), SO₂NH(C₁-C₄aliphatic), NHC(O)(C₁-C₄aliphatic), N(C₁-C₄aliphatic)C(O)(C₁-C₄aliphatic), wherein said C₁-C₄aliphatic is optionally substituted with 1-3 occurrences of halo;
Q⁴ is a 3-8 membered saturated or unsaturated monocyclic ring having 0-4 heteroatoms independently selected from nitrogen, oxygen, and sulfur, or a 8-10 membered saturated or unsaturated bicyclic ring having 0-6 heteroatoms independently selected from nitrogen, oxygen, and sulfur; each Q⁴ is optionally substituted with 1-5 J^{Q4};
J^{Q4} is halo, CN, or C₁-C₄alkyl wherein up to 2 methylene units are optionally replaced with O, NR*, S, C(O), S(O), or S(O)₂;
R is H or C₁-C₄alkyl wherein said C₁-C₄alkyl is optionally substituted with 1-4 halo;
J² is halo; CN; a 5-6 membered aromatic or nonaromatic monocyclic ring having 0-3 heteroatoms selected from oxygen, nitrogen, and sulfur; or a C₁-C₁₀aliphatic group wherein up to 2 methylene units are optionally replaced with O, NR″, C(O), S, S(O), or S(O)₂; wherein said C₁-C₁₀aliphatic group is optionally substituted with 1-3 halo or CN; and said monocyclic ring is optionally substituted with 1-3 occurrences of halo; CN; a C₃-C₆cycloalkyl; a 3-7 membered heterocyclyl containing 0-2 heteroatoms selected from oxygen, nitrogen, and sulfur; or a C₁-C₄alkyl wherein up to one methylene unit of the alkyl chain is optionally replaced with O, NR″, or S; and wherein said C₁-C₄alkyl is optionally substituted with 1-3 halo;
q is 0, 1, or 2;
p is 0 or 1; and
R′, R″, and R* are each independently H, C₁-C₄alkyl, or is absent; wherein said C₁-C₄alkyl is optionally substituted with 1-4 halo.

In some embodiments, Ring A is:

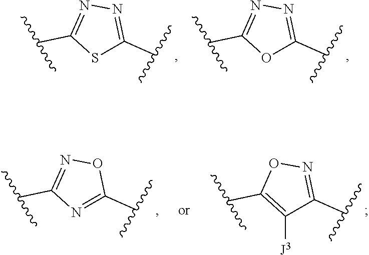

In some embodiments, Ring A is

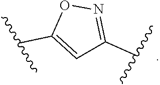 [SMILES: *c1cc(*)on1]

It should be understood that Ring A structures can be bound to the pyrazine ring in two different ways: as drawn, and the reverse (flipped). For example, when Ring A is

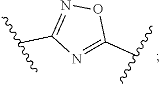 [SMILES: *c1noc(*)n1]

it can be bound to the pyrazine ring as shown below:

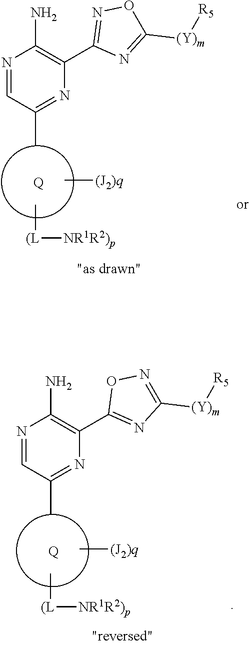

Similarly, when Ring A is

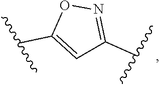 [SMILES: *c1cc(*)on1]

it can also be bound to the pyrazine ring in two ways —as drawn and reversed. In some embodiments, the Ring A structures are bound as drawn.

In some embodiments, J³ is H.

In some embodiments, J⁵ is a C₁-C₆aliphatic group, wherein up to 2 methylene units are optionally replaced with O or NR′R″ where each R′ and R″ is independently H or alkyl; or R′ and R″ taken together to form a 3-6 membered heterocyclic ring; NH₂, NH(C₁-C₄aliphatic), N(C₁-C₄aliphatic)₂, halogen, C₁-C₄aliphatic, OH, O(C₁-C₄aliphatic), NO₂, CN, CO₂H, CO(C₁-C₄aliphatic), CO₂(C₁-C₄aliphatic), O(halo C₁-C₄aliphatic), or halo C₁-C₄aliphatic.

In other embodiments, J² is halo, C₁-C₂alkyl optionally substituted with 1-3 fluoro, CN, or a C₁-C₄alkyl group wherein up to 2 methylene units are optionally replaced with S(O), S(O)₂, C(O), or NR′.

In some embodiments, J² is halo; CN; phenyl; oxazolyl; or a C₁-C₆aliphatic group, wherein up to 2 methylene units are optionally replaced with O, NR″, C(O), S, S(O), or S(O)₂; said C₁-C₆aliphatic group is optionally substituted with 1-3 fluoro or CN.

In some embodiments, the ATR inhibitor is a compound of Formula IIA:

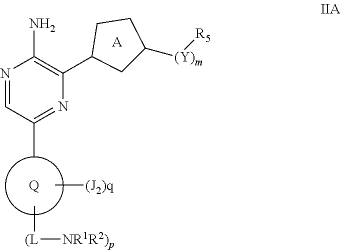

or a pharmaceutically acceptable salt thereof; wherein
Ring A is a 5 membered heteroaryl ring selected from

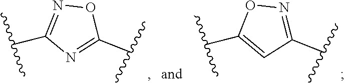 [SMILES: *c1noc(*)n1 ; *c1cc(*)on1]

Y is a C₁-C₄alkyl chain wherein one methylene unit of the alkyl chain is optionally replaced with —NR⁰—;
Q is a 5-6 membered monocyclic aromatic ring containing 0-4 heteroatoms independently selected from nitrogen, oxygen, and sulfur; or an 8-10 membered bicyclic aromatic ring containing 0-6 heteroatoms independently selected from nitrogen, oxygen, and sulfur;
R⁵ is 5-6 membered monocyclic aryl or heteroaryl ring having 0-3 heteroatoms independently selected from nitrogen, oxygen, and sulfur, R⁵ is optionally fused to a 5-6 membered aromatic ring containing 0-2 heteroatoms selected from nitrogen, oxygen, and sulfur; each R⁵ is optionally substituted with 1-5 J⁵ groups;
L is —C(O)— or —SO₂—;
R¹ is H, or C₁-C₆alkyl;
R⁰ is H or C₁-C₆alkyl;
R² is C₁-C₆alkyl, —(C₂-C₆alkyl)-Z or a 4-8 membered cyclic ring containing 0-2 nitrogen atoms, wherein said ring is bonded via a carbon atom and is optionally substituted with one occurrence of J^{Z};
or R¹ and R², taken together with the atom to which they are bound, form a 4-8 membered heterocyclic ring containing 1-2 heteroatoms selected from nitrogen, sulfur, and oxygen; wherein said heterocyclic ring is optionally substituted with one occurrence of J^{Z1};
J^{Z1} is (X)₁—CN, C₁-C₆alkyl or —(X)ᵣ—Z;
X is C₁-C₄alkyl;
each t, r and m is independently 0 or 1;
Z is —NR³R⁴;
R³ is H or C₁-C₂alkyl;
R⁴ is H or C₁-C₆alkyl;
or R³ and R⁴, taken together with the atom to which they are bound, form a 4-8 membered heterocyclic ring containing 1-2 nitrogen atoms; wherein said ring is optionally substituted with one occurrence of J^{Z};
J^{Z} is NH₂, NH(C₁-C₄aliphatic), N(C₁-C₄aliphatic)₂, halogen, C₁-C₄aliphatic, OH, O(C₁-C₄aliphatic), NO₂, CN, CO₂H, CO(C₁-C₄aliphatic), CO₂(C₁-C₄aliphatic), O(haloC₁-C₄aliphatic), or haloC₁-C₄aliphatic;
J⁵ is halogen, NO₂, CN, O(haloC₁-C₄aliphatic), haloC₁-C₄aliphatic, or a C₁-C₆aliphatic group wherein up to 2 methylene units are optionally replaced with C(O), O, or NR′;
J² is halo, CN, phenyl, oxazolyl, or a C₁-C₆aliphatic group wherein up to 2 methylene units are optionally replaced with O, NR″, C(O), S, S(O), or S(O)₂; said C₁-C₆aliphatic group is optionally substituted with 1-3 fluoro or CN;
R′ and R″ are each independently H or C₁-C₄alkyl;
q is 0, 1, or 2; and
p is 0 or 1.

In some embodiments, Q is phenyl or pyridyl.

In other embodiments, Y is a C₁-C₂alkyl chain wherein one methylene unit of the alkyl chain is optionally replaced with NR^{o}.

In some embodiments, the ATR inhibitor is selected from the compounds in Table 1:

TABLE I

| | |
|---|---|
| 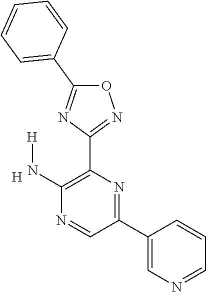 | IIA-1 |
| 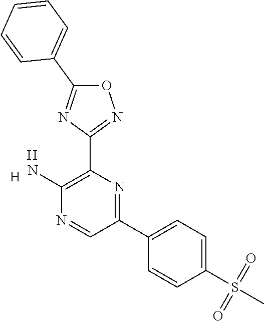 | IIA-2 |
| 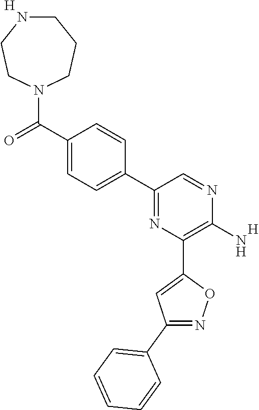 | IIA-3 |
| 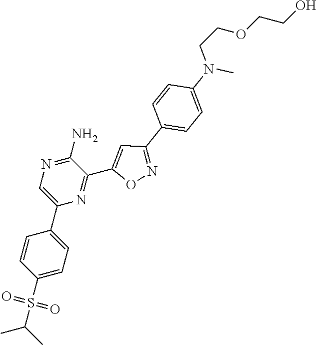 [SMILES: CC(C)S(=O)(=O)c1ccc(-c2cnc(N)c(-c3cc(-c4ccc(N(C)CCOCCO)cc4)no3)n2)cc1] | IIA-4 |
| 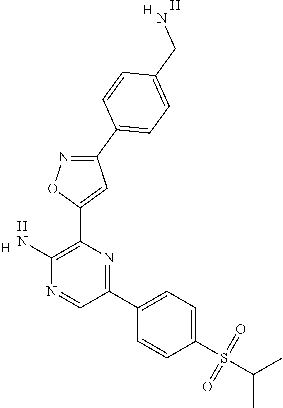 | IIA-5 |
| 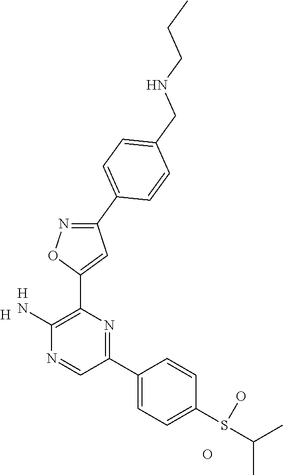 | IIA-6 |
| 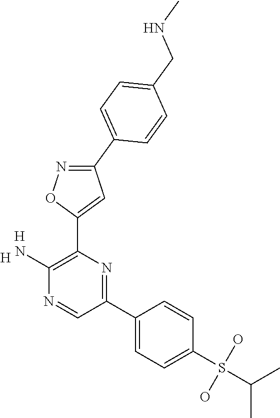 | IIA-7 |
| 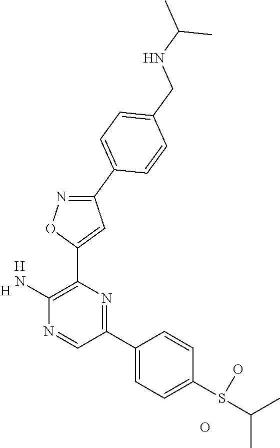 | IIA-8 |
| 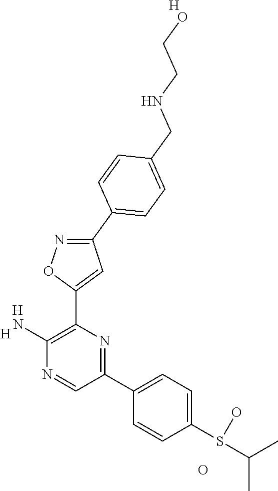 | IIA-9 |
| 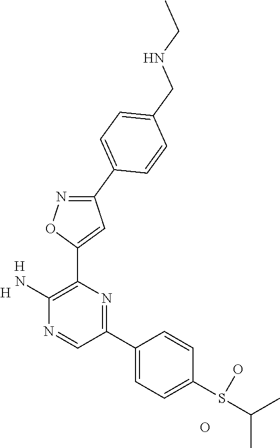 | IIA-10 |
| 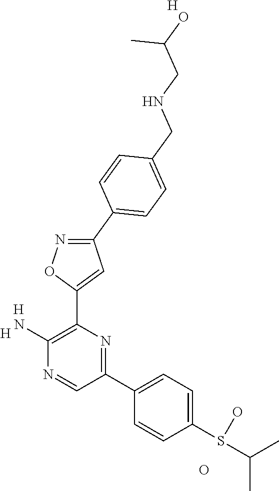 | IIA-11 |
| 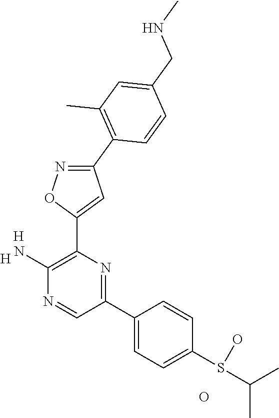 | IIA-12 |
| 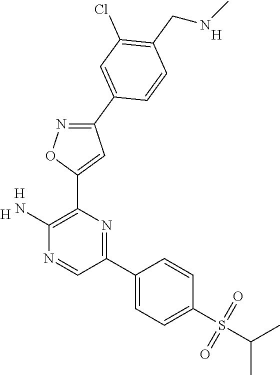 | IIA-13 |
| 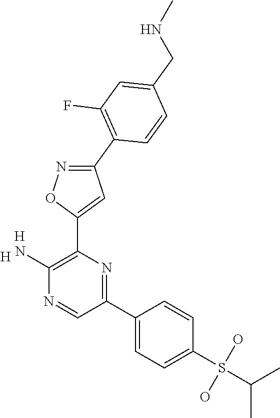 | IIA-14 |
| 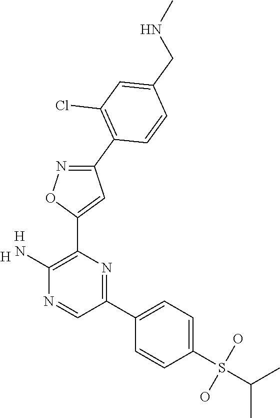 | IIA-15 |
| 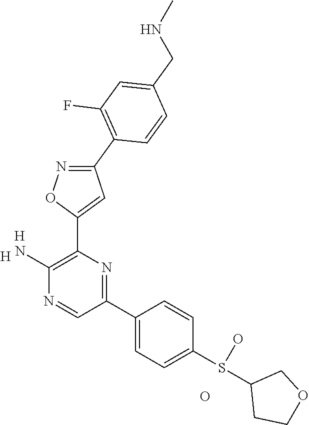 | IIA-16 |

In some embodiments, the ATR inhibitor is a compound of Formula IA-iii:

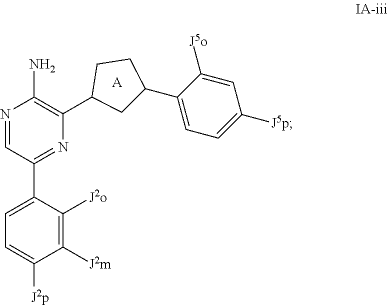

or a pharmaceutically acceptable salt thereof wherein;
Ring A is

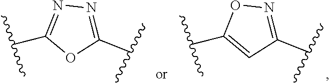 [SMILES: *c1nnc(*)o1 ; *c1cc(*)on1]

J⁵o is H, F, Cl, C₁-C₄aliphatic, O(C₁-C₃aliphatic), or OH;
J⁵p is

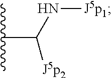

J⁵p1 is H, C₁-C₄aliphatic, oxetanyl, tetrahydrofuranyl, tetrahydropyranyl; wherein J⁵p2 is optionally substituted with 1-2 occurrences of OH or halo;
J⁵p2 is H, methyl, ethyl, CH₂F, CF₃, or CH₂OH;
J²o is H, CN, or SO₂CH₃;
J²m is H, F, Cl, or methyl; and
J²p is —SO₂(C₁-C₆alkyl), —SO₂(C₃-C₆cycloalkyl), —SO₂(4-6 membered heterocyclyl), —SO₂(C₁-C₄alkyl)N(C₁-C₄alkyl)₂, or —SO₂(C₁-C₄alkyl)-(4-6 membered heterocyclyl), wherein said heterocyclyl contains 1 heteroatom selected from the group consisting of O, N, and S; and wherein said J²p is optionally substituted with 1-3 occurrences halo, OH, or O(C₁-C₄alkyl).

In some embodiments, Ring A is

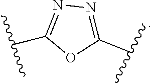 [SMILES: *c1nnc(*)o1]

In other embodiments, Ring A is

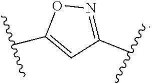 [SMILES: *c1cc(*)on1]

In some embodiments, the ATR inhibitor is a compound of the following structure (IIA-7):

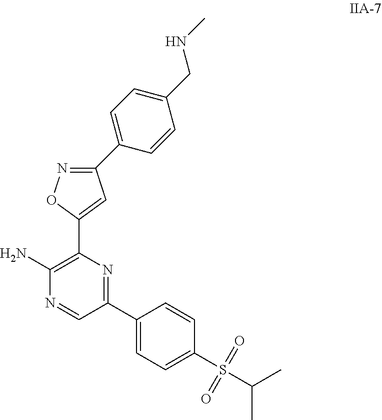 [SMILES: CNCc1ccc(-c2cc(-c3nc(-c4ccc(S(=O)(=O)C(C)C)cc4)cnc3N)on2)cc1]

or a pharmaceutically acceptable salt thereof.

In some embodiments, the ATR inhibitor is a compound of Formula I:

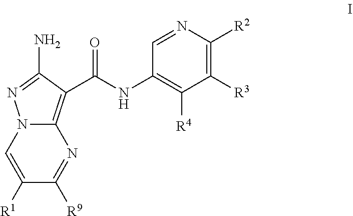

or a pharmaceutically acceptable salt thereof, wherein:
R¹ is independently selected from —C(J¹)₂CN, halo, -(L)ₖ-W, and M;
R⁹ is independently selected from H, —C(J¹)₂CN, halo, -(L)ₖ-W, and M;
J¹ is independently selected from H and C₁-C₂alkyl; or two occurrences of J¹, together with the carbon atom to which they are attached, form a 3-4 membered optionally substituted carbocyclic ring;
k is 0 or 1;
M and L are a C₁-C₈aliphatic, wherein up to three methylene units are optionally replaced with —O—, —NR—, —C(O)—, or —S(O)ₙ—, each M and L′ is optionally substituted with 0-3 occurrences of J^{LM};
J^{LM} is independently selected from halo, —CN, and a C₁-C₄aliphatic chain wherein up to two methylene units of the aliphatic chain are optionally replaced with —O—, —NR—, —C(O)—, or —S(O)ₙ—;
W is independently selected from a 3-7 membered fully saturated, partially unsaturated, or aromatic monocyclic ring having 0-3 heteroatoms selected from oxygen, nitrogen and sulfur; and a 7-12 membered fully saturated, partially unsaturated, or aromatic bicyclic ring having 0-5 heteroatoms selected from oxygen, nitrogen, and sulfur; wherein W is optionally substituted with 0-5 occurrences of J^{W};
J^{W} is independently selected from —CN, halo, —CF₃; a C₁-C₄aliphatic wherein up to two methylene units are optionally replaced with —O—, —NR—, —C(O)—, or —S(O)ₙ—; and a 3-6 membered non-aromatic ring having 0-2 heteroatoms selected from oxygen, nitrogen, and sulfur; or two occurrences of J^{W} on the same atom, together with atom to which they are joined, form a 3-6 membered ring having 0-2 heteroatoms selected from oxygen, nitrogen, and sulfur; or two occurrences of J^{W}, together with W, form a 6-10 membered saturated or partially unsaturated bridged ring system;
R² is independently selected from H; halo; —CN; NH₂; a C₁-C₂alkyl optionally substituted with 0-3 occurrences of fluoro; and a C₁-C₃aliphatic chain wherein up to two methylene units of the aliphatic chain are optionally replaced with —O—, —NR—, —C(O)—, or —S(O)ₙ;
R³ is independently selected from H; halo; C₁-C₄alkyl optionally substituted with 1-3 occurrences of halo; C₃-C₄cycloalkyl; 3-4 membered heterocyclyl; —CN; and a C₁-C₃aliphatic chain wherein up to two methylene units of the aliphatic chain are optionally replaced with —O—, —NR—, —C(O)—, or —S(O)ₙ;
R⁴ is independently selected from Q¹ and a C₁-C₁₀aliphatic chain wherein up to four methylene units of the aliphatic chain are optionally replaced with —O—, —NR—, —C(O)—, or —S(O)ₙ—; each R⁴ is optionally substituted with 0-5 occurrences of J^{Q}; or
R³ and R⁴, taken together with the atoms to which they are bound, form a 5-6 membered aromatic or non-aromatic ring having 0-2 heteroatoms selected from oxygen, nitrogen and sulfur; the ring formed by R³ and R⁴ is optionally substituted with 0-3 occurrences of J^{Z};
Q¹ is independently selected from a 3-7 membered fully saturated, partially unsaturated, or aromatic monocyclic ring, the 3-7 membered ring having 0-3 heteroatoms selected from oxygen, nitrogen and sulfur; and an 7-12 membered fully saturated, partially unsaturated, or aromatic bicyclic ring having 0-5 heteroatoms selected from oxygen, nitrogen, and sulfur;
J^{z} is independently selected from C₁-C₆aliphatic, ═O, halo, and →O;
J^{Q} is independently selected from —CN; halo; ═O; Q²; and a C₁-C₈aliphatic chain wherein up to three methylene units of the aliphatic chain are optionally replaced with —O—, —NR—, —C(O)—, or —S(O)ₙ—; each occurrence of J^{Q} is optionally substituted by 0-3 occurrences of J^{R}; or two occurrences of J^{Q} on the same atom, taken together with the atom to which they are joined, form a 3-6 membered ring having 0-2 heteroatoms selected from oxygen, nitrogen, and sulfur; wherein the ring formed by two occurrences of J^{Q} is optionally substituted with 0-3 occurrences of J^{X}; or two occurrences of J^{Q}, together with Q′, form a 6-10 membered saturated or partially unsaturated bridged ring system;
Q² is independently selected from a 3-7 membered fully saturated, partially unsaturated, or aromatic monocyclic ring having 0-3 heteroatoms selected from oxygen, nitrogen, and sulfur; and an 7-12 membered fully saturated, partially unsaturated, or aromatic bicyclic ring having 0-5 heteroatoms selected from oxygen, nitrogen, and sulfur;
J^{R} is independently selected from —CN; halo; ═O; →O; Q³; and a C₁-C₆aliphatic chain wherein up to three methylene units of the aliphatic chain are optionally replaced with —O—, —NR—, —C(O)—, or —S(O)ₙ—; each J^{R} is optionally substituted with 0-3 occurrences of J^{T}; or two occurrences of J^{R} on the same atom, together with the atom to which they are joined, form a 3-6 membered ring having 0-2 heteroatoms selected from oxygen, nitrogen, and sulfur; wherein the ring formed by two occurrences of J^{R} is optionally substituted with 0-3 occurrences of J^{X}; or two occurrences of J^{R}, together with Q², form a 6-10 membered saturated or partially unsaturated bridged ring system;
Q³ is a 3-7 membered fully saturated, partially unsaturated, or aromatic monocyclic ring having 0-3 heteroatoms selected from oxygen, nitrogen, or sulfur; or an 7-12 membered fully saturated, partially unsaturated, or aromatic bicyclic ring having 0-5 heteroatoms selected from oxygen, nitrogen, and sulfur;
J^{X} is independently selected from-CN; ═O; halo; and a C₁-C₄aliphatic chain, wherein up to two methylene units of the aliphatic chain are optionally replaced with —O—, —NR—, —C(O)—, or —S(O)ₙ—;
J^{T} is independently selected from halo, —CN; →O; ═O; —OH; a C₁-C₆aliphatic chain wherein up to two methylene units of the aliphatic chain are optionally replaced with —O—, —NR—, —C(O)—, or —S(O)ₙ—; and a 3-6 membered non-aromatic ring having 0-2 heteroatoms selected from oxygen, nitrogen, and sulfur; each occurrence of J^{T} is optionally substituted with 0-3 occurrences of J^{M}; or two occurrences of J^{T} on the same atom, together with the atom to which they are joined, form a 3-6 membered ring having 0-2 heteroatoms selected from oxygen, nitrogen, and sulfur; or two occurrences of J^{T}, together with Q³, form a 6-10 membered saturated or partially unsaturated bridged ring system;
J^{M} is independently selected from halo and C₁-C₆aliphatic;
n is 0, 1 or 2; and
R is independently selected from H and C₁-C₄aliphatic.

In some embodiments, the compound is represented by formula I, wherein R⁹ is H.

In some embodiments, the compound is represented by formula I, wherein R⁹ is M. In some embodiments, the compound is represented by formula I, wherein M is a C₁-C₈aliphatic wherein up to three methylene units are optionally replaced with —O— or —NR—. In some aspects, the compound is represented by formula I, wherein M is C₁-C₄alkyl, —(C₁-C₄alkyl)O(C₁-C₃aliphatic), —(C₁-C₃alkyl)OH, —O(C₁-C₄alkyl)N(C₁-C₂alkyl)₂, —NH(C₁-C₄alkyl), or —(C₁-C₄alkyl)NH(C₁-C₄alkyl). In some embodiments, the compound is represented by formula I, wherein M is C₁-C₄alkyl.

In some embodiments, the compound is represented by formula I, wherein J^{LM} is halo.

In some embodiments, the compound is represented by formula I, wherein R⁹ is -(L)ₖ-W.

In some embodiments, the compound is represented by formula I, wherein k is 1. In some embodiments, the compound is represented by formula I, wherein k is 0.

In some embodiments, the compound is represented by formula I, wherein L is a C₁-C₈aliphatic wherein up to three methylene units are optionally replaced with —O— or —NR—. In some embodiments, the compound is represented by formula I, wherein L is —O—, —O(C₁-C₄aliphatic)-, or —NR(C₁-C₃alkyl)-.

In some embodiments, the compound is represented by formula I, wherein W is a 3-7 membered fully saturated, partially unsaturated, or aromatic monocyclic ring having 0-3 heteroatoms selected from oxygen, nitrogen and sulfur. In some embodiments, the compound is represented by formula I, wherein W is a 3-7 membered heterocyclyl. In some embodiments, the compound is represented by formula I, wherein W is independently selected from pyrrolidinyl, piperidinyl, piperazinyl, oxetanyl, and azetidinyl.

In some embodiments, the compound is represented by formula I, wherein W is a 7-12 membered fully saturated, partially unsaturated, or aromatic bicyclic ring having 0-5 heteroatoms selected from oxygen, nitrogen, and sulfur. In some embodiments, the compound is represented by formula I, wherein W is octahydropyrrolo[1,2-a]pyrazine.

In some embodiments, the compound is represented by formula I, wherein J^{W} is selected form C₁-C₃alkyl or CF₃. In some embodiments, the compound is represented by formula I, wherein two occurrences of J^{W} on the same atom, together with atom to which they are joined, form a 3-6 membered ring having 0-2 heteroatoms selected from oxygen, nitrogen, and sulfur. In some embodiments, the compound is represented by formula I, wherein the ring formed by the two occurrences of J^{W} on the same atom is oxetanyl.

In some embodiments, the ATR inhibitor is a compound of Formula I-A:

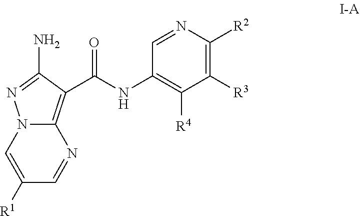

or a pharmaceutically acceptable salt or prodrug thereof, wherein:
R¹ is independently selected from fluoro, chloro, and —C(J¹)₂CN;
J¹ is independently selected from H and C₁-C₂alkyl; or
   two occurrences of J¹, together with the carbon atom to which they are attached, form a 3-4 membered optionally substituted carbocyclic ring;
R² is independently selected from H; halo; —CN; NH₂; a C₁-C₂alkyl optionally substituted with 0-3 occurrences of fluoro; and a C₁₋₃ aliphatic chain wherein up to two methylene units of the aliphatic chain are optionally replaced with —O—, —NR—, —C(O)—, or —S(O)ₙ;
R³ is independently selected from H; halo; C₁-C₄alkyl optionally substituted with 1-3 occurrences of halo; C₃-C₄cycloalkyl; —CN; and a C₁-C₃aliphatic chain wherein up to two methylene units of the aliphatic chain are optionally replaced with —O—, —NR—, —C(O)—, or —S(O)ₙ;
R⁴ is independently selected from Q¹ and a C₁-C₁₀aliphatic chain wherein up to four methylene units of the aliphatic chain are optionally replaced with —O—, —NR—, —C(O)—, or —S(O)ₙ—; each R⁴ is optionally substituted with 0-5 occurrences of J^{Q}; or
R³ and R⁴, taken together with the atoms to which they are bound, form a 5-6 membered aromatic or non-aromatic ring having 0-2 heteroatoms selected from oxygen, nitrogen and sulfur; the ring formed by R³ and R⁴ is optionally substituted with 0-3 occurrences of J^{Z};
Q¹ is independently selected from a 3-7 membered fully saturated, partially unsaturated, or aromatic monocyclic ring, the 3-7 membered ring having 0-3 heteroatoms selected from oxygen, nitrogen and sulfur; and a 7-12 membered fully saturated, partially unsaturated, or aromatic bicyclic ring having 0-5 heteroatoms selected from oxygen, nitrogen, and sulfur;
J^{z} is independently selected from C₁-C₆aliphatic, ═O, halo, and →O;
J^{Q} is independently selected from —CN; halo; ═O; Q²; and a C₁-C₈aliphatic chain wherein up to three methylene units of the aliphatic chain are optionally replaced with —O—, —NR—, —C(O)—, or —S(O)ₙ—; each occurrence of J^{Q} is optionally substituted by 0-3 occurrences of J^{R}; or two occurrences of J^{Q} on the same atom, taken together with the atom to which they are joined, form a 3-6 membered ring having 0-2 heteroatoms selected from oxygen, nitrogen, and sulfur; wherein the ring formed by two occurrences of J^{Q} is optionally substituted with 0-3 occurrences of J^{X}; or two occurrences of J^{Q}, together with Q¹, form a 6-10 membered saturated or partially unsaturated bridged ring system;
Q² is independently selected from a 3-7 membered fully saturated, partially unsaturated, or aromatic monocyclic ring having 0-3 heteroatoms selected from oxygen, nitrogen, and sulfur; and an 7-12 membered fully saturated, partially unsaturated, or aromatic bicyclic ring having 0-5 heteroatoms selected from oxygen, nitrogen, and sulfur;
J^{R} is independently selected from —CN; halo; ═O; →O; Q³; and or a C₁-C₆aliphatic chain wherein up to three methylene units of the aliphatic chain are optionally replaced with —O—, —NR—, —C(O)—, or —S(O)ₙ; each J^{R} is optionally substituted with 0-3 occurrences of J^{T}; or two occurrences of J^{R} on the same atom, together with the atom to which they are joined, form a 3-6 membered ring having 0-2 heteroatoms selected from oxygen, nitrogen, and sulfur; wherein the ring formed by two occurrences of J^{R} is optionally substituted with 0-3 occurrences of J^{X}; or two occurrences of J^{R}, together with Q², form a 6-10 membered saturated or partially unsaturated bridged ring system;
Q³ is a 3-7 membered fully saturated, partially unsaturated, or aromatic monocyclic ring having 0-3 heteroatoms selected from oxygen, nitrogen, or sulfur; or an 7-12 membered fully saturated, partially unsaturated, or aromatic bicyclic ring having 0-5 heteroatoms selected from oxygen, nitrogen, and sulfur;
J^{X} is independently selected from-CN; ═O; halo; and a C₁-C₄aliphatic chain wherein up to two methylene units of the aliphatic chain are optionally replaced with —O—, —NR—, —C(O)—, or —S(O)ₙ—;
J^{T} is independently selected from halo, —CN; →O; ═O; —OH; a C₁-C₆aliphatic chain wherein up to two methylene units of the aliphatic chain are optionally replaced with —O—, —NR—, —C(O)—, or —S(O)ₙ—; and a 3-6 membered non-aromatic ring having 0-2 heteroatoms selected from oxygen, nitrogen, and sulfur; each occurrence of J^{T} is optionally substituted with 0-3 occurrences of J^{M}; or two occurrences of J^{T} on the same atom, together with the atom to which they are joined, form a 3-6 membered ring having 0-2 heteroatoms selected from oxygen, nitrogen, and sulfur; or two occurrences of J^{T}, together with Q³, form a 6-10 membered saturated or partially unsaturated bridged ring system;
J^{M} is independently selected from halo and C₁-C₆aliphatic;
n is 0, 1 or 2; and
R is independently selected from H and C₁-C₄aliphatic.

In some embodiments, the ATR inhibitor is a compound of Formula I-A:

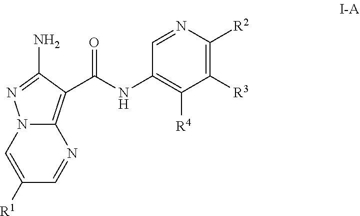

or a pharmaceutically acceptable salt thereof, wherein:
R¹ is independently selected from fluoro, chloro, and —C(J¹)₂CN;
J¹ is independently selected from H and C₁-C₂alkyl; or
   two occurrences of J¹, together with the carbon atom to which they are attached, form a 3-4 membered optionally substituted carbocyclic ring;
R² is independently selected from H; halo; —CN; NH₂; a C₁-C₂alkyl optionally substituted with 0-3 occurrences of fluoro; and a C₁-C₃aliphatic chain wherein up to two methylene units of the aliphatic chain are optionally replaced with —O—, —NR—, —C(O)—, or —S(O)ₙ;
R³ is independently selected from H; halo; C₁-C₄alkyl optionally substituted with 1-3 occurrences of halo; C₃-C₄cycloalkyl; —CN; and a C₁-C₃aliphatic chain wherein up to two methylene units of the aliphatic chain are optionally replaced with —O—, —NR—, —C(O)—, or —S(O)ₙ;
R⁴ is independently selected from Q¹ and a C₁-C₁₀aliphatic chain wherein up to four methylene units of the aliphatic chain are optionally replaced with —O—, —NR—, —C(O)—, or —S(O)ₙ—; each R⁴ is optionally substituted with 0-5 occurrences of J^{Q}; or
R³ and R⁴, taken together with the atoms to which they are bound, form a 5-6 membered non-aromatic ring having 0-2 heteroatoms selected from oxygen, nitrogen and sulfur; the ring formed by R³ and R⁴ is optionally substituted with 0-3 occurrences of J^{Z};
Q¹ is independently selected from a 3-7 membered fully saturated, partially unsaturated, or aromatic monocyclic ring, the 3-7 membered ring having 0-3 heteroatoms selected from oxygen, nitrogen and sulfur; and an 7-12 membered fully saturated, partially unsaturated, or aromatic bicyclic ring having 0-5 heteroatoms selected from oxygen, nitrogen, and sulfur;
J^{z} is independently selected from C₁-C₆aliphatic, ═O, halo, and →O;
J^{Q} is independently selected from —CN; halo; ═O; Q²; and a C₁-C₈aliphatic chain wherein up to three methylene units of the aliphatic chain are optionally replaced with —O—, —NR—, —C(O)—, or —S(O)ₙ—; each occurrence of J^{Q} is optionally substituted by 0-3 occurrences of J^{R}; or
   two occurrences of J^{Q} on the same atom, taken together with the atom to which they are joined, form a 3-6 membered ring having 0-2 heteroatoms selected from oxygen, nitrogen, and sulfur; wherein the ring formed by two occurrences of J^{Q} is optionally substituted with 0-3 occurrences of J^{X}; or
   two occurrences of J^{Q}, together with Q¹, form a 6-10 membered saturated or partially unsaturated bridged ring system;
Q² is independently selected from a 3-7 membered fully saturated, partially unsaturated, or aromatic monocyclic ring having 0-3 heteroatoms selected from oxygen, nitrogen, and sulfur; and a 7-12 membered fully saturated, partially unsaturated, or aromatic bicyclic ring having 0-5 heteroatoms selected from oxygen, nitrogen, and sulfur;
J^{R} is independently selected from —CN; halo; ═O; →O; Q³; and a C₁-C₆aliphatic chain wherein up to three methylene units of the aliphatic chain are optionally replaced with —O—, —NR—, —C(O)—, or —S(O)ₙ—; each J^{R} is optionally substituted with 0-3 occurrences of J^{T}; or
   two occurrences of J^{R} on the same atom, together with the atom to which they are joined, form a 3-6 membered ring having 0-2 heteroatoms selected from oxygen, nitrogen, and sulfur; wherein the ring formed by two occurrences of J^{R} is optionally substituted with 0-3 occurrences of J^{X}; or two occurrences of J^{R}, together with Q², form a 6-10 membered saturated or partially unsaturated bridged ring system;
Q³ is a 3-7 membered fully saturated, partially unsaturated, or aromatic monocyclic ring having 0-3 heteroatoms selected from oxygen, nitrogen, and sulfur; or a 7-12 membered fully saturated, partially unsaturated, or aromatic bicyclic ring having 0-5 heteroatoms selected from oxygen, nitrogen, and sulfur;
J^{X} is independently selected from —CN; halo; and a C₁-C₄aliphatic chain wherein up to two methylene units of the aliphatic chain are optionally replaced with —O—, —NR—, —C(O)—, or —S(O)ₙ—;
J^{T} is independently selected from —CN; ═O; —OH; a C₁-C₆aliphatic chain wherein up to two methylene units of the aliphatic chain are optionally replaced with —O—, —NR—, —C(O)—, or —S(O)ₙ—; and a 3-6 membered non-aromatic ring having 0-2 heteroatoms selected from oxygen, nitrogen, and sulfur; each occurrence of J^{T} is optionally substituted with 0-3 occurrences of J^{M}; or
   two occurrences of J^{T} on the same atom, together with the atom to which they are joined, form a 3-6 membered ring having 0-2 heteroatoms selected from oxygen, nitrogen, and sulfur; or two occurrences of J^{T}, together with Q³, form a 6-10 membered saturated or partially unsaturated bridged ring system;
J^{M} is independently selected from halo and C₁-C₆aliphatic;
n is 0, 1 or 2; and
R is independently selected from H and C₁-C₄aliphatic.

In some embodiments, the ATR inhibitor is a compound of Formula I-A:

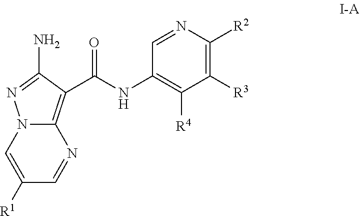

or a pharmaceutically acceptable salt or prodrug thereof, wherein:
R¹ is independently selected from fluoro, chloro, and —C(J¹)₂CN;
J¹ is independently selected from H and C₁-C₂alkyl; or
   two occurrences of J¹, together with the carbon atom to which they are attached, form an optionally substituted 3-4 membered carbocyclic ring;
R² is independently selected from H; chloro; NH₂; and a C₁-C₂alkyl optionally substituted with fluoro;
R³ is independently selected from H; chloro; fluoro; C₁-C₄alkyl optionally substituted with 1-3 occurrences of halo; C₃-C₄cycloalkyl; and —CN;
R⁴ is independently selected from Q¹ and a C₁-C₁₀aliphatic chain wherein up to three methylene units of the aliphatic chain are optionally replaced with —O—, —NR—, or —S—; each R⁴ is optionally substituted with 0-5 occurrences of J^{Q}; or
R³ and R⁴, taken together with the atoms to which they are bound, form a 5-6 membered non-aromatic ring having 0-2 heteroatoms selected from oxygen, nitrogen and sulfur; the ring formed by R³ and R⁴ is optionally substituted with 0-3 occurrences of J^{Z};
Q¹ is independently selected from a 3-7 membered fully saturated, partially unsaturated, or aromatic monocyclic ring having 0-3 heteroatoms selected from oxygen, nitrogen and sulfur; and an 7-12 membered fully saturated, partially unsaturated, or aromatic bicyclic ring; having 0-5 heteroatoms selected from oxygen, nitrogen, and sulfur;
J^{z} is independently selected from C₁-C₆aliphatic, ═O, halo, and →O;
J^{Q} is independently selected from halo; ═O; Q²; and a C₁-C₈aliphatic chain wherein up to two methylene units of the aliphatic chain are optionally replaced with —O—, —NR—, —S—, —C(O)—, or —S(O)ₙ—; each occurrence of r is optionally substituted by 0-3 occurrences of J^{R}; or
   two occurrences of r on the same atom, taken together with the atom to which they are joined, form a 3-6 membered ring having 0-2 heteroatoms selected from oxygen, nitrogen, and sulfur; wherein the ring formed by two occurrences of r is optionally substituted with 0-3 occurrences of J^{X}; or
   two occurrences of J^{Q}, together with Q¹, form a 6-10 membered saturated or partially unsaturated bridged ring system;
Q² is independently selected from a 3-7 membered fully saturated, partially unsaturated, or aromatic monocyclic ring having 0-3 heteroatoms selected from oxygen, nitrogen, and sulfur; and an 8-12 membered fully saturated, partially unsaturated, or aromatic bicyclic ring having 0-5 heteroatoms selected from oxygen, nitrogen, and sulfur;
J^{R} is independently selected from halo; ═O; →O; a 3-7 membered fully saturated, partially unsaturated, or aromatic monocyclic ring having 0-3 heteroatoms selected from oxygen, nitrogen, and sulfur; and a C₁-C₄aliphatic chain wherein up to two methylene units of the aliphatic chain are optionally replaced with —O—, —NR—, —S—, —C(O)—, or —S(O)ₙ—; each J^{R} is optionally substituted with 0-3 occurrences of J^{T}; or
   two occurrences of J^{R} on the same atom, together with the atom to which they are joined, form a 3-6 membered ring having 0-2 heteroatoms selected from oxygen, nitrogen, and sulfur; wherein the ring formed by two occurrences of J^{R} is optionally substituted with 0-3 occurrences of J^{X}; or two occurrences of J^{R}, together with Q², form a 6-10 membered saturated or partially unsaturated bridged ring system;
J^{X} is independently selected from halo and or a C₁-C₄aliphatic chain wherein up to two methylene units of the aliphatic chain are optionally replaced with —O—, —NR—, —S—, —C(O)—, or —S(O)ₙ—; or
J^{T} is independently selected from a C₁-C₆aliphatic and a 3-6 membered non-aromatic ring having 0-2 heteroatoms selected from oxygen, nitrogen, and sulfur; each occurrence of J^{T} is optionally substituted with 0-3 occurrences of J^{M};
J^{M} is independently selected from halo and C₁-C₆aliphatic;
n is 1 or 2; and
R is independently selected from H and C₁-C₄aliphatic.

In some embodiments, the ATR inhibitor is a compound of structural formula I or I-A, wherein R¹ is fluoro. In some embodiments, the compound is represented by structural formula I or I-A, wherein R¹ is —CH₂CN. In some embodiments, R¹ is —CH(C₁₋₂ alkyl)CN. In some embodiments, the compound is represented by structural formula I or I-A, wherein R¹ is C(CH₃)₂CN. In some embodiments, the compound is represented by structural formula I or I-A, wherein R′ is chloro.

In some embodiments, the compound is represented by structural formula I or I-A, wherein R² is independently selected from —CF₃, —NH(C₁-C₂alkyl), chloro, or H. In some embodiments, the compound is represented by structural formula I or I-A, wherein R² is H. In some embodiments, the compound is represented by structural formula I or I-A, wherein R² is -chloro.

In some embodiments, the compound is represented by structural formula I or I-A, wherein R³ is independently selected from H, chloro, fluoro, CHF₂, —CN, cyclopropyl, and C₁-C₄alkyl. In some embodiments, the compound is represented by structural formula I or I-A, wherein R³ is independently selected from H, chloro, and fluoro. In some embodiments, the compound is represented by structural formula I or I-A, wherein R³ is H. In some embodiments, the compound is represented by structural formula I or I-A, wherein R³ is —O(C₁-C₂alkyl). In some embodiments, the compound is represented by structural formula I or I-A, wherein R³ is chloro. In some embodiments, the compound is represented by structural formula I or I-A, wherein R³ is fluoro.

In some embodiments, the ATR inhibitor is a compound of structural formula I or I-A, wherein R⁴ is independently selected from:

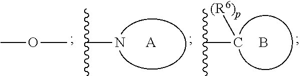

and —CH₂—R⁷,
wherein:
—O— is substituted with one J^{Q};
Ring A is independently selected from a 3-7 membered fully saturated, partially unsaturated, or aromatic monocyclic ring having 1-3 heteroatoms selected from oxygen, nitrogen and sulfur; and an 7-12 membered fully saturated, partially unsaturated, or aromatic bicyclic ring having 1-5 heteroatoms selected from oxygen, nitrogen, and sulfur;
Ring B is independently selected from a 3-7 membered fully saturated, partially unsaturated, or aromatic monocyclic ring having 0-3 heteroatoms selected from oxygen, nitrogen and sulfur; and an 7-12 membered fully saturated, partially unsaturated, or aromatic bicyclic ring having 0-5 heteroatoms selected from oxygen, nitrogen, and sulfur;
R⁶ is H;
R⁷ is independently selected from H and a C₁-C₈aliphatic chain wherein up to three methylene units of the aliphatic chain are optionally replaced with —O—, —NR—, —S—, —C(O)—, or —S(O)ₙ—;
p is 0 or 1; and
n is 0, 1, or 2.

In some embodiments, the ATR inhibitor is a compound of structural formula I or I-A, wherein R⁴ is Ring A, which is represented by the structure:

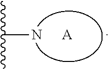

In some embodiments, the ATR inhibitor is a compound of structural formula I or I-A, wherein Ring A is a is a 3-7 membered fully saturated, partially unsaturated, or aromatic monocyclic ring having 1-3 heteroatoms selected from oxygen, nitrogen and sulfur. In some embodiments, the compound is represented by structural formula I or I-A, wherein Ring A is a 4-6 membered heterocyclyl. In some embodiments, the compound is represented by structural formula I or I-A, wherein Ring A is a 3-7 membered heterocyclyl. In some embodiments, the compound is represented by structural formula I or I-A, wherein Ring A is independently selected from pyrrolidinyl, piperidinyl, azepanyl, pyrazolidinyl, isoxazolidinyl, oxazolidinyl, thiazolidinyl, imidazolidinyl, piperazinyl, morpholinyl, thiomorpholinyl, 1,3-oxazinanyl, 1,3-thiazinanyl, dihydropyridinyl, dihydroimidazolyl, 1,3-tetrahydropyrimidinyl, dihydropyrimidinyl, 1,4-diazepanyl, 1,4-oxazepanyl, 1,4-thiazepanyl, and azetidinyl. In some embodiments, the compound is represented by structural formula I or I-A, wherein Ring A is independently selected from piperidinyl, piperazinyl, 1,4-diazepanyl, thiomorpholinyl, pyrrolidinyl, azepanyl, and morpholinyl. In some embodiments, the compound is represented by structural formula I or I-A, wherein Ring A is independently selected from piperazinyl and piperidinyl.

In some embodiments, the ATR inhibitor is a compound of structural formula I or I-A, wherein Ring A is a 5-membered heteroaryl. In some embodiments, the compound is represented by structural formula I or I-A, wherein Ring A is independently selected from pyrrolyl, imidazolyl, pyrazolyl, 1,2,3-triazolyl, and 1,2,4-triazolyl. In some embodiments, the compound is represented by structural formula I or I-A, wherein Ring A is independently selected from pyrazolyl and imidazolyl.

In some embodiments, the ATR inhibitor is a compound of structural formula I or I-A, wherein Ring A is a 7-12 membered fully saturated, partially unsaturated, or aromatic bicyclic ring having 1-5 heteroatoms selected from oxygen, nitrogen, and sulfur. In some embodiments, the compound is represented by structural formula I or I-A, wherein Ring A is independently selected from octahydropyrrolo[1,2-a]pyrazinyl, 5,6,7,8-tetrahydroimidazo[1,2-a]pyridinyl, octahydro-1H-pyrazino [1,2-a] pyrazinyl, 5,6, 7,8-tetrahydroimidazo [1,5-a] pyrazinyl, 2,5-diazabicyclo[4.1.0], and octahydropyrazino [2,1-c] [1,4]oxazinyl.

In some embodiments, the ATR inhibitor is a compound of structural formula I or I-A, wherein when R⁴ is Ring A, J^{Q} is C₁-C₈aliphatic chain wherein up to two methylene units of the aliphatic chain are optionally replaced with —O—, —NR—, or —C(O)—. In some embodiments, the compound is represented by structural formula I or I-A, wherein when R⁴ is Ring A, J^{Q} is a C₁-C₆aliphatic chain wherein up to two methylene units of the aliphatic chain are optionally replaced with —O—, —NR—, or —C(O)—. In some embodiments, the compound is represented by structural formula I or I-A, wherein when R⁴ is Ring A, J^{Q} is independently selected from —O—, —C(O)—, —S(O)₂—, C₁-C₄alkyl, —(C₀-C₄alkyl)NH₂, —(C₀-C₄alkyl)NH(C₁-C₄alkyl), —(C₀-C₄alkyl)N(C₁-C₄alkyl)₂, —(C₀-C₄alkyl)OH, —(C₀-C₄alkyl)O(C₁-C₄alkyl), —C(O)OH, —S(O)₂N(C₁-C₃alkyl)-, —C(O)(C₁-C₄alkyl)-, —(O)C(C₁-C₄alkyl)N(C₁-C₂alkyl)₂ or —C(O)O(C₁-C₄alkyl). In some embodiments, the compound is represented by structural formula I or I-A, wherein when R⁴ is Ring A, J^{Q} is independently selected from —C(O)—, C₁-C₄alkyl, —(C₀-C₄alkyl)NH₂, —(C₀-C₄alkyl)NH(C₁-C₄alkyl), —(C₀-C₄alkyl)N(C₁-C₄alkyl)₂, —(C₀-C₄alkyl)OH, —(C₀-C₄alkyl)O(C₁-C₄alkyl), —C(O)OH, and —C(O)O(C₁-C₄alkyl). In still other embodiments, the compound is represented by structural formula I or I-A, wherein when R⁴ is Ring A, J^{Q} is C₁-C₄alkyl. In some embodiments, the compound is represented by structural formula I or I-A, wherein when R⁴ is Ring A, J^{Q} is C₁-C₄alkyl, —O—, or —C(O)—.

In some embodiments, when R⁴ is Ring A, then J^{Q} is Q².

In some embodiments, the ATR inhibitor is a compound of structural formula I or I-A, wherein when R⁴ is Ring A, Q² is a 3-7 membered heterocyclyl or carbocyclyl; the heterocyclyl having 1-3 heteroatoms selected from oxygen, nitrogen, and sulfur. In some embodiments, the compound is represented by structural formula I or I-A, wherein when R⁴ is Ring A, Q² is independently selected from selected from oxetanyl, tetrahydropyranyl, tetrahydrofuranyl, cyclopropyl, azetidinyl, pyrrolidinyl, piperazinyl, cyclobutyl, thiomorpholinyl, and morpholinyl. In some embodiments, the compound is represented by structural formula I or I-A, wherein when R⁴ is Ring A, Q² is independently selected from oxetanyl, tetrahydropyranyl, and tetrahydrofuranyl. In some embodiments, the compound is represented by structural formula I or I-A, wherein when R⁴ is Ring A, then Q² is oxetanyl.

In some embodiments, the ATR inhibitor is represented by structural formula I or I-A, wherein when R⁴ is Ring A, Q² is a 7-12 membered fully saturated, partially unsaturated, or aromatic bicyclic ring having 0-5 heteroatoms selected from oxygen, nitrogen, and sulfur. In some embodiments, the compound is represented by structural formula I or I-A, wherein when R⁴ is Ring A, Q² is an 8-12 membered fully saturated, partially unsaturated, or aromatic bicyclic ring having 0-5 heteroatoms selected from oxygen, nitrogen, and sulfur. In some embodiments, the compound is represented by structural formula I or I-A, wherein when R⁴ is Ring A, Q² is independently selected from 5,6,7,8-tetrahydroimidazo[1,5-a]pyrazinyl and 5,6,7,8-tetrahydroimidazo[1,2-a]pyrazinyl.

In some embodiments, the ATR inhibitor is represented by structural formula I or I-A, wherein two occurrences of J^{Q}, together with Ring A, form a bridged ring system.

In some embodiments, the ATR inhibitor is represented by structural formula I or I-A, wherein when R⁴ is Ring A, J^{Q} is =0.

In some embodiments, the ATR inhibitor is represented by structural formula I or I-A, wherein when R⁴ is Ring A, J^{R} is a 3-6 membered heterocyclyl having 1-3 heteroatoms selected from oxygen, nitrogen, and sulfur. In some embodiments, the compound is represented by structural formula I or I-A, wherein when R⁴ is Ring A, J^{R} is independently selected from oxetanyl, piperadinyl, azetidinyl, piperazinyl, pyrrolidinyl, and morpholinyl. In some embodiments, the compound is represented by structural formula I or I-A, wherein when R⁴ is Ring A, J^{R} is a piperazinyl.

In some embodiments, the ATR inhibitor is represented by structural formula I or I-A, wherein when R⁴ is Ring A, J^{R} is independently selected from halo, ═O, —OH, C₁-C₄alkyl, —(C₀-C₄alkyl)N(C₁-C₄alkyl)₂, and —(C₀-C₄ alkyl)O(C₁-C₄alkyl).

In some embodiments, the ATR inhibitor is represented by structural formula I or I-A, wherein when R⁴ is Ring A, two occurrences of J^{R} on the same atom, together with the atom to which they are joined, form a 3-6 membered aromatic or non-aromatic ring having 0-2 heteroatoms selected from oxygen, nitrogen, or sulfur. In other embodiments, the compound is represented by structural formula I or I-A, wherein when R⁴ is Ring A, J^{R} is independently selected from oxetanyl and azetidinyl.

In some embodiments, the ATR inhibitor is represented by structural formula I or I-A, wherein two occurrences of J^{R}, together with Ring A, form a bridged ring system.

In some embodiments, the ATR inhibitor is represented by structural formula I or I-A, wherein J^{T} is a 3-6 membered non-aromatic ring having 0-2 heteroatoms selected from oxygen, nitrogen, and sulfur. In some embodiments, the compound is represented by structural formula I or I-A, wherein J^{T} is oxytanyl. In another embodiment, J^{T} is a C₁-C₆aliphatic. In some embodiments, J^{T} is methyl.

In some embodiments, the ATR inhibitor is a compound represented by structural formula I or I-A, wherein R⁴ is Ring B, which is represented by the structure:

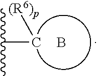

In some embodiments, the ATR inhibitor is represented by structural formula I or I-A, wherein p is 1.

In some embodiments, the ATR inhibitor is represented by structural formula I or I-A, wherein when p is 1, Ring B is a 3-7 membered cycloaliphatic or heterocyclyl ring having 1-2 heteroatoms selected from oxygen, nitrogen and sulfur. In some embodiments, the compound is represented by structural formula I or I-A, wherein when p is 1, Ring B is independently selected from selected from cyclopropyl, cyclobutyl, cyclopentyl, cyclohexyl, cycloheptyl, pyrrolidinyl, piperidinyl, azepanyl, pyrazolidinyl, isoxazolidinyl, oxazolidinyl, thiazolidinyl, imidazolidinyl, piperazinyl, morpholinyl, thiomorpholinyl, 1,3-oxazinanyl, 1,3-thiazinanyl, dihydropyridinyl, dihydroimidazolyl, 1,3-tetrahydropyrimidinyl, dihydropyrimidinyl, 1,4-diazepanyl, 1,4-oxazepanyl, 1,4-thiazepanyl, 1,2,3,6-tetrahydropyridine, and azetidinyl. In some embodiments, the compound is represented by structural formula I or I-A, wherein Ring B is piperidinyl.

In some embodiments, the ATR inhibitor is a compound of structural formula I or I-A, wherein when R⁴ is Ring B, J^{Q} is —C(O)— or C₁-C₄alkyl. In some embodiments, the compound is represented by structural formula I or I-A, wherein when R⁴ is Ring B, J^{Q} is C₁-C₄alkyl.

In some embodiments, the ATR inhibitor is a compound of structural formula I or I-A, wherein when R⁴ is Ring B, J^{Q} is Q². In some embodiments, when R⁴ is Ring B, the compound is represented by structural formula I or I-A, wherein Q² is independently selected from Q² is independently selected from oxetanyl, tetrahydropyranyl, tetrahydrofuranyl, cyclopropyl, azetidinyl, pyrrolidinyl, piperazinyl, piperidinyl, cyclobutyl, thiomorpholinyl, and morpholinyl. In some embodiments, the compound is represented by structural formula I or I-A, wherein when R⁴ is Ring B, Q² is oxetanyl.

In some embodiments, the ATR inhibitor is represented by structural formula I or I-A, wherein p is 0.

In some embodiments, the ATR inhibitor is represented by structural formula I or I-A, wherein when p is 0, Ring B is independently selected from phenyl, pyridinyl, pyrazinyl, pyrimidinyl, tetrahydropyridinyl, pyridizinyl, and pyrazolyl. In some embodiments, the compound is represented by structural formula I or I-A, wherein when p is 0, Ring B is imidazolyl. In some embodiments, the compound is represented by structural formula I or I-A, wherein when p is 0, Ring B is independently selected from phenyl and pyridinyl.

In some embodiments, the ATR inhibitor is represented by structural formula I or I-A, wherein R⁴ is —CH₂—(R⁷). In some embodiments, the compound is represented by structural formula I or I-A, wherein R¹ is H.

In some embodiments, the ATR inhibitor is represented by structural formula I or I-A, wherein R³ and R⁴, taken together with the atoms to which they are bound, form a 5-6 membered non-aromatic ring having 0-2 heteroatoms selected from oxygen.

In some embodiments, the present invention is a compound represented by structural formula I or I-A, wherein J^{z} is independently selected from →O or C₁-C₄alkyl.

In some embodiments, the ATR inhibitor is a compound of structural formula I and I-A, wherein the compounds are represented in Table 2.

TABLE 2

| | |
|---|---|
| 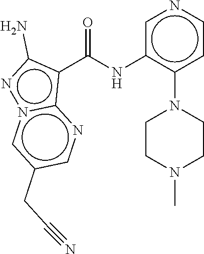 | I-N-82 |
| 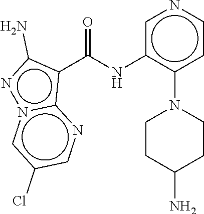 | I-N-82 |
| 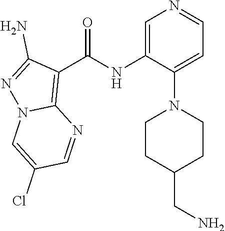 [SMILES: NCC1CCN(c2ccncc2NC(=O)c2c(N)nn3cc(Cl)cnc23)CC1] | I-N-91 |
| 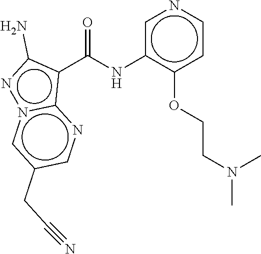 | I-O-24 |
| 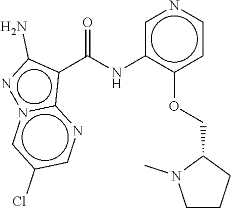 | I-O-50 |
| 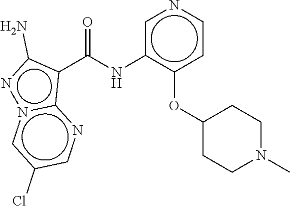 | I-O-64 |
| 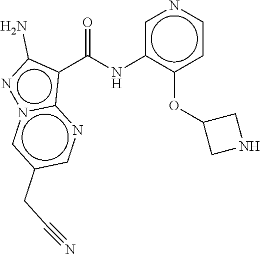 | I-O-82 |
| 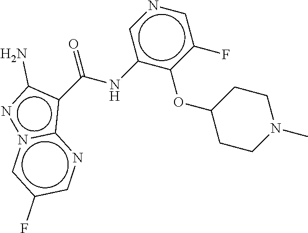 | I-O-89 |
| 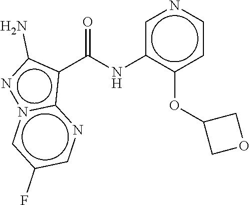 | I-O-92 |
| 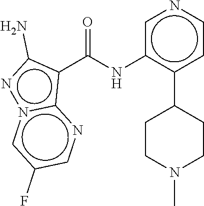 | I-C-1 |
| 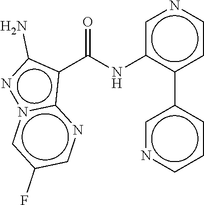 | I-C-15 |
| 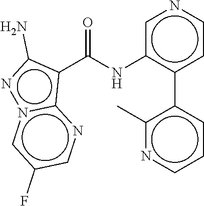 | I-C-20 |
| 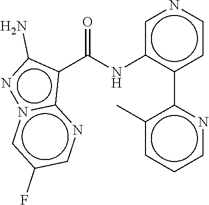 | I-C-36 |
| 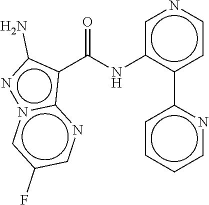 | I-C-60 |
| 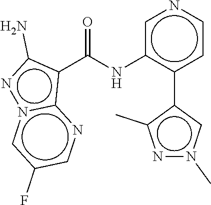 | I-C-63 |
| 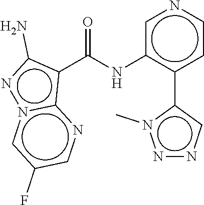 | I-C-72 |
| 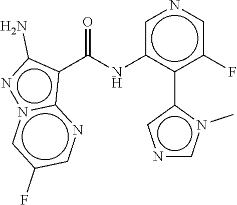 | I-C-79 |
| 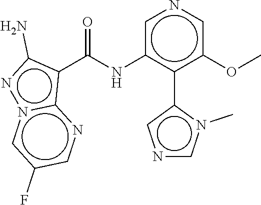 | I-C-84 |

In some embodiments, the ATR inhibitor has the following structure:

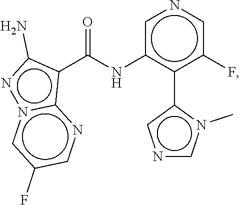

or a pharmaceutically acceptable salt thereof.

In some embodiments, the ATR inhibitor has the following structure:

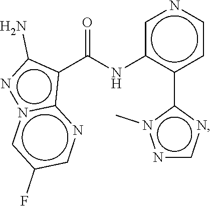

or a pharmaceutically acceptable salt thereof.

In some embodiments, the ATR inhibitor is a compound of structural formula I-B:

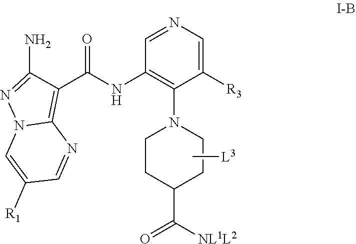

or a pharmaceutically acceptable salt thereof, wherein:
R¹ is independently selected from fluoro, chloro, and —C(J¹)₂CN;
J¹ is independently selected from H and C₁-C₂alkyl; or two occurrences of J¹, together with the carbon atom to which they are attached, form an optionally substituted 3-4 membered carbocyclic ring;
R³ is independently selected from H; chloro; fluoro; C₁-C₄alkyl optionally substituted with 1-3 occurrences of halo; C₃-C₄cycloalkyl; —CN; and a C₁-C₃aliphatic chain wherein up to two methylene units of the aliphatic chain are optionally replaced with —O—, —NR—, —C(O)—, or —S(O)ₙ;
L¹ is H; a 3-7 membered aromatic or non-aromatic ring having 0-2 heteroatoms selected from oxygen nitrogen and sulfur; and a C₁-C₆aliphatic chain wherein up to two methylene units of the aliphatic chain are optionally replaced with —O—, —NR—, —C(O)—, or —S(O)ₙ; each L¹ is optionally substituted with C₁-C₄aliphatic; —CN; halo; —OH; or a 3-6 membered non-aromatic ring having 0-2 heteroatoms selected from oxygen, nitrogen, and sulfur;
L² is H; a 3-7 membered aromatic or non-aromatic ring having 0-2 heteroatoms selected from oxygen nitrogen and sulfur; or a C₁-C₆aliphatic chain wherein up to two methylene units of the aliphatic chain are optionally replaced with —O—, —NR—, —C(O)—, or —S(O)ₙ; each L² is optionally substituted with C₁-C₄aliphatic; —CN; halo; —OH; or a 3-6 membered non-aromatic ring having 0-2 heteroatoms selected from oxygen, nitrogen, and sulfur; or
L¹ and L², together with the nitrogen to which they are attached, form a Ring D; Ring D is optionally substituted with 0-5 occurrences of J^{G};
Ring D is independently selected from a 3-7 membered heterocyclyl ring having 1-2 heteroatoms selected from oxygen, nitrogen and sulfur; and an 7-12 membered fully saturated or partially unsaturated bicyclic ring having 1-5 heteroatoms selected from oxygen, nitrogen, and sulfur;
J^{G} is independently selected from halo; —N(R^{o})₂; a 3-6 membered carbocycyl; a 3-6 membered heterocyclyl having 1-2 heteroatoms selected from oxygen nitrogen, and sulfur; or a C₁-C₄alkyl chain wherein up to two methylene units of the alkyl chain are optionally replaced with —O—, —NR—, —C(O)—, or —S(O)ₙ; each J^{G} is optionally substituted with 0-2 occurrences of J^{K}; or two occurrences of J^{G} on the same atom, together with the atom to which they are joined, form a 3-6 membered ring having 0-2 heteroatoms selected from oxygen, nitrogen, and sulfur; or two occurrences of J^{G}, together with Ring D, form a 6-10 membered saturated or partially unsaturated bridged ring system;
J^{K} is a 3-7 membered aromatic or non-aromatic ring having 0-2 heteroatoms selected from oxygen, nitrogen, and sulfur;
L³ is independently selected from H; chloro; fluoro; C₁-C₄alkyl optionally substituted with 1-3 occurrences of halo; —CN; and a C₁-C₃aliphatic chain wherein up to two methylene units of the aliphatic chain are optionally replaced with —O—, —NR—, —C(O)—, or —S(O)ₙ;
n is 0, 1, or 2; and
R and R^{o} are H or C₁-C₄alkyl.

In some embodiments, the ATR inhibitor is a compound of structural Formula I-B:

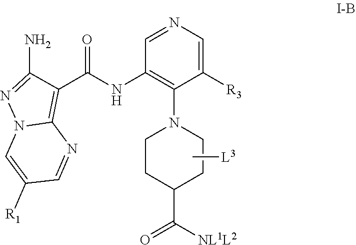

or a pharmaceutically acceptable salt thereof, wherein:
R¹ is independently selected from fluoro, chloro, and —C(J¹)₂CN;
J¹ is independently selected from H and C₁-C₂alkyl; or two occurrences of J′, together with the carbon atom to which they are attached, form an optionally substituted 3-4 membered carbocyclic ring;
R³ is independently selected from H; chloro; fluoro; C₁-C₄alkyl optionally substituted with 1-3 occurrences of halo; C₃-C₄cycloalkyl; —CN; and a C₁-C₃aliphatic chain wherein up to two methylene units of the aliphatic chain are optionally replaced with —O—, —NR—, —C(O)—, or —S(O)ₙ;
L¹ is H; a 3-7 membered aromatic or non-aromatic ring having 0-2 heteroatoms selected from oxygen nitrogen and sulfur; or a C₁-C₆aliphatic chain wherein up to two methylene units of the aliphatic chain are optionally replaced with —O—, —NR—, —C(O)—, or —S(O)ₙ; each L′ is optionally substituted with C₁-C₄aliphatic; —CN; halo; —OH; or a 3-6 membered non-aromatic ring having 0-2 heteroatoms selected from oxygen, nitrogen, and sulfur;
L² is H; a 3-7 membered aromatic or non-aromatic ring having 0-2 heteroatoms selected from oxygen nitrogen and sulfur; or a C₁-C₆aliphatic chain wherein up to two methylene units of the aliphatic chain are optionally replaced with —O—, —NR—, —C(O)—, or —S(O)ₙ; each L² is optionally substituted with C₁-C₄aliphatic; —CN; halo; —OH; or a 3-6 membered non-aromatic ring having 0-2 heteroatoms selected from oxygen, nitrogen, and sulfur; or
L¹ and L², together with the nitrogen to which they are attached, form a Ring D; Ring D is optionally substituted with 0-5 occurrences of J^{G};
Ring D is independently selected from a 3-7 membered heterocyclyl ring having 1-2 heteroatoms selected from oxygen, nitrogen and sulfur; or an 7-12 membered fully saturated or partially unsaturated bicyclic ring having 1-5 heteroatoms selected from oxygen, nitrogen, and sulfur;
J^{G} is independently selected from halo; —CN; —N(R^{o})₂; a 3-6 membered carbocycyl; a 3-6 membered heterocyclyl having 1-2 heteroatoms selected from oxygen nitrogen, and sulfur; or a C₁-C₄alkyl chain wherein up to two methylene units of the alkyl chain are optionally replaced with —O—, —NR—, —C(O)—, or —S(O)ₙ; each J^{G} is optionally substituted with 0-2 occurrences of J^{K}; or
   two occurrences of J^{G} on the same atom, together with the atom to which they are joined, form a 3-6 membered ring having 0-2 heteroatoms selected from oxygen, nitrogen, and sulfur; two occurrences of J^{G}, together with Ring D, form a 6-10 membered saturated or partially unsaturated bridged ring system;
J^{K} is a 3-7 membered aromatic or non-aromatic ring having 0-2 heteroatoms selected from oxygen, nitrogen, and sulfur;
n is 0, 1, or 2; and
R and R^{o} are H or C₁-C₄alkyl.

In some embodiments, the ATR inhibitor is a compound of structural Formula I-B:

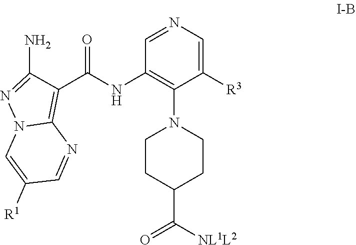

or a pharmaceutically acceptable salt thereof, wherein:
R¹ is independently selected from fluoro, chloro, and —C(J¹)₂CN;
J¹ is independently selected from H and C₁-C₂alkyl; or
   two occurrences of J′, together with the carbon atom to which they are attached, form an optionally substituted 3-4 membered carbocyclic ring;
R³ is independently selected from H; chloro; fluoro; C₁-C₄alkyl optionally substituted with 1-3 occurrences of halo; C₃-C₄cycloalkyl; —CN; and a C₁-C₃aliphatic chain wherein up to two methylene units of the aliphatic chain are optionally replaced with —O—, —NR—, —C(O)—, or —S(O)ₙ;
L¹ is H; a 3-7 membered aromatic or non-aromatic ring having 0-2 heteroatoms selected from oxygen nitrogen and sulfur; and a C₁-C₆aliphatic chain wherein up to two methylene units of the aliphatic chain are optionally replaced with —O—, —NR—, —C(O)—, or —S(O)ₙ; each L′ is optionally substituted with C₁-C₄aliphatic; —CN; halo; —OH; or a 3-6 membered non-aromatic ring having 0-2 heteroatoms selected from oxygen, nitrogen, and sulfur;
L² is H; a 3-7 membered aromatic or non-aromatic ring having 0-2 heteroatoms selected from oxygen nitrogen and sulfur; or a C₁-C₆aliphatic chain wherein up to two methylene units of the aliphatic chain are optionally replaced with —O—, —NR—, —C(O)—, or —S(O)ₙ; each L² is optionally substituted with C₁-C₄aliphatic; —CN; halo; —OH; or a 3-6 membered non-aromatic ring having 0-2 heteroatoms selected from oxygen, nitrogen, and sulfur; or
L¹ and L², together with the nitrogen to which they are attached, form a Ring D; Ring D is optionally substituted with 0-5 occurrences of J^{G};
Ring D is independently selected from a 3-7 membered heterocyclyl ring having 1-2 heteroatoms selected from oxygen, nitrogen and sulfur; or an 7-12 membered fully saturated or partially unsaturated bicyclic ring having 1-5 heteroatoms selected from oxygen, nitrogen, and sulfur;
J^{G} is independently selected from halo; →O; —CN; —N(R^{o})₂; a 3-6 membered carbocycyl; a 3-6 membered heterocyclyl having 1-2 heteroatoms selected from oxygen nitrogen, and sulfur; or a C₁-C₄alkyl chain wherein up to two methylene units of the alkyl chain are optionally replaced with —O—, —NR—, —C(O)—, or —S(O)ₙ; each J^{G} is optionally substituted with 0-2 occurrences of J^{K}; or two occurrences of J^{G} on the same atom, together with the atom to which they are joined, form a 3-6 membered ring having 0-2 heteroatoms selected from oxygen, nitrogen, and sulfur; or two occurrences of J^{G}, together with Ring D, form a 6-10 membered saturated or partially unsaturated bridged ring system;
J^{K} is a 3-7 membered aromatic or non-aromatic ring having 0-2 heteroatoms selected from oxygen, nitrogen, and sulfur;
n is 0, 1, or 2; and
R and R^{o} are H or C₁-C₄alkyl.

In some embodiments, the ATR inhibitor is a compound of structural Formula I-B:

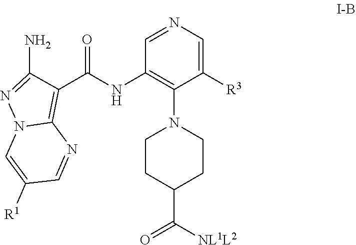

or a pharmaceutically acceptable salt thereof, wherein:
R¹ is independently selected from fluoro, chloro, and —C(J¹)₂CN;
J¹ is independently selected from H and C₁-C₂alkyl; or
   two occurrences of J¹, together with the carbon atom to which they are attached, form an optionally substituted 3-4 membered carbocyclic ring;
R³ is independently selected from H; chloro; fluoro; C₁-C₄alkyl optionally substituted with 1-3 occurrences of halo; C₃-C₄cycloalkyl; —CN; and a C₁-C₃aliphatic chain wherein up to two methylene units of the aliphatic chain are optionally replaced with —O—, —NR—, —C(O)—, or —S(O)ₙ;
L¹ is H; a 3-7 membered aromatic or non-aromatic ring having 0-2 heteroatoms selected from oxygen nitrogen and sulfur; or a C₁-C₆aliphatic chain wherein up to two methylene units of the aliphatic chain are optionally replaced with —O—, —NR—, —C(O)—, or —S(O)ₙ; each L′ is optionally substituted with C₁-C₄aliphatic; —CN; halo; —OH; or a 3-6 membered non-aromatic ring having 0-2 heteroatoms selected from oxygen, nitrogen, and sulfur;
L² is H; a 3-7 membered aromatic or non-aromatic ring having 0-2 heteroatoms selected from oxygen nitrogen and sulfur; or a C₁-C₆aliphatic chain wherein up to two methylene units of the aliphatic chain are optionally replaced with —O—, —NR—, —C(O)—, or —S(O)ₙ; each L² is optionally substituted with C₁-C₄aliphatic; —CN; halo; —OH; or a 3-6 membered non-aromatic ring having 0-2 heteroatoms selected from oxygen, nitrogen, and sulfur; or
L¹ and L², together with the nitrogen to which they are attached, form a Ring D; Ring D is optionally substituted with 0-5 occurrences of J^{G};
Ring D is independently selected from a 3-7 membered heterocyclyl ring having 1-2 heteroatoms selected from oxygen, nitrogen and sulfur; or an 7-12 membered fully saturated or partially unsaturated bicyclic ring having 1-5 heteroatoms selected from oxygen, nitrogen, and sulfur;
J^{G} is independently selected from halo; —N(R^{o})₂; a 3-6 membered carbocycyl; a 3-6 membered heterocyclyl having 1-2 heteroatoms selected from oxygen nitrogen, or sulfur; or a C₁-C₄alkyl chain wherein up to two methylene units of the alkyl chain are optionally replaced with —O—, —NR—, —C(O)—, or —S(O)ₙ; each J^{G} is optionally substituted with 0-2 occurrences of J^{K}; or
   two occurrences of J^{G} on the same atom, together with the atom to which they are joined, form a 3-6 membered ring having 0-2 heteroatoms selected from oxygen, nitrogen, and sulfur; or two occurrences of J^{G}, together with Ring D, form a 6-10 membered saturated or partially unsaturated bridged ring system;
J^{K} is a 3-7 membered aromatic or non-aromatic ring having 0-2 heteroatoms selected from oxygen, nitrogen, and sulfur;
n is 0, 1, or 2; and
R and R^{o} are H or C₁-C₄alkyl.

In some embodiments, the ATR inhibitor is a compound of structural Formula I-B:

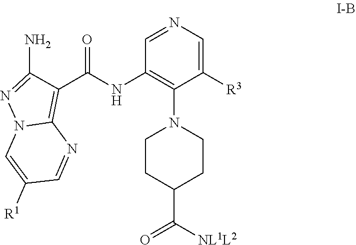

or a pharmaceutically acceptable salt thereof, wherein:
R¹ is independently selected from fluoro, chloro, and —C(J¹)₂CN;
J¹ is independently selected from H and C₁-C₂alkyl; or
   two occurrences of J′, together with the carbon atom to which they are attached, form an optionally substituted 3-4 membered carbocyclic ring;
R³ is independently selected from H; chloro; fluoro; C₁-C₄alkyl optionally substituted with 1-3 occurrences of halo; C₃-C₄cycloalkyl; and —CN;
L¹ is an optionally substituted C₁-C₆aliphatic;
L² is an optionally substituted C₁-C₆aliphatic; or
L¹ and L², together with the nitrogen to which they are attached, form a Ring D; Ring D is optionally substituted with 0-5 occurrences of J^{G};
Ring D is independently selected from a 3-7 membered heterocyclyl ring having 1-2 heteroatoms selected from oxygen, nitrogen and sulfur; and an 8-12 membered fully saturated or partially unsaturated bicyclic ring having 0-5 heteroatoms selected from oxygen, nitrogen, and sulfur;
J^{G} is independently selected from C₁-C₄alkyl, —N(R^{o})₂, and a 3-5 membered carbocycyl; or two occurrences of J^{G}, together with Ring D, form a 6-10 membered saturated or partially unsaturated bridged ring system; and
R^{o} is H or C₁-C₄alkyl.

In some embodiments, R¹ of formula I-B is fluoro. In some embodiments, R¹ of formula I-B is —CH₂CN. In some embodiments, R¹ of formula I-B is chloro.

In some embodiments, R³ of formula I-B is independently selected from H, chloro, fluoro, cyclopropyl, and C₁-C₄alkyl. In some embodiments, R³ of formula I-B is independently selected from H, chloro, and fluoro. In some embodiments, R³ of formula I-B is H. In some embodiments, R³ of formula I-B is chloro. In some embodiments, R³ of formula I-B is fluoro.

In some embodiments, the compound is represented by structural formula I-B, wherein L¹ and L² are independently selected from H; —(C₁-C₃alkyl)O(C₁-C₂alkyl); —(C₁-C₃alkyl)N(C₁-C₂alkyl)₂; C₁-C₄alkyl; azetidinyl; piperidinyl; oxytanyl; and pyrrolidinyl. In some embodiments, the compound is represented by structural formula I-B, wherein L¹ and L² are C₁-C₃alkyl.

In some embodiments, the compound is represented by structural formula I-B, wherein L¹ and L², together with the nitrogen to which they are attached, form Ring D.

In some embodiments, the compound is represented by structural formula I-B, wherein Ring D is a 3-7 membered heterocyclyl ring having 1-2 heteroatoms selected from oxygen, nitrogen, and sulfur. In some embodiments, the compound is represented by structural formula I-B, wherein Ring D is independently selected from piperazinyl, piperidinyl, morpholinyl, tetrahydopyranyl, azetidinyl, pyrrolidinyl, and 1,4-diazepanyl. In some embodiments, the compound is represented by structural formula I-B, wherein Ring D is piperazinyl, piperidinyl, 1,4-diazepanyl, pyrrolidinyl and azetidinyl. In some embodiments, the compound is represented by structural formula I-B, wherein Ring D is piperidinyl or piperazinyl. In some embodiments, Ring D is piperazinyl.

In some embodiments, the compound is represented by structural formula I-B, wherein Ring D is an 8-12 membered fully saturated or partially unsaturated bicyclic ring having 0-5 heteroatoms selected from oxygen, nitrogen, and sulfur. In some embodiments, the compound is represented by structural formula I-B, wherein Ring D is octahydropyrrolo[1,2-a]pyrazine or octahydropyrrolo[3,4-c]pyrrole. In some embodiments, Ring D is octahydropyrrolo[1,2-a]pyrazine.

In some embodiments, the compound is represented by structural formula I-B, wherein J^{G} is halo, C₁-C₄alkyl, —O(C₁-C₃alkyl), C₃-C₆cycloalkyl, a 3-6 membered heterocyclyl, —NH(C₁-C₃alkyl), —OH, or —N(C₁-C₄alkyl)₂. In some embodiments, the compound is represented by structural formula I-B, wherein J^{G} is methyl, —N(C₁-C₄alkyl)₂, ethyl, —O(C₁-C₃alkyl), cyclopropyl, oxetanyl, cyclobutyl, pyrrolidinyl, piperidinyl, or azetidinyl. In some embodiments, the compound is represented by structural formula I-B, wherein J^{G} is methyl, —O(C₁-C₃alkyl), oxetanyl, pyrrolidinyl, piperidinyl, or azetidinyl. In some embodiments, the compound is represented by structural formula I-B, wherein J^{G} is C₁-C₄alkyl, C₃-C₅cycloalkyl, or —N(C₁-C₄alkyl)₂. In some embodiments, the compound is represented by structural formula I-B, wherein J^{G} is methyl, ethyl, or cyclopropyl. In some embodiments, the compound is represented by structural formula I-B, wherein J^{G} is methyl. In some embodiments, the compound is represented by structural formula I-B, wherein J^{G} is oxetanyl.

In some embodiments, the compound is represented by structural formula I-B, wherein two occurrences of J^{G}, together with Ring D, form a 6-10 membered saturated or partially unsaturated bridged ring system. In some embodiments, the compound is represented by structural formula I-B, wherein the bridged ring system is 1,4-diazabicyclo[3.2.2]nonane, 1,4-diazabicyclo[3.2.1]octane, or 2,5-diazabicyclo [2.2.1]heptane. In some embodiments, the compound is represented by structural formula I-B, wherein the bridged ring system is 1,4-diazabicyclo[3.2.2]nonane.

In some embodiments, the compound is represented by structural formula I-B, wherein two occurrences of J^{G} on the same atom, together with the atom to which they are joined, form a 3-6 membered ring having 0-2 heteroatoms selected from oxygen, nitrogen, and sulfur. In some embodiments, the compound represented by structural formula I-B, wherein the ring formed by the two occurrences of J^{G} on the same atom is oxetanyl or cyclopropyl.

In some embodiments, the ATR inhibitor is a compound of structural formula I, I-A, and I-B, wherein the compounds are represented in Table 3.

TABLE 3

| | |
|---|---|
| 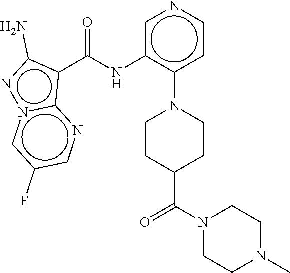 | I-G-1 |
| 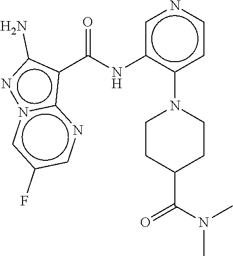 | I-G-2 |
| 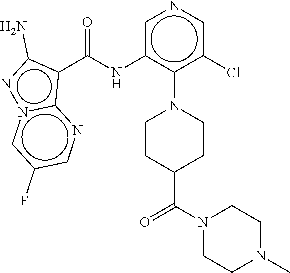 | I-G-3 |
| 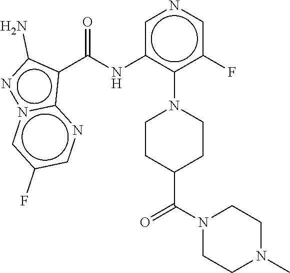 | I-G-4 |
| 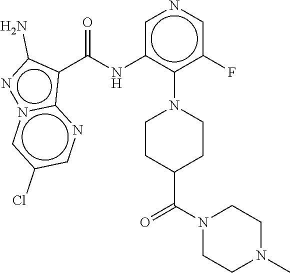 | I-G-5 |
| 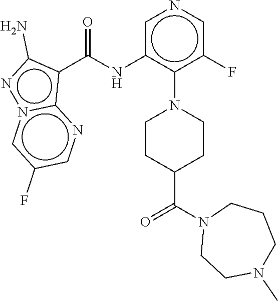 | I-G-6 |
| 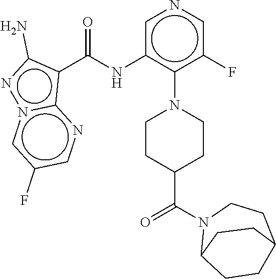 | I-G-7 |
| 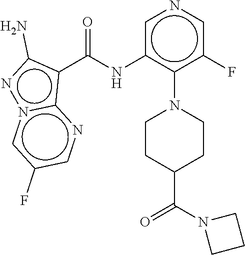 | I-G-8 |
| 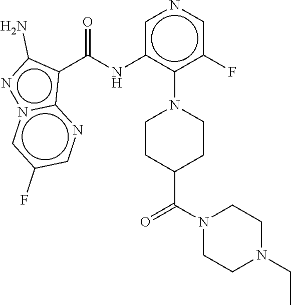 | I-G-9 |
| 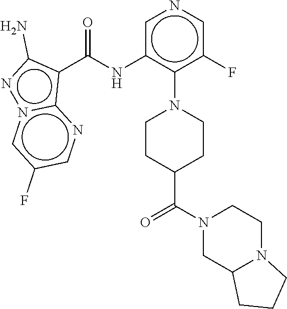 | I-G-10 |
| 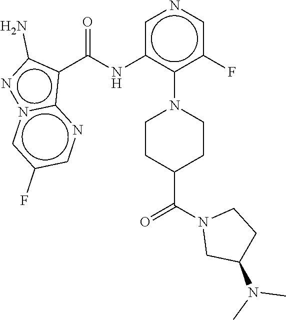 | I-G-11 |
| 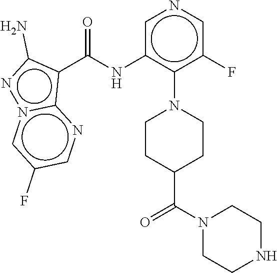 | I-G-12 |
| 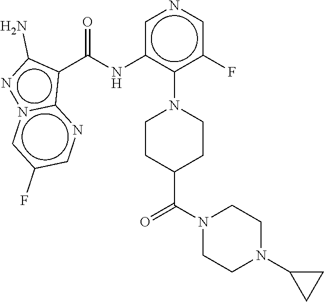 | I-G-13 |
| 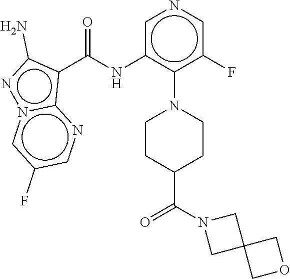 | I-G-14 |
| 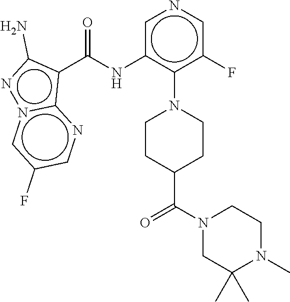 | I-G-15 |
| 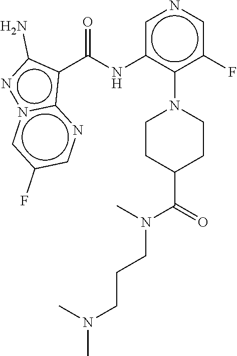 | I-G-16 |
| 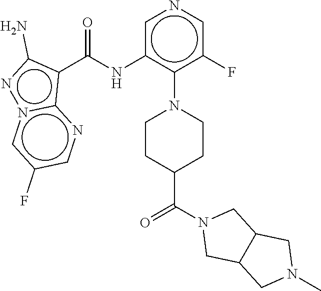 | I-G-18 |
| 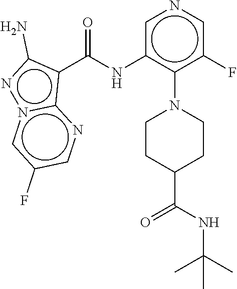 | I-G-19 |
| 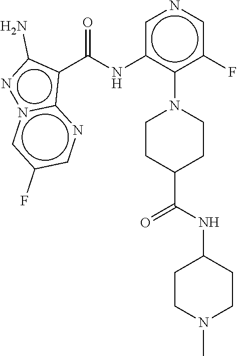 | I-G-20 |
| 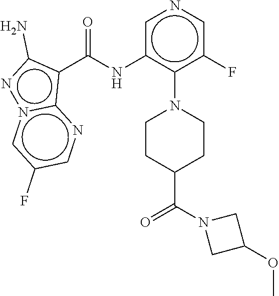 | I-G-21 |
| 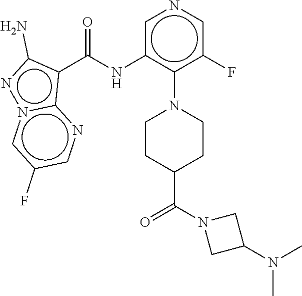 | I-G-22 |
| 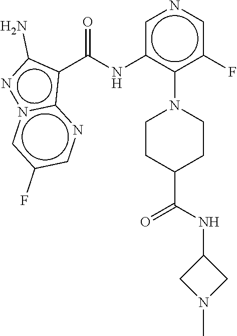 | I-G-23 |
| 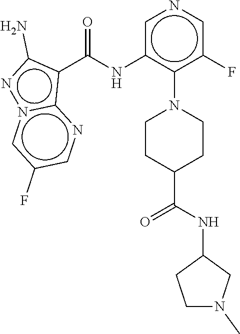 | I-G-24 |
| 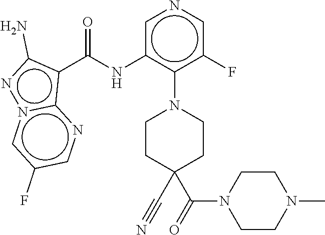 | I-G-25 |
| 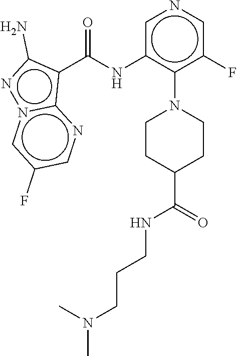 | I-G-26 |
| 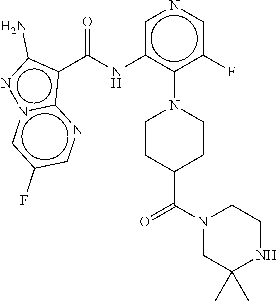 | I-G-27 |
| 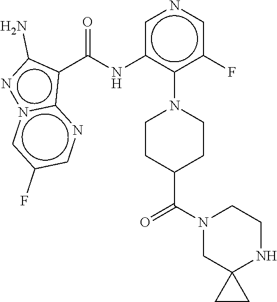 | I-G-28 |
| 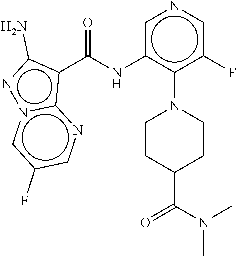 | I-G-29 |
| 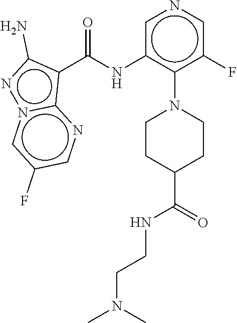 | I-G-30 |
| 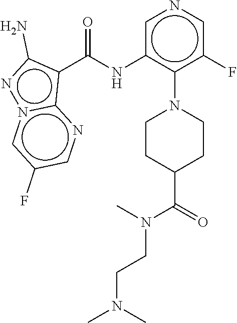 | I-G-31 |
| 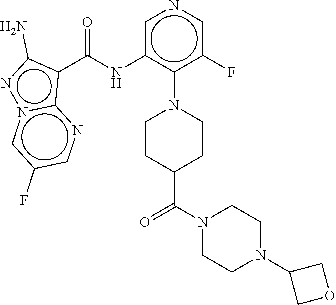 | I-G-32 |
| 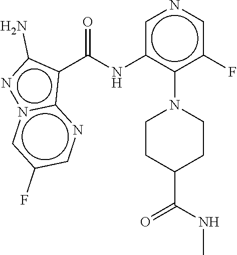 | I-G-33 |
| 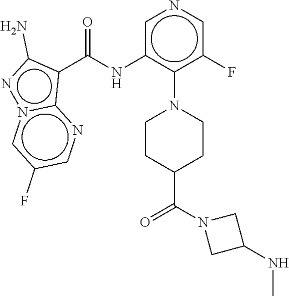 | I-G-34 |
| 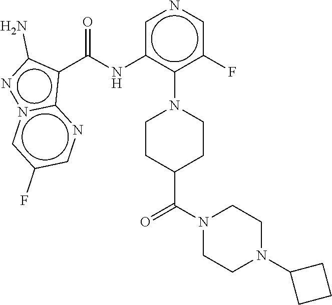 | I-G-35 |

or a pharmaceutically acceptable salt thereof.

In some embodiments, the ATR inhibitor is:

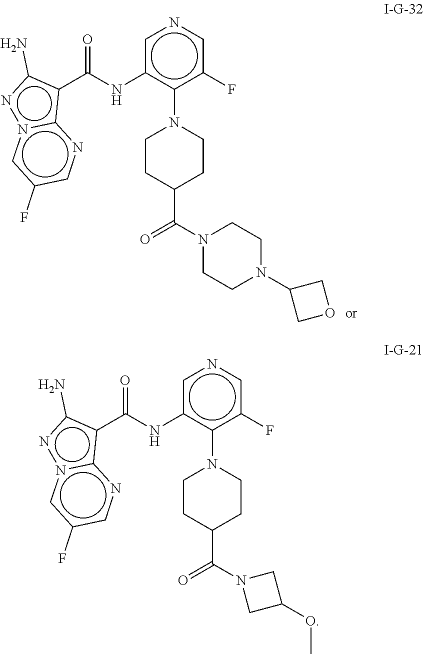

or a pharmaceutically acceptable salt thereof.

In some embodiments, the ATR inhibitor is:

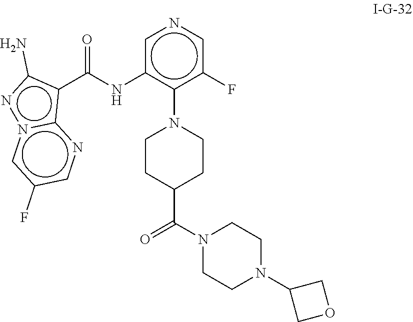

or a pharmaceutically acceptable salt thereof.

Second Therapeutic Agents and Combination Therapy

In some embodiments, the method of identifying, selection and/or treatment of a cancer is based on the sensitivity of the cancer for an ATR inhibitor. In some embodiments, the method of identifying, selection and/or treatment of a cancer is based on the sensitivity of the cancer for the ATR inhibitor in combination with a second therapeutic agent, particularly an anticancer agent, more particularly where the second therapeutic agent is a DNA damaging agent. In some embodiments, the ATR inhibitor is used in combination with one or more DNA damaging agents. In some embodiments, the second therapeutic agent is a DNA damage enhancing agent, such as PARP inhibitor or Chk1 inhibitor. In some embodiments, the ATR inhibitor is used in combination with one or more DNA damaging agents, and one or more DNA damage enhancing agents, e.g., PARP inhibitor, Chk1 inhibitor, or combinations thereof.

In some embodiments, the method of identifying, selection and/or treatment of a cancer is for the ATR inhibitor in combination with a DNA-damaging agent. In some embodiments, the DNA-damaging agent includes, by way of example and not limitation, a platinating agent, topoisomerase I (Topo I) inhibitor, topoisomerase II (Topo II) inhibitor, anti-metabolite (e.g., purine antagonists and pyrimidine antagonists), alkylating agents, and anti-cancer antibiotic. In some embodiments, the ATR inhibitor is used in combination with ionizing radiation.

In some embodiments, the DNA damaging agent is a platinating agent. In some embodiments, the platinating agent is, for example, cisplatin, oxaliplatin, carboplatin, nedaplatin, satraplatin and other derivatives, such as lobaplatin, triplatin, tetranitrate, picoplatin, ProLindac or Aroplatin.

In some embodiments, the DNA damaging agent is a Topo I inhibitor. In some embodiments, the Topo I inhibitor is, for example, camptothecin, topotecan, irinotecan, rubitecan, or belotecan.

In some embodiments, the DNA damaging agent is a Topo II inhibitor. In some embodiments, the Topo II inhibitor is, for example, etoposide, daunorubicin, doxorubicin, mitoxantrone, aclarubicin, epirubicin, idarubicin, amrubicin, amsacrine, pirarubicin, valrubicin, zorubicin or teniposide.

In some embodiments, the DNA damaging agent is an antimetabolite. In some embodiments, the anti-metabolite is, for example, hydroxyurea, methotrexate, pemetrexed thioguanine, fludarabine, cladribine, 6 mercaptopurine, cytarabine, gemcitabine, or 5-fluorouracil (5FU).

In some embodiments, the DNA damaging agent is an alkylating agent. In some embodiments, the alkylating agent includes, by way of example and not limitation, nitrogen mustards, nitrosoureas, triazenes, alkyl sulphonates, procarbazine and aziridines. In some embodiments, the alkylating agent is, for example, cyclophosphamide, ifosfamide, trofosfamide, chlorambucil, melphalan, prednimustine, bendamustine, uramustine, estramustine, carmustine, lomustine, semustine, fotemustine, nimustine, ranimustine, streptozocin, busulfan, mannosulfan, treosulfan, carboquone, triaziquone, mechlorethamine, triethylenemelamine, procarbazine, dacarbazine, mitozolomide, or temozolomide.

In some embodiments, the DNA damaging agent is an anti-cancer antibiotic. In some embodiments, the anti-cancer antibiotic is, for example, mitoxantrone, bleomycin, mitomycin C, or actinomycin.

In some embodiments, the DNA damaging agent is cisplatin, oxaliplatin, carboplatin, nedaplatin, satraplatin, lobaplatin, triplatin, tetranitrate, picoplatin, ProLindac, Aroplatin, camptothecin, topotecan, irinotecan, rubitecan, belotecan, etoposide, daunorubicin, doxorubicin, mitoxantrone, aclarubicin, epirubicin, idarubicin, amrubicin, amsacrine, pirarubicin, valrubicin, zorubicin, teniposide, hydroxyurea, methotrexate, pemetrexed thioguanine, fludarabine, cladribine, 6 mercaptopurine, cytarabine, gemcitabine, 5-fluorouracil (5FU), cyclophosphamide, ifosfamide, trofosfamide, chlorambucil, melphalan, prednimustine, bendamustine, uramustine, estramustine, carmustine, lomustine, semustine, fotemustine, nimustine, ranimustine, streptozocin, busulfan, mannosulfan, treosulfan, carboquone, triaziquone, mechlorethamine, triethylenemelamine, procarbazine, dacarbazine, mitozolomide, temozolomide, mitoxantrone, bleomycin, mitomycin C, or actinomycin. In some embodiments, one or more of the DNA damaging agents can be used, concurrently or sequentially.

In some embodiments, the second therapeutic agent is a DNA damage enhancing agent. In some embodiments, the DNA damage enhancing agent is a poly ADP ribose polymerase (PARP) inhibitor. In some embodiments, the PARP inhibitor is an inhibitor of PARP1, PARP2, PARP3, or combinations thereof. In some embodiments, the PARP inhibitor is, for example, olaparib (AZD2281 or KU-0059436), veliparib (ABT-888), rucaparib (PF-01367338), CEP-9722, INO 1001, niraparib (MK-4827), E7016, talazoparib (BMN673), AZD2461, or combinations thereof.

In some embodiments, the DNA damage enhancing agent is a Chk1 inhibitor. In some embodiments Chk1 inhibitor is, for example, AZD7762, LY2603618, MK-8776, CHIR-124, CCT245737, PF-477736, or combinations thereof.

In some embodiments, the method of identifying, selection and/or treatment of a cancer is with a combination therapy comprising an ATR inhibitor of formula (IIA-7):

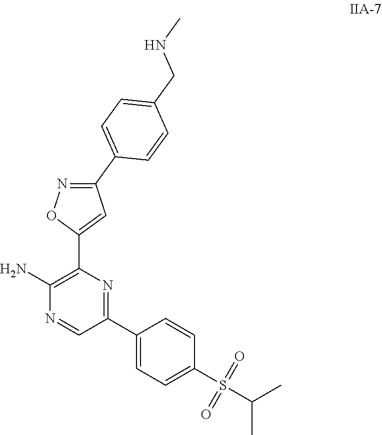 [SMILES: CNCc1ccc(-c2cc(-c3nc(-c4ccc(S(=O)(=O)C(C)C)cc4)cnc3N)on2)cc1]

or a pharmaceutically acceptable salt thereof, and cisplatin.

In some embodiments, the identifying, selection, and/or treatment of a cancer is for a combination therapy comprising the ATR inhibitor IIA-7 above, or a pharmaceutically acceptable salt thereof, and gemcitabine.

In some embodiments, the identifying, selection, and/or treatment of a cancer is for a combination therapy comprising an ATR inhibitor for formula (I-G-32):

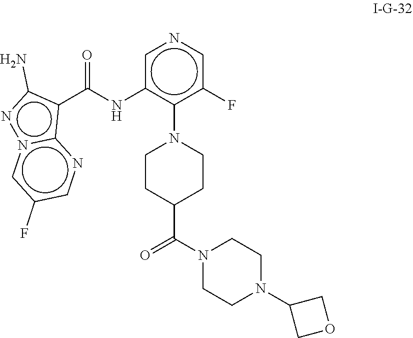

or a pharmaceutically acceptable salt thereof, and cisplatin.

In some embodiments, the identifying, selection, and/or treatment of a cancer is for a combination therapy comprising the ATR inhibitor I-G-32 above, or a pharmaceutically acceptable salt thereof, and gemcitabine.

In some embodiments, one or more other additional cancer therapy can be used together with the foregoing combination of the ATR inhibitor and second therapeutic agent, or in some embodiments, the method of identifying, selection, and/or treatment of a cancer can be for an ATR inhibitor in combination with one or more the other additional cancer therapy, such as radiation therapy, chemotherapy, or other standard agents used in cancer therapy, for example radiosensitizers, chemosensitizers, and DNA repair modulators (e.g., PARP and Chk1 inhibitors). Radiosensitizers are agents that can be used in combination with radiation therapy, where the radiosensitizer acts, among others, to making cancer cells more sensitive to radiation therapy, working in synergy with radiation therapy to provide an improved synergistic effect, acting additively with radiation therapy, or protecting surrounding healthy cells from damage caused by radiation therapy. Chemosensitizers are agents that can be used in combination with chemotherapy. where the chemosensitizers acts, among others, to making cancer cells more sensitive to chemotherapy, working in synergy with chemotherapy to provide an improved synergistic effect, acting additively to chemotherapy, or protecting surrounding healthy cells from damage caused by chemotherapy.

In some embodiments, the additional cancer therapy can include, for example, immunotherapy, for example, antibody therapy or cytokine therapy or other immunomodulator therapy, such as interferons, interleukins, and tumor necrosis factor (TNF). Any combination of these cancer therapies may be used together with the combination therapy described herein.

In some embodiments, the second therapeutic agent or other additional cancer therapy can be an chemotherapeutic drugs, including, but not limited to, spindle poisons (e.g., vinblastine, vincristine, vinorelbine, paclitaxel, etc.), podophyllotoxins (e.g., etoposide, irinotecan, topotecan), nitrosoureas (e.g., carmustine, lomustine), inorganic ions (e.g., cisplatin, carboplatin), enzymes (asparaginase), and hormones (e.g., tamoxifen, leuprolide, flutamide, and megestrol), Gleevec™, adriamycin, dexamethasone, and cyclophosphamide.

In some embodiments, the second therapeutic agent or other additional cancer therapy can include, among others, abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar R); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (RituxanC); sargramostim (Leukine®); Sargramostim (Prokine®); sorafenib (Nexavar R); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex R); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); zoledronate (Zometa®) and vorinostat (Zolinza®).

Pharmaceutical Compositions

In some embodiments, the ATR inhibitors and other therapeutic agents (e.g., DNA-damaging agents) or pharmaceutical salts thereof can be formulated separately or together into pharmaceutical compositions for administration. In various embodiments, each therapeutic agent can be formulated in a pharmaceutical composition that comprises the agent and a pharmaceutically acceptable carrier. Suitable pharmaceutical carriers are described herein and in Remington: The Science and Practice of Pharmacy, 21st Ed. (2005). The therapeutic compounds and their physiologically acceptable salts can be formulated for administration by any suitable route, including, among others, topically, nasally, orally, parenterally, rectally or by inhalation. In some embodiments, the administration of the pharmaceutical composition can be prepared for intradermal, subdermal, intravenous, intramuscular, intranasal, intracerebral, intratracheal, intraarterial, intraperitoneal, intravesical, intrapleural, intracoronary or intratumoral administration, such as for injection with a syringe or other devices. Transdermal administration is also contemplated, as are inhalation or aerosol administration. Tablets, capsules, and solutions can be administered orally, rectally or vaginally.

For oral administration, a pharmaceutical composition can take the form of, for example, a tablet or a capsule prepared by conventional means with a pharmaceutically acceptable excipient. Tablets and capsules comprising the active ingredient can be prepared together with excipients such as: (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; (c) binders, e.g., magnesium aluminum silicate, starch paste, gelatin, tragacanth, methylcellulose, sodium carboxymethylcellulose, polyvinylpyrrolidone and/or hydroxypropyl methylcellulose; (d) disintegrants, e.g., starches (including potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners. The compositions are prepared according to conventional mixing, granulating or coating methods. Tablets may be either film coated or enteric coated according to methods known in the art.

Liquid preparations for oral administration can take the form of, for example, solutions, syrups, or suspensions, or they can be presented as a dry product for reconstitution with water or other suitable vehicle before use. Such liquid preparations can be prepared by conventional means with pharmaceutically acceptable carriers and additives, for example, suspending agents, e.g., sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

The therapeutic agents can be formulated for parenteral administration, for example by bolus injection or continuous infusion. Formulations for injection can be presented in unit dosage form, for example, in ampoules or in multi-dose containers, with an optionally added preservative. Injectable compositions can be aqueous isotonic solutions or suspensions. In some embodiments for parenteral administration, the therapeutic agents can be prepared with a surfactant, or lipophilic solvents, such as triglycerides or liposomes. The compositions may be sterilized and/or contain adjuvants, such as preserving, stabilizing, wetting or emulsifying agents, solution promoters, salts for regulating the osmotic pressure and/or buffers. Alternatively, the therapeutic agent can be in powder form for reconstitution with a suitable vehicle, for example, sterile pyrogen-free water, before use. In addition, they may also contain other therapeutically effective substances.

For administration by inhalation, the therapeutic agent may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a therapeutic agent with a carrier. Preferred carriers include absorbable pharmacologically acceptable solvents to assist passage through the skin of the subject. For example, transdermal devices are in the form of a bandage or patch comprising a backing member, a reservoir containing the therapeutic agent optionally with carriers, optionally a rate controlling barrier to deliver the compound to the skin of the host at a controlled and predetermined rate over a prolonged period of time, and a means to secure the device to the skin. Matrix transdermal formulations may also be used.

Suitable formulations for topical application, e.g., to the skin and eyes, are preferably aqueous solutions, ointments, creams or gels known in the art. The formulations may contain solubilizers, stabilizers, tonicity enhancing agents, buffers and preservatives.

In some embodiments, the therapeutic agent can also be formulated as a rectal composition, for example, suppositories or retention enemas, for example, containing conventional suppository bases, for example, cocoa butter or other glycerides, or gel forming agents, such as carbomers.

In some embodiments, the therapeutic agent can be formulated as a depot preparation. Such long-acting formulations can be administered by injection or implantation (for example, subcutaneously or intramuscularly). The therapeutic agent can be formulated with suitable polymeric or hydrophobic materials (for example as an emulsion in an acceptable oil), ion exchange resins, biodegradable polymers, or as sparingly soluble derivatives, for example, as a sparingly soluble salt.

The pharmaceutical compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

Administration and Dosages

In some embodiments, a pharmaceutical composition of the therapeutic agent is administered to a subject, preferably a human, at a therapeutically effective amount or a therapeutically effective dose to prevent, treat, or control a condition or disease as described herein. As used herein, “treating” or “treatment” of a disease, disorder, or syndrome, as used herein, includes (i) preventing the disease, disorder, or syndrome from occurring in a subject, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome.

The specific effective dose level for any particular patient will depend upon a variety of factors including the type and stage of cancer being treated; the activity of the specific agent; the specific composition employed; the age, body weight, general health, sex and diet of the patient; the time of administration, route of administration, and rate of excretion of the specific agent employed; the duration of the treatment; drugs used in combination or coincidental with the specific compound employed, and like factors well known in the medical arts.

The pharmaceutical composition is administered to a subject in an amount sufficient to elicit an effective therapeutic response in the subject. An effective therapeutic response is a response that at least partially arrests or slows the symptoms or complications of the condition or disease. An amount adequate to accomplish this is defined as “therapeutically effective dose” or “therapeutically effective amount.” The expression “dosage unit form” as used herein refers to a physically discrete unit of agent appropriate for the patient to be treated. It will be understood, however, that the total daily usage of the agents and compositions of the present invention will be decided by the attending physician within the scope of sound medical judgment.

In some embodiments, the amount of the therapeutic agent can be an amount that is less than the effective amount when the agent is used alone but that is effective to treat one or more of the cancers recited herein when used in combination with another agent, e.g., a second therapeutic agent. Thus, in some embodiments, the combination therapy is referred to be as being administered in an therapeutically effective amount, including for example a therapeutically effective amount that results in a synergistic response (e.g., a synergistic anti-cancer response).

In some embodiments, a suitable dosage of the therapeutic agent, e.g., ATR inhibitor, or a composition thereof is from about can be administered orally or parenterally at dosage levels of about 0.01 to about 100 mg/kg, about 0.01 mg/kg to about 50 mg/kg and preferably from about 1 mg/kg to about 25 mg/kg, of subject body weight per day to obtain the desired therapeutic effect. In some embodiments, the dose of the compound can be administered once per day or divided into subdoses and administered in multiple doses, e.g., twice, three times, or four times per day to obtain the desired therapeutic effect.

As discussed above, the ATR inhibitor compound can be administered with one or more of a second therapeutic agent, separately, sequentially or concurrently, either by the same route or by different routes of administration. When administered sequentially, the time between administrations is selected to benefit, among others, the therapeutic efficacy and/or safety of the combination treatment. In some embodiments, the ATR inhibitor can be administered first followed by a second therapeutic agent, or alternatively, the second therapeutic agent administered first followed by the ATR inhibitor. For example, the ATR inhibitor can be administered followed by administration of a therapeutically effective amount of the second therapeutic agent, where the second therapeutic agent is administered within about 48, 36, 24, 12, 6, 4 or 2 hours after the administration of the ATR inhibitor. In some embodiments, the ATR inhibitor is administered after administration of the second therapeutic agent (e.g., the DNA-damaging agent). For example, a therapeutically effective amount of the second therapeutic agent is administered followed by administration of the ATR inhibitor, where the ATR inhibitor is administered within about 48, 36, 24, 12, 6, 4 or 2 hours of the administration of the second therapeutic agent. In some embodiments, the ATR inhibitor and the second therapeutic agent is administered repeatedly on a predetermined schedule, including for example daily, every 2 days, every 3 days, every 4 days, every 5 days, every 6 days, every 7 days (every week), every 8 days, every 9 days, every 10 days, every 11 days, every 12 days, every 13 days, every 14 days (every two weeks), every month, etc. The frequency of administration of the ATR inhibitor may be different from the second therapeutic agent.

When administered concurrently, the ATR inhibitor compound can be administered separately at the same time as the second therapeutic agent, by the same or different routes, or administered in a single composition by the same route. In some embodiments, the amount and frequency of administration of the second therapeutic agent can use standard dosages and standard administration frequencies used for the particular therapeutic agent. See, e.g., Physicians' Desk Reference, 70th Ed., PDR Network, 2015; incorporated herein by reference.

In some embodiments where the ATR inhibitor is administered in combination with a second therapeutic agent, the dose of the second therapeutic agent is administered at a therapeutically effective dose. In some embodiments, guidance for dosages of the second therapeutic agent is provided in Physicians' Desk Reference, 70^{th} Ed, PDR Network (2015), incorporated herein by reference. In some embodiments, a suitable dose, depending on the second therapeutic agent, can be from about 1 ng/kg to about 1000 mg/kg, from about 0.01 mg/kg to about 900 mg/kg, from about 0.1 mg/kg to about 800 mg/kg, from about 1 mg/kg to about 700 mg/kg, from about 2 mg/kg to about 500 mg/kg, from about 3 mg/kg to about 400 mg/kg, from about 4 mg/kg to about 300 mg/kg, or from about 5 mg/kg to about 200 mg/kg. In some embodiments, the dose of the second therapeutic agent can be administered once per day or divided into subdoses and administered in multiple doses, e.g., twice, three times, or four times per day.

The following examples are provided to further illustrate the methods of the present disclosure, and the compounds and compositions for use in the methods. The examples described are illustrative only and are not intended to limit the scope of the invention in any way.

### EXAMPLES

### Example 1. Identification of Predictive Biomarkers to ATR Inhibitor IIA-7 or I-G-32 in Combination with Cisplatin or Gemcitabine

The primary objective of this study was to assess the in vitro response of a panel of 552 cancer cell lines to ATR inhibitor compounds IIA-7 and I-G-32, in combination with the cytotoxic agent cisplatin or gemcitabine.

In addition to evaluating cellular response to the combination treatments, the secondary objective of this study was to perform a preliminary assessment of the relationship between baseline biomarkers (e.g., mutations in the tumor protein 53 (TP53) or baseline gene expression) and response to various combinations of the therapeutic agents.

This study was performed at Horizon Discovery using 552 cancer cell lines, which included lines derived from lung cancer, colorectal cancer, ovarian cancer, skin cancer, B cell lymphoma, breast cancer, and other cancers.

The results indicated that ATR inhibitor compounds IIA-7 and I-G-32 are synergistic when combined with cisplatin and gemcitabine. In agreement with previous in vitro studies, TP53 mutation was associated with response to both compounds IIA-7 and I-G-32 in combination with cisplatin or gemcitabine. Additionally, baseline CDKN1A gene expression was found to be associated with ATR inhibitor synergy in a 251 cell line subset of the screen. The association was validated in a non-overlapping 182 cell line subset of the screen. This study was not required to be conducted in accordance with US Food and Drug Administration Good Laboratory Practice Regulations (21 CFR 58).

The objectives of this study were to assess cell sensitivity to ATRi in combination with cytotoxic agents (cisplatin and gemcitabine), assess the association between TP53 mutation status and ATRi synergy, and to identify candidate baseline gene expression biomarkers that broadly associate with ATRi synergy.

Cell Culture Methods. Cells were removed from liquid nitrogen storage, thawed and expanded in appropriate growth media. Once expanded, cells were seeded in 384-well tissue culture treated plates at 500 cells per well. After 24 hours, cells were treated for either 0 hours or treated for 96 hours with compound IIA-7 or I-G-32 in combination with the DNA-damaging agents listed in Table 4. At the end of either 0 hours or 96 hours, cell status was analyzed using ATPLite (adenosine triphosphate monitoring system; Perkin Elmer) to assess the biological response of cells to drug combinations.

TABLE 4
Listing of reagents

| | Starting | | | | |
|---|---|---|---|---|---|
| | Concentration | | | | |
| Vertex | of Vertex | | | | |
| Compound | Compound | SOC | Vendor | Catalog # | SOC MoA |
| IIA-7 and | 50 nM and 250 nM | Cisplatin | Enzo | ALX-400- | DNA crosslinker |
| I-G-32 | (IIA-7); | | | 040-M050 | |
| | 10 nM and 50 nM | Gemcitabine | Sigma | G6424 | Nucleoside analog |
| | (I-G-32) | | | | |

In this study, growth inhibition (GI) was used as the primary endpoint. ATP monitoring was performed using ATPLite, which allows for the monitoring of cytocidal, cytostatic and proliferative effects of drugs on cells. A summary of the cell line types represented in the screen is listed in Table 5.

TABLE 5
Summary of cell line types in screen

| Tumor Type | Number of cell lines |
|---|---|
| acute myeloid leukemia | 12 |
| B cell lymphoma | 39 |
| bile duct | 7 |
| bladder | 5 |
| bone | 7 |
| breast | 35 |
| chronic myeloid leukemia | 1 |
| colorectal | 48 |
| endometrium | 28 |
| esophageal | 23 |
| gastric | 32 |
| glioma | 12 |
| head/neck | 29 |
| kidney | 8 |
| liver | 25 |
| medulloblastoma | 2 |
| mesothelioma | 8 |
| multiple myeloma | 19 |
| neuroblastoma | 10 |
| Non-small cell lung cancer | 55 |
| ovary | 43 |
| pancreas | 26 |
| prostate | 3 |
| small cell lung cancer | 18 |
| skin | 41 |
| soft tissue | 5 |
| T cell lymphoma | 9 |
| thyroid | 2 |

Data Analysis to Assess Synergy of Combination Treatments. Data analysis was performed using R programming (R Core Team (2014). R: A language and environment for statistical computing. R Foundation for Statistical Computing, Vienna, Austria.). Synergy was evaluated using the sum of the AUC (Area Under the Curve) difference.

Briefly, combination treatment effect was calculated as the AUC normalized to the single agent effect of the ATR inhibitor compound. Total synergy or antagonism was calculated as the difference between the normalized combination AUC and the genotoxin single agent AUC. As before, the synergy score was normalized by dividing the total synergy score by the total number of regimens used.

Gene Mutational Status Determination. Mutation calls were obtained from Sanger's Cell Line Project exome sequencing project and the Broad Institute's CCLE hybrid capture and Raindance targeted cell line sequencing data. For 1506 genes sequenced in all three datasets, consensus mutation calls were obtained for 264 ORID cell lines. Cell lines were scored as mutant if there was at least one consensus nonsynonymous mutation, and wild type if there was no mutation call. Analysis was limited to 396 genes that had 10 or more mutation calls among the 264 cell lines.

Gene Expression and Data Processing. Pre-treatment gene expression values were determined by microarray on 502 of the cancer cell lines in the screen. RNA was isolated and the concentration and integrity were measured via bioanalysis and gel electrophoresis respectively. RNA samples were processed to generate labeled material for hybridization to the Affymetrix Prime View array. Hybridization, wash, and scanning on the Affymetrix system was per the Affymetrix protocol at HudsonAlpha. Arrays were background corrected and normalized and gene expression values were obtained using the RMA (Robust Multiarray Averaging) algorithm. Global gene expression was assessed using the Bioconductor package arrayQualityMetrics, and arrays that passed the assessment were retained for further analysis.

Association Analysis. Association of synergy between compound IIA-7 or compound I-G-32 in combination with cisplatin or gemcitabine (ATR inhibitor synergy) and baseline gene expression or gene mutational status was assessed using ANOVA. Covariates with significant association with ATRi synergy with a particular agent were retained in the ANOVA model. In cases where multiple potential biomarkers were assessed with respect to a single endpoint, multiple test correction was performed using the FDR procedure (see Benjamini Y and Hochberg Y., 1995, “Controlling the False Discovery Rate: A Practical and Powerful Approach to Multiple Testing,” J Royal Statistical Soc. Series B (Methodological), 57:289-300).

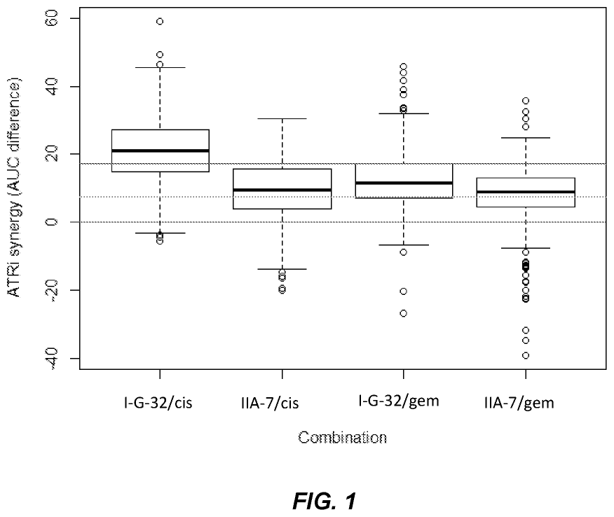

Results. Sensitivity of cancer cells to compound IIA-7 or compound I-G-32 in combination with cisplatin or gemcitabine was evaluated in 552 cancer cell lines. Synergy was seen in all combinations tested (see FIG. 1). This study also identified an association between TP53 mutational status and response to compound IIA-7 in combination with gemcitabine or cisplatin as well as compound I-G-32 in combination with gemcitabine or cisplatin.

An initial study also examined 264 cancer cell lines to determine whether any gene mutation was associated with response to the ATR inhibitor combination treatment. In this set of cell lines, the data indicated an association between TP53 mutational status and synergistic response to compound IIA-7 with gemcitabine (FDR q value: 0.047) and compound I-G-32 with gemcitabine (FDR q value: 0.035). No other gene out of the 396 tested was found to have significant association between mutational status and response to the ATR inhibitors with either gemcitabine or cisplatin. In addition, the association between TP53 mutational status and compound I-G-32/cisplatin synergy was stronger than for any of the other 396 genes tested (unadjusted p value: 0.0031, FDR q value not significant).

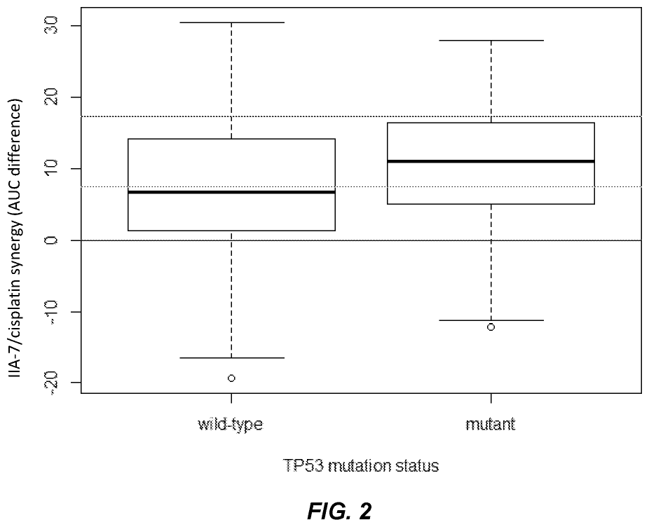

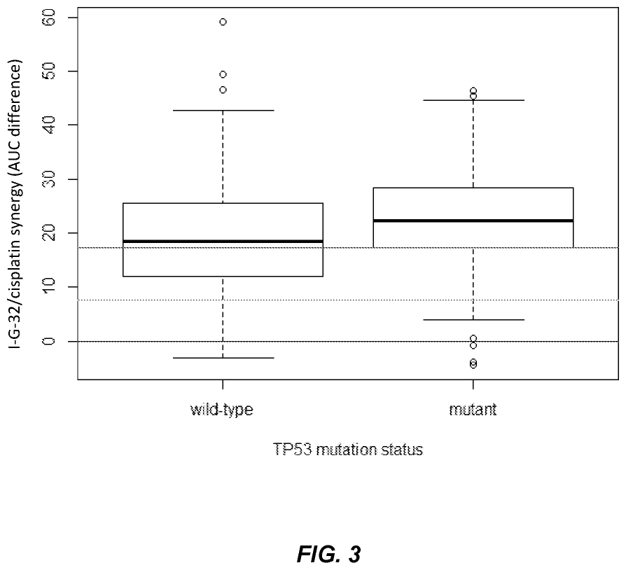

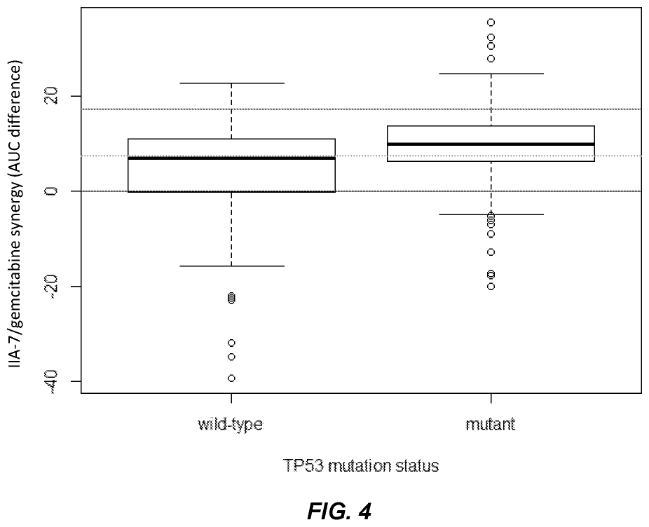

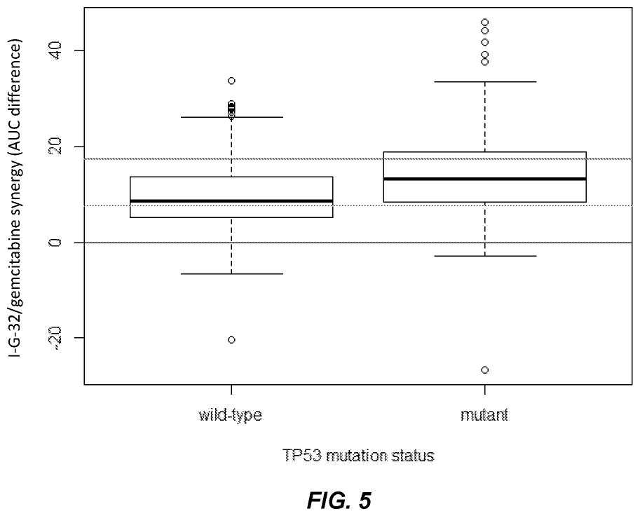

Given the association seen between TP53 mutational status and response in the panel of 264 cancer cell lines described above, the relationship between TP53 mutational status and ATR inhibitor synergy was evaluated as an a priori hypothesis using an expanded set of 552 cancer cell lines. A strong, statistically significant relationship was observed between TP53 mutational status and synergistic response to compound IIA-7 and compound I-G-32 in combination with the cytotoxic agents cisplatin or gemcitabine in this expanded panel of 552 cancer cell lines (ANOVA p value range: 2.6×10-7 to 4.5×10-3) (FIGS. 2, 3, 4 and 5). On the other hand, there was no significant association found between TP53 mutational status and single agent ATR inhibitor activity (data not shown).

To examine the association between gene expression and ATR inhibitor synergy, an initial study used a set of 251 cancer cell lines. The data from this set of cell lines showed an association between baseline CDKN1A gene expression and synergistic response for all combinations of ATR inhibitor and genotoxic agents cisplatin and gemcitabline, except for compound IIA-7 in combination with cisplatin (FDR range: 1.1×10-7 to 7.5×10-2). Because of the breadth of the association and the known role of CDKN1A as a downstream transcriptional target gene of TP53, CDKN1A was selected as a candidate biomarker, and examined on a non-overlapping set of 182 cancer cell lines, the results of which confirmed the association between baseline CDKN1A gene expression and synergistic response to the ATR inhibitor combination treatments (ANOVA p value range: 1.2×10-6 to 4.7×10-4).

As a test of the specificity of this candidate biomarker, 47 genes whose expression was associated with synergistic response (FDR q value<0.1) for at least three of the ATR inhibitor combinations in the initial set of 251 cancer cell lines were further evaluated in a 182 cell line validation set. Only CDKN1A had a transcriptome-wide significant association between baseline gene expression and synergistic response for more than one combination of ATR inhibitors and genotoxic agents (FDR q value<0.1 in three combinations for CDKN1A).

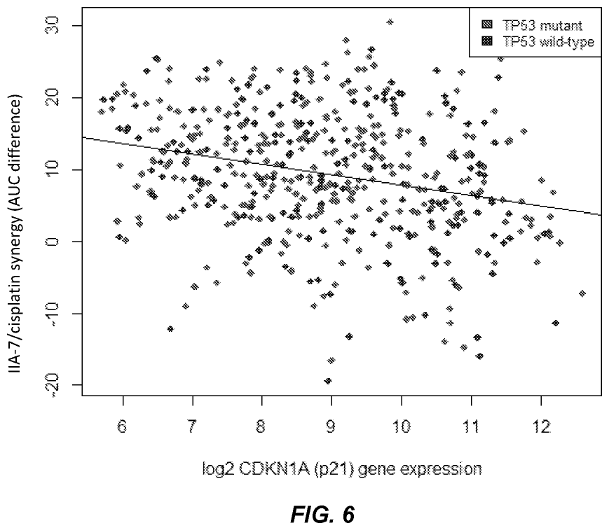

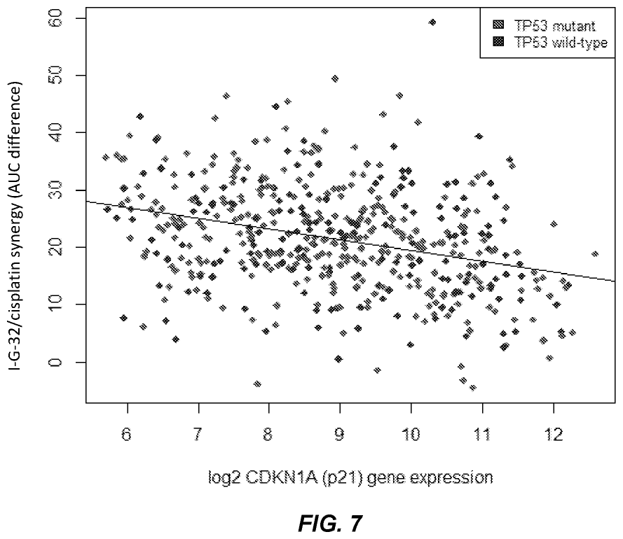

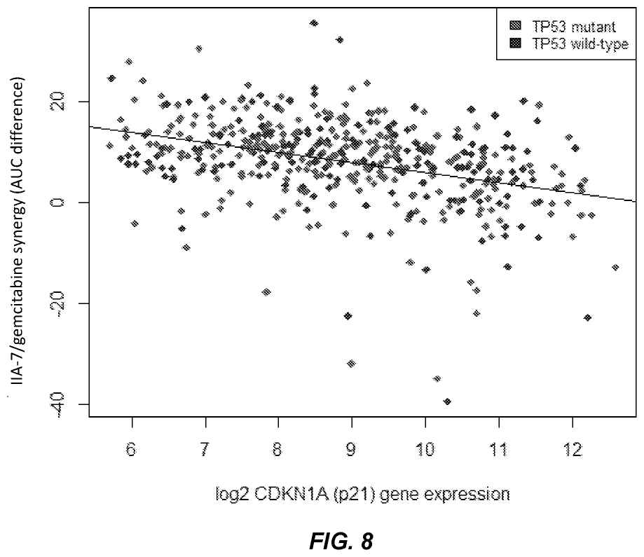

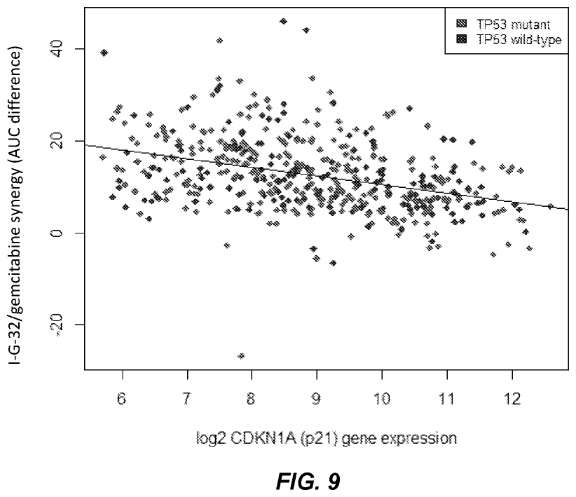

When assessed across 502 cancer cell lines (i.e., the set of cancer cell lines with gene expression data), the data showed a strong association between baseline CDKN1A gene expression and synergistic response across all combinations of ATR inhibitor with cisplatin or gemcitabine (ANOVA p value range: 8.4×10-14 to 8.7×10-6) (see FIGS. 6, 7, 8 and 9). Scatterplots illustrating this relationship between CDKN1A gene expression and response are shown in FIGS. 6, 7, 8 and 9.

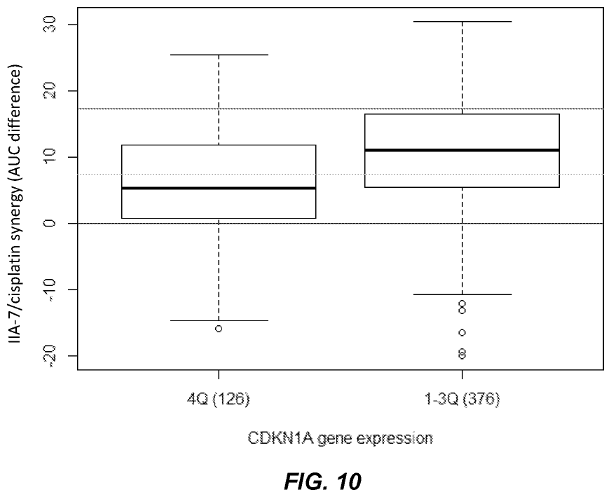

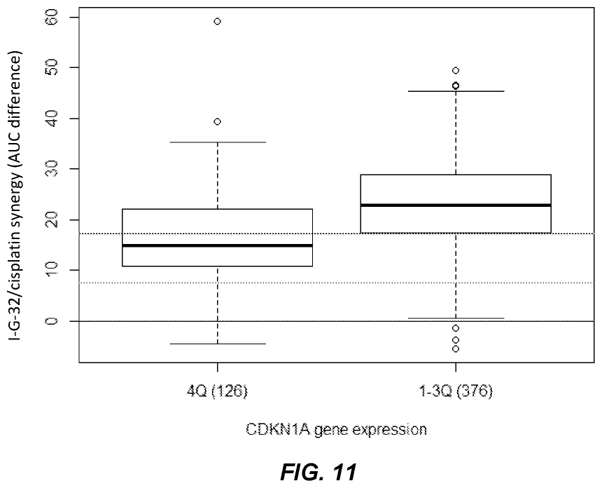

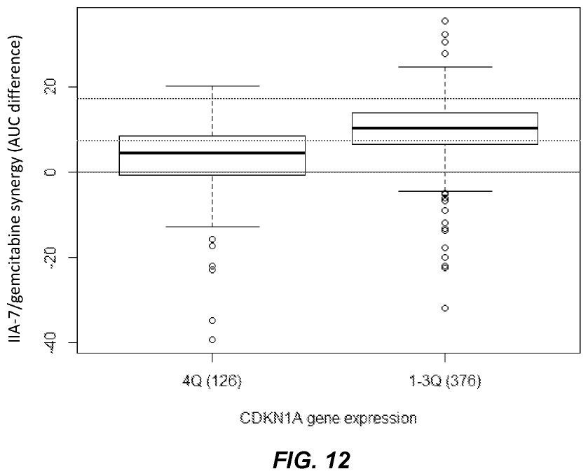

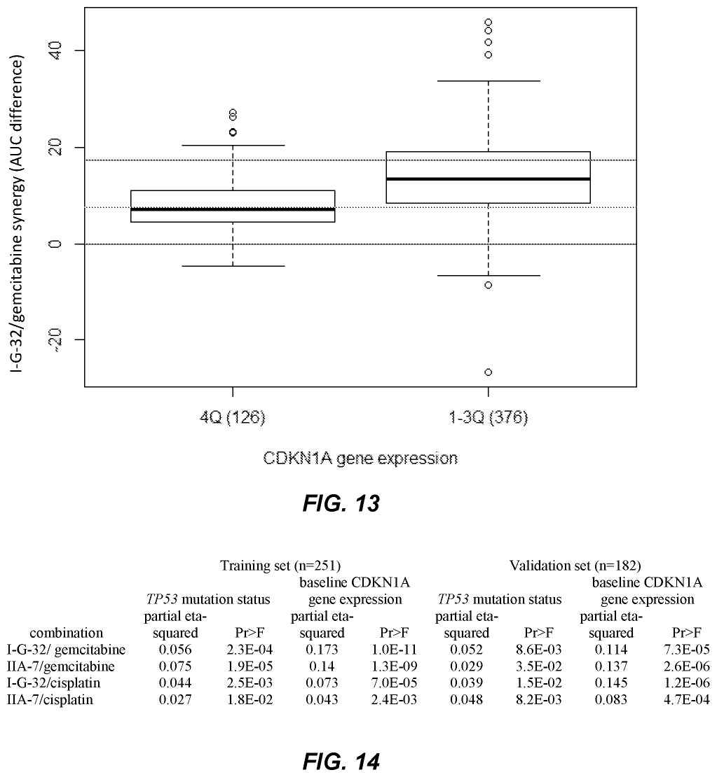

As an illustration of the potential use of baseline CDKN1A gene expression as a patient stratification biomarker, there is clear separation in ATR inhibitor synergy between the cell lines in the highest quartile of CDKN1A gene expression and the cell lines in the lowest three quartiles of CDKN1A gene expression (see FIGS. 10, 11, 12, and 13).

Conclusions. ATR inhibitor compound IIA-7 and compound I-G-32 are potent, selective inhibitors of ATR. The study presented herein demonstrate synergy of ATR inhibitors, compound IIA-7 and compound I-G-32, with the cytotoxic agents cisplatin and gemcitabine, and validated the association between TP53 mutational status and synergistic response to the combination of the ATR inhibitors with the genotoxic agents. A strong, statistically significant, relationship between TP53 mutation and response to all combination agents tested was observed in the panel of 552 cancer cell lines tested (FIGS. 2, 3, 4 and 5).

Further, the studies herein identified a functional marker of TP53, baseline CDKN1A gene expression, as a candidate predictive biomarker for synergistic response to combinations of ATR inhibitors with cisplatin and gemcitabine, a result which was validated in independent cell line subsets within this study. The role of CDKN1A as a downstream transcriptional target of TP53 serves as further confirmation of the role of the p53 pathway in the ATR inhibitor mechanism of action.

While various specific embodiments have been illustrated and described, it will be appreciated that various changes can be made without departing from the spirit and scope of the invention(s).

All publications, patents, patent applications and other documents cited in this application are hereby incorporated by reference in their entireties for all purposes to the same extent as if each individual publication, patent, patent application or other document were individually indicated to be incorporated by reference for all purposes.

1. A method of treating a patient having cancer, comprising administering to a patient with a cancer identified as having a reduced cyclin dependent kinase inhibitor 1A (CDKN1A) activity as compared to CDKN1A activity in control tissue or cell a therapeutically effective amount of an ATR inhibitor to sensitize the cancer to a DNA damaging agent.

2. The method of claim 1, further comprising administering to the patient a therapeutically effective amount of a DNA damaging agent.

3. The method of claim 1, wherein the cancer having a reduced CDKN1A activity is characterized by a synergistic growth inhibition response to the ATR inhibitor and the DNA damaging agent.

4. The method of claim 1, wherein the identifying is by:
measuring the level of cyclin dependent kinase inhibitor 1A (CDKN1A) activity in the cancer; and
comparing the measured CDKN1A activity to CDKN1A activity in a control tissue or cell.

5. The method of claim 1, further comprising detecting the presence or absence of an activity-attenuating or inactivating mutation in TP53 protein or a gene encoding the TP53 protein, wherein the cancer identified as having a reduced CDKN1A activity level compared to the CDKN1A activity in the control tissue or cell and the presence of an activity-attenuating or inactivating mutation in the TP53 protein or the gene encoding the TP53 protein is administered a therapeutically amount of the ATR inhibitor.

6. The method of claim 5, wherein the activity attenuating or inactivating mutation of TP53 is a loss of function mutation in the DNA binding domain, homo-oligomerization domain, or transactivation domain of TP53.

7-16. (canceled)

17. The method of claim 1, wherein the reduced CDKN1A activity is a CDKN1A activity level which is in the lower three quartiles of the CDKN1A activity in the control tissue or cell.

18. The method of claim 17, wherein the reduced CDKN1A activity is a CDKN1A activity level which is in the third or lower quartile of the CDKN1A activity in the control tissue or cell.

19. The method of claim 17, wherein the reduced CDKN1A activity is a CDKN1A activity level which is in the first quartile of the CDKN1A activity in the control tissue or cell.

20. The method of claim 1, wherein the reduced CDKN1A activity is a CDKN1A activity level which is about 75% or less, about 50% or less, or about 25% or less of the CDKN1A activity of the control tissue or cell.

21-37. (canceled)

38. The method of claim 1, wherein the CDKN1A activity is determined by (a) measuring CDKN1A protein expression, (b) measuring CDKN1A mRNA expression, (c) detecting the presence or absence of activity-attenuating or inactivating mutations in CDKN1A protein or a gene encoding the CDKN1A protein, or (d) combinations thereof.

39-47. (canceled)

48. The method of claim 38, wherein the CDKN1A activity is determined for a biological sample of the cancer obtained from the patient.

49. The method of claim 48, wherein the biological sample comprises a biopsy sample, lymphatic sample, or a blood sample containing the cancer.

50. The method of claim 1, wherein the ATR inhibitor is compound of Formula IA: 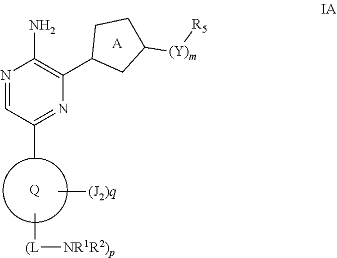
or a pharmaceutically acceptable salt thereof; wherein
Y is a C₁-C₁₀aliphatic chain wherein up to three methylene units of the aliphatic chain are optionally replaced with O, NR⁰, S, C(O) or S(O)₂;
Ring A is a 5 membered heteroaryl ring selected from 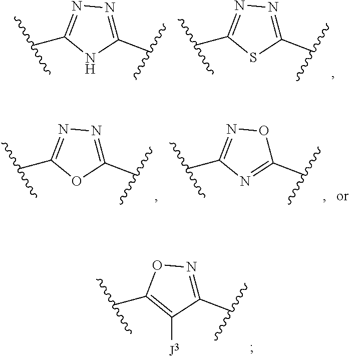
J³ is H or C₁-C₄alkyl, wherein 1 methylene unit of the alkyl group can optionally be replaced with O, NH, N(C₁-C₄alkyl), or S and optionally substituted with 1-3 halo;
Q is a 5-6 membered monocyclic aromatic ring containing 0-4 heteroatoms independently selected from nitrogen, oxygen, and sulfur; or an 8-10 membered bicyclic aromatic ring containing 0-6 heteroatoms independently selected from nitrogen, oxygen, and sulfur;
R⁵ is H; a 3-7 membered monocyclic fully saturated, partially unsaturated, or aromatic ring containing 0-4 heteroatoms independently selected from nitrogen, oxygen, and sulfur; an 8-10 membered bicyclic fully saturated, partially unsaturated, or aromatic ring containing 0-6 heteroatoms independently selected from nitrogen, oxygen, and sulfur; wherein R⁵ is optionally substituted with 1-5 J⁵ groups;
L is a C₁-C₄alkyl chain wherein up to two methylene units of the alkyl chain are optionally replaced with O, NR⁶, S, —C(O)—, —SO—, or —SO₂—;
R⁰ is H or C₁-C₆alkyl wherein one methylene unit of the alkyl chain can be optionally replaced with O, NH, N(C₁-C₄alkyl), or S;
R¹ is H or C₁-C₆alkyl;
R² is H, C₁-C₆alkyl, —(C₂-C₆alkyl)-Z or a 4-8 membered cyclic ring containing 0-2 nitrogen atoms; wherein said ring is bonded via a carbon atom and is optionally substituted with one occurrence of J^{Z};
or R¹ and R², taken together with the atom to which they are bound, form a 4-8 membered heterocyclic ring containing 1-2 heteroatoms selected from oxygen, nitrogen, and sulfur;
wherein said heterocyclic ring is optionally substituted with one occurrence of J^{Z1};
J^{Z1} is halo, CN, C₁-C₈aliphatic, —(X)ₜ—CN, or —(X)ₜ—Z, wherein said up to two methylene units of said C₁-C₈aliphatic can be optionally replaced with O, NR, S, P(O), C(O), S(O), or S(O)₂, wherein said C₁-C₈aliphatic is optionally substituted with halo, CN, or NO₂;
X is C₁-C₄alkyl;
each t, r and m is independently 0 or 1;
Z is —NR³R⁴;
R³ is H or C₁-C₂alkyl;
R⁴ is H or C₁-C₆alkyl;
or R³ and R⁴, taken together with the atom to which they are bound, form a 4-8 membered heterocyclic ring containing 1-2 heteroatoms selected from oxygen, nitrogen, and sulfur; wherein said ring is optionally substituted with one occurrence of J^{Z};
R⁶ is H, or C₁-C₆alkyl;
J^{Z} is independently NH₂, NH(C₁-C₄aliphatic), N(C₁-C₄aliphatic)₂, halogen, C₁-C₄aliphatic, OH, O(C₁-C₄aliphatic), NO₂, CN, CO₂H, CO(C₁-C₄aliphatic), CO₂(C₁-C₄aliphatic), O(haloC₁-C₄aliphatic), or haloC₁-C₄aliphatic;
J⁵ is halo, oxo, CN, NO₂, X¹—R, or —(X¹)ₚ-Q⁴;
X¹ is C₁-C₁₀aliphatic; wherein 1-3 methylene units of said C₁-C₁₀aliphatic are optionally replaced with —NR′—, —O—, —S—, C(═NR′), C(O), S(O)₂, or S(O), wherein X¹ is optionally and independently substituted with 1-4 occurrences of NH₂, NH(C₁-C₄aliphatic), N(C₁-C₄aliphatic)₂, halogen, C₁-C₄aliphatic, OH, O(C₁-C₄aliphatic), NO₂, CN, CO₂H, CO₂(C₁-C₄aliphatic), C(O)NH₂, C(O)NH(C₁-C₄aliphatic), C(O)N(C₁-C₄aliphatic)₂, SO(C₁-C₄aliphatic), SO₂(C₁-C₄aliphatic), SO₂NH(C₁-C₄aliphatic), NHC(O)(C₁-C₄aliphatic), N(C₁-C₄aliphatic)C(O)(C₁-C₄aliphatic), wherein said C₁-C₄aliphatic is optionally substituted with 1-3 occurrences of halo;
Q⁴ is a 3-8 membered saturated or unsaturated monocyclic ring having 0-4 heteroatoms independently selected from nitrogen, oxygen, and sulfur, or a 8-10 membered saturated or unsaturated bicyclic ring having 0-6 heteroatoms independently selected from nitrogen, oxygen, and sulfur; each Q⁴ is optionally substituted with 1-5 J^{Q4};
J^{Q4} is halo, CN, or C₁-C₄alkyl wherein up to 2 methylene units are optionally replaced with 0, NR*, S, C(O), S(O), or S(O)₂;
R is H or C₁-C₄alkyl wherein said C₁-C₄alkyl is optionally substituted with 1-4 halo;
J² is halo; CN; a 5-6 membered aromatic or nonaromatic monocyclic ring having 0-3 heteroatoms selected from oxygen, nitrogen, and sulfur; or a C₁-C₁₀aliphatic group wherein up to 2 methylene units are optionally replaced with O, NR″, C(O), S, S(O), or S(O)₂; wherein said C₁-C₁₀aliphatic group is optionally substituted with 1-3 halo or CN; and said monocyclic ring is optionally substituted with 1-3 occurrences of halo; CN; a C₃-C₆cycloalkyl; a 3-7 membered heterocyclyl containing 0-2 heteroatoms selected from oxygen, nitrogen, and sulfur; or a C₁-C₄alkyl wherein up to one methylene unit of the alkyl chain is optionally replaced with O, NR″, or S; and wherein said C₁-C₄alkyl is optionally substituted with 1-3 halo;
q is 0, 1, or 2;
p is 0 or 1;
R′, R″, and R* are each independently H, C₁-C₄alkyl, or is absent; wherein said C₁-C₄alkyl is optionally substituted with 1-4 halo.

51. The method of claim 50, wherein the ATR inhibitor is compound of the following structure (IIA-7): 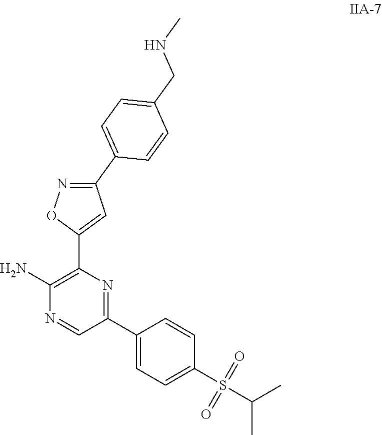 [SMILES: CNCc1ccc(-c2cc(-c3nc(-c4ccc(S(=O)(=O)C(C)C)cc4)cnc3N)on2)cc1]
or a pharmaceutically acceptable salt thereof.

52. The method of claim 1, wherein the ATR inhibitor is compound of the ATR inhibitor is a compound of Formula I: 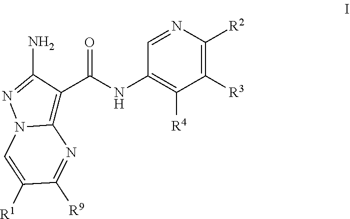
or a pharmaceutically acceptable salt thereof, wherein:
R¹ is independently selected from —C(J¹)₂CN, halo, -(L)ₖ-W, and M;
R⁹ is independently selected from H, —C(J¹)₂CN, halo, -(L)ₖ-W, and M;
J¹ is independently selected from H and C₁-C₂alkyl; or
two occurrences of J¹, together with the carbon atom to which they are attached, form a 3-4 membered optionally substituted carbocyclic ring;
k is 0 or 1;
M and L are a C₁-C₈aliphatic, wherein up to three methylene units are optionally replaced with —O—, —NR—, —C(O)—, or —S(O)ₙ—, each M and L¹ is optionally substituted with 0-3 occurrences of J^{LM};
J^{LM} is independently selected from halo, —CN, and a C₁-C₄aliphatic chain wherein up to two methylene units of the aliphatic chain are optionally replaced with —O—, —NR—, —C(O)—, or —S(O)ₙ—;
W is independently selected from a 3-7 membered fully saturated, partially unsaturated, or aromatic monocyclic ring having 0-3 heteroatoms selected from oxygen, nitrogen and sulfur; and a 7-12 membered fully saturated, partially unsaturated, or aromatic bicyclic ring having 0-5 heteroatoms selected from oxygen, nitrogen, and sulfur; wherein W is optionally substituted with 0-5 occurrences of J^{W};
J^{W} is independently selected from —CN, halo, —CF₃; a C₁-C₄aliphatic wherein up to two methylene units are optionally replaced with —O—, —NR—, —C(O)—, or —S(O)ₙ—; and a 3-6 membered non-aromatic ring having 0-2 heteroatoms selected from oxygen, nitrogen, and sulfur; or two occurrences of J^{W} on the same atom, together with atom to which they are joined, form a 3-6 membered ring having 0-2 heteroatoms selected from oxygen, nitrogen, and sulfur; or two occurrences of J^{W}, together with W, form a 6-10 membered saturated or partially unsaturated bridged ring system;
R² is independently selected from H; halo; —CN; NH₂; a C₁-C₂alkyl optionally substituted with 0-3 occurrences of fluoro; and a C₁-C₃aliphatic chain wherein up to two methylene units of the aliphatic chain are optionally replaced with —O—, —NR—, —C(O)—, or —S(O)ₙ;
R³ is independently selected from H; halo; C₁-C₄alkyl optionally substituted with 1-3 occurrences of halo; C₃-C₄cycloalkyl; 3-4 membered heterocyclyl; —CN; and a C₁-C₃aliphatic chain wherein up to two methylene units of the aliphatic chain are optionally replaced with —O—, —NR—, —C(O)—, or —S(O)ₙ;
R⁴ is independently selected from Q¹ and a C₁-C₁₀aliphatic chain wherein up to four methylene units of the aliphatic chain are optionally replaced with —O—, —NR—, —C(O)—, or —S(O)ₙ—; each R⁴ is optionally substituted with 0-5 occurrences of J^{Q}; or
R³ and R⁴, taken together with the atoms to which they are bound, form a 5-6 membered aromatic or non-aromatic ring having 0-2 heteroatoms selected from oxygen, nitrogen and sulfur; the ring formed by R³ and R⁴ is optionally substituted with 0-3 occurrences of J^{Z};
Q¹ is independently selected from a 3-7 membered fully saturated, partially unsaturated, or aromatic monocyclic ring, the 3-7 membered ring having 0-3 heteroatoms selected from oxygen, nitrogen and sulfur; and an 7-12 membered fully saturated, partially unsaturated, or aromatic bicyclic ring having 0-5 heteroatoms selected from oxygen, nitrogen, and sulfur;
J^{z} is independently selected from C₁-C₆aliphatic, ═O, halo, and →O;
J^{Q} is independently selected from —CN; halo; ═O; Q²; and a C₁-C₈aliphatic chain wherein up to three methylene units of the aliphatic chain are optionally replaced with —O—, —NR—, —C(O)—, or —S(O)ₙ—; each occurrence of J^{Q} is optionally substituted by 0-3 occurrences of J^{R}; or two occurrences of J^{Q} on the same atom, taken together with the atom to which they are joined, form a 3-6 membered ring having 0-2 heteroatoms selected from oxygen, nitrogen, and sulfur; wherein the ring formed by two occurrences of J^{Q} is optionally substituted with 0-3 occurrences of J^{X}; or two occurrences of J^{Q}, together with Q¹, form a 6-10 membered saturated or partially unsaturated bridged ring system;
Q² is independently selected from a 3-7 membered fully saturated, partially unsaturated, or aromatic monocyclic ring having 0-3 heteroatoms selected from oxygen, nitrogen, and sulfur; and an 7-12 membered fully saturated, partially unsaturated, or aromatic bicyclic ring having 0-5 heteroatoms selected from oxygen, nitrogen, and sulfur;
J^{R} is independently selected from —CN; halo; ═O; →O, Q³; and a C₁-C₆aliphatic chain wherein up to three methylene units of the aliphatic chain are optionally replaced with —O—, —NR—, —C(O)—, or —S(O)ₙ—; each J^{R} is optionally substituted with 0-3 occurrences of J^{T}; or two occurrences of J^{R} on the same atom, together with the atom to which they are joined, form a 3-6 membered ring having 0-2 heteroatoms selected from oxygen, nitrogen, and sulfur; wherein the ring formed by two occurrences of J^{R} is optionally substituted with 0-3 occurrences of J^{X}; or two occurrences of J^{R}, together with Q², form a 6-10 membered saturated or partially unsaturated bridged ring system;
Q³ is a 3-7 membered fully saturated, partially unsaturated, or aromatic monocyclic ring having 0-3 heteroatoms selected from oxygen, nitrogen, or sulfur; or an 7-12 membered fully saturated, partially unsaturated, or aromatic bicyclic ring having 0-5 heteroatoms selected from oxygen, nitrogen, and sulfur;
J^{X} is independently selected from-CN; ═O; halo; and a C₁-C₄aliphatic chain, wherein up to two methylene units of the aliphatic chain are optionally replaced with —O—, —NR—, —C(O)—, or —S(O)ₙ—;
J^{T} is independently selected from halo, —CN; →O; ═O; —OH; a C₁-C₆aliphatic chain wherein up to two methylene units of the aliphatic chain are optionally replaced with —O—, —NR—, —C(O)—, or —S(O)ₙ—; and a 3-6 membered non-aromatic ring having 0-2 heteroatoms selected from oxygen, nitrogen, and sulfur; each occurrence of J^{T} is optionally substituted with 0-3 occurrences of J^{M}; or two occurrences of J^{T} on the same atom, together with the atom to which they are joined, form a 3-6 membered ring having 0-2 heteroatoms selected from oxygen, nitrogen, and sulfur; or two occurrences of J^{T}, together with Q³, form a 6-10 membered saturated or partially unsaturated bridged ring system;
J^{M} is independently selected from halo and C₁-C₆aliphatic;
n is 0, 1 or 2; and
R is independently selected from H and C₁-C₄aliphatic.

53. The method of claim 1, wherein the DNA damaging agent when present comprises ionizing radiation, platinating agent, topoisomerase I (Topo I) inhibitor, topoisomerase II (Topo II) inhibitor, anti-metabolite, alkylating agent, anti-cancer antibiotic, or combinations thereof.

54. The method of claim 53, wherein the DNA damaging agent comprises a platinating agent.

55. The method of claim 54, wherein the platinating agent comprises cisplatin, oxaliplatin, or carboplatin.

56. The method of claim 53, wherein the DNA damaging agent comprises an antimetabolite.

57. The method of claim 56, wherein the anti-metabolite comprises cytarabine, gemcitabine, capecitabine, or 5-fluorouracil (5-FU).

58. The method of claim 1, wherein the DNA damaging agent, when present, comprises a DNA damage enhancing agent.

59. The method of claim 58, wherein the DNA damage enhancing agent is a PARP inhibitor.

60. The method of claim 59, wherein the DNA damage enhancing agent is a Chk1 inhibitor.

61. The method of claim 1, wherein the cancer is lung cancer, ovarian cancer, endometrial cancer, pancreatic cancer, head and neck cancer, esophageal cancer, breast cancer and colorectal cancer.

62. The method of claim 1, wherein the cancer is a hematologic cancer.

63. The method of claim 62, wherein the hematological cancer is a lymphoma or a leukemia.

64. (canceled)

65. A method of treating a patient having a cancer, comprising:
measuring the level of cyclin dependent kinase inhibitor 1A (CDKN1A) activity in a cancer of a patient;
comparing the measured CDKN1A activity to CDKN1A activity in a control tissue or cell; and
(a) treating the patient with a cancer treatment regimen which does not include treatment with an ATR inhibitor in combination with a DNA damaging agent if the cancer is identified as having a CDKN1A activity which is substantially similar to CDKN1A activity in control tissue or cell; and
(b) treating the patient with a cancer treatment regimen which includes treatment with an ATR inhibitor in combination with a DNA damaging agent if the cancer is identified as having a CDKN1A activity which is reduced as compared to CDKN1A activity in control tissue or cell.

66. (canceled)


SEQUENCE LISTING (2 sequences)

SEQ ID NO: 1 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 2 - DNA, 495 bp, Homo sapiens
atgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaa